(19) United States
(12) Patent Application Publication
(10) Pub. No.: US 2024/0182892 A1
(43) Pub. Date: Jun. 6, 2024

(54) DOUBLE-STRANDED SIRNA HAVING PATTERNED CHEMICAL MODIFICATIONS

(71) Applicant: Atalanta Therapeutics, Inc., Boston, MA (US)

(72) Inventors: Matthew HASSLER, Boston, MA (US); Daniel CURTIS, Belmont, MA (US); Bruno Miguel DA CRUZ GODINHO, Reading, MA (US)

(21) Appl. No.: 18/283,666

(22) PCT Filed: Mar. 24, 2022
(86) PCT No.: PCT/US2022/021790

§ 371 Date: Sep. 22, 2023

Related U.S. Application Data
(60) Provisional application No. 63/165,543, filed on Mar. 24, 2021.

(51) Int. Cl. C12N 15/113; A61K 31/713

(52) U.S. Cl. CPC C12N 15/113; A61K 31/713; C12N 2310/14; C12N 2310/31; C12N 2310/315; C12N 2310/321; C12N 2310/322; C12N 2310/346; C12N 2310/3515

(57) ABSTRACT

The present disclosure provides single- or double-stranded short interfering RNA (siRNA) molecules having specific patterns of nucleoside modifications and internucleoside linkage modifications, as pharmaceutical compositions including the same. The siRNA molecules may be branched siRNA molecules, such as di-branched, tri-branched, ortetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a disease.

### TECHNICAL FIELD

This disclosure relates to novel short interfering RNA (siRNA) molecules useful for RNA silencing by way of, e.g., RNA interference (RNAi), containing patterns of chemically-modified ribonucleotides, patterns of chemically-modified internucleoside linkages, branched structures, hydrophobic moieties, and/or 5′ phosphorus stabilizing moieties.

### BACKGROUND

In many species, introduction of double-stranded RNA (dsRNA) induces potent and specific gene silencing by way of RNA interference (RNAi). This phenomenon occurs in both plants and animals and has roles in viral defense and transposon silencing mechanisms. Short interfering RNAs (siRNAs), which are generally much shorter than the target gene, have been shown to be effective at gene silencing and are, therefore, useful as therapeutic agents for silencing genes to restore genetic and biochemical pathway activity from a disease state towards a normal, healthy state.

siRNAs containing chemically-modified ribonucleosides and/or chemically-modified linkers are known to exhibit increased nuclease resistance relative to the corresponding unmodified siRNAs, while maintaining RNAi activity. There remains a need for siRNA molecules having improved nuclease resistance and gene silencing efficacy.

### SUMMARY OF THE DISCLOSURE

In a first aspect, the present disclosure provides a small interfering RNA (siRNA) molecule including an antisense strand and a sense strand having complementarity to the antisense strand, wherein the antisense strand includes a structure represented by Formula I, wherein Formula I is, in the 5′-to-3′ direction:

A-B-(A′)ⱼ-C-P²-D-P¹-(C′-P¹)ₖ-C′ Formula I;

wherein A is represented by the formula C-P¹-D-P¹; each A′, independently, is represented by the formula C-P²-D-P²; B is represented by the formula C-P²-D-P²-D-P²-D-P²; each C, independently, is a 2′-O-methyl (2′-O-Me) ribonucleoside; each C′, independently, is a 2′-O-Me ribonucleoside or a 2′-fluoro (2′-F) ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7; and k is an integer from 1 to 7.

In some embodiments, the antisense strand includes a structure represented by formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand includes a structure represented by Formula II, wherein Formula II is, in the 5′-to-3′ direction:

A-B-(A′)ⱼ-C-P²-D-P¹-(C-P¹)ₖ-C′ Formula II;

wherein A is represented by the formula C-P¹-D-P¹; each A′, independently, is represented by the formula C-P²-D-P²; B is represented by the formula C-P²-D-P²-D-P²-D-P²; each C, independently, is a 2′-O -methyl (2′-O-Me) ribonucleoside; each C′, independently, is a 2′-O-Me ribonucleoside or a 2′-fluoro (2′-F) ribonucleoside, each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7).

In some embodiments, the antisense strand includes a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand includes a structure represented by Formula III, wherein Formula III is, in the 5′-to-3′ direction:

E-(A′)ₘ-F Formula III;

wherein E is represented by the formula (C-P¹)₂; F is represented by the formula (C-P²)₃-D-P¹-C-P¹-C, (C-P²)₃-D-P²-C-P²-C, or (C-P²)₃-D-P²-C-P²-D; A′, C, D, P¹, and P² are as defined in Formula I; and m is an integer from 1 to 7 (e.g., 1, 2, 3, 4, 5, 6, or 7).

In some embodiments, j is 4 and k is 4. In some embodiments, m is 4.

In some embodiments, the sense strand includes a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand includes a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand includes a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand includes a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, O represents a phosphodiester internucleoside linkage, and S represents a phosphorothioate internucleoside linkage.

In another aspect, the present disclosure provides an siRNA molecule including an antisense strand and a sense strand having complementarity to the antisense strand, wherein the antisense strand includes a structure represented by Formula IV, wherein Formula IV is, in the 5′-to-3′ direction:

A-(A′)ⱼ-C-P²-B-(C-P¹)ₖ-C′ Formula IV;

wherein A is represented by the formula C-P¹-D-P¹; each A′, independently, is represented by the formula C-P²-D-P²; B is represented by the formula D-P′-C-P¹-D-P¹; each C, independently, is a 2′-O-Me ribonucleoside; each C′, independently, is a 2′-O-Me ribonucleoside or a 2′-F ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, of the foregoing aspect, the antisense strand includes a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the foregoing aspect, the sense strand includes a structure represented by Formula V, wherein Formula V is, in the 5′-to-3′ direction:

E-(A′)ₘ-C-P²-F Formula V;

wherein E is represented by the formula (C-P¹)₂; F is represented by the formula D-P¹-C-P¹-C, D-P²-C-P²-C, D-P′-C-P′-D, or D-P²-C-P²-D; A′, C, D, P¹, and P² are as defined in Formula IV; and m is an integer from 1 to 7 (e.g., 1, 2, 3, 4, 5, 6, or 7).

In some embodiments of the foregoing aspect, j is 6 and k is 2. In some embodiments, m is 5.

In some embodiments of the foregoing aspect, the sense strand includes a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the foregoing aspect, the sense strand includes a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′-O-Me ribonucleoside, B represents a 2′-F ribonucleoside, O represents a phosphodiester internucleoside linkage, and S represents a phosphorothioate internucleoside linkage. In some embodiments of the foregoing aspect, the sense strand includes a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the foregoing aspect, the sense strand includes a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, O represents a phosphodiester internucleoside linkage, and S represents a phosphorothioate internucleoside linkage.

In another aspect, the present disclosure provides an siRNA molecule including an antisense strand and a sense strand having complementarity to the antisense strand, wherein the antisense strand includes a structure represented by Formula VI, wherein Formula VI is, in the 5′-to-3′ direction:

A-Bⱼ-E-Bₖ-E-F-Gₗ-D-P¹-C′ Formula VI;

wherein A is represented by the formula C-P¹-D-P¹; each B, independently, is represented by the formula C-P²; each C, independently, is a 2′-O-Me ribonucleoside; each C′, independently, is a 2′-O-Me ribonucleoside or a 2′-F ribonucleoside; each D, independently, is a 2′-F ribonucleoside; each E, independently, is represented by the formula D-P²-C-P²; F is represented by the formula D-P¹-C-P¹; each G, independently, is represented by the formula C-P¹; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7).

In some embodiments of the foregoing aspect, the antisense strand includes a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, O represents a phosphodiester internucleoside linkage, and S represents a phosphorothioate internucleoside linkage. In some embodiments of the foregoing aspect, the sense strand includes a structure represented by Formula VII, wherein Formula VII is, in the 5′-to-3′ direction:

H-Bₘ-Iₙ-A′-Bₒ-H-C Formula VII;

wherein A′ is represented by the formula C-P²-D-P²; each H, independently, is represented by the formula (C-P¹)₂; each I, independently, is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7).

In some embodiments of the foregoing aspect, j is 3, k is 6, and l is 2. In some embodiments, m is 3, n is 3, and o is 3.

In some embodiments of the foregoing aspect, the sense strand includes a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of any of the foregoing aspects, the antisense strand further includes a 5′ phosphorus stabilizing moiety at the 5′ end of the antisense strand. In some embodiments of any of the foregoing aspects, the sense strand further includes a 5′ phosphorus stabilizing moiety at the 5′ end of the antisense strand. In some embodiments, the 5′ phosphorus stabilizing moiety is represented by any one of Formulas VIII-XV:

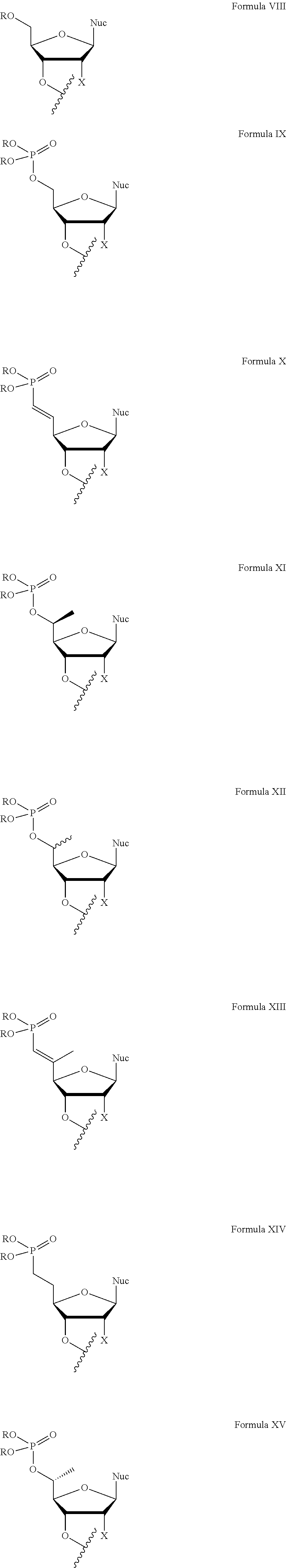

wherein Nuc represents a nucleobase and R represents an optionally substituted alkyl, optionally substituted alkenyl, optionally substituted alkynyl (e.g., an optionally substituted C1-C6 alkyl, optionally substituted C2-C6 alkenyl, or optionally substituted C2-C6 alkynyl), phenyl, benzyl, hydroxy, or hydrogen. In some embodiments, the nucleobase is an adenine, uracil, guanine, thymine, or cytosine. In some embodiments, the 5′ phosphorus stabilizing moiety is (E)-vinylphosphonate represented by Formula X.

In some embodiments of any one of the foregoing aspects, the siRNA molecule further includes a hydrophobic moiety at the 5′ or the 3′ end of the siRNA molecule. In some embodiments, the hydrophobic moiety is selected from a group consisting of cholesterol, vitamin D, and tocopherol.

In some embodiments of any one of the foregoing aspects, the length of the antisense strand is 10 to 30 (e.g., 12 to 28, 14 to 26, 16 to 24, or 18 to 22) nucleotides. In some embodiments, the length of the antisense strand is 15 to 25 (e.g., 16 to 24, 17 to 23, 18 to 22, or 19 to 21) nucleotides. In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments of any one of the foregoing aspects, the length of the sense strand is between 12 and 20 (e.g., between 13 and 19, between 14 and 18, or between 15 and 17) nucleotides. In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, the antisense strand is 18 nucleotides in length and the sense strand is 14 nucleotides in length.

In some embodiments, the antisense strand is 18 nucleotides in length and the sense strand is 15 nucleotides in length.

In some embodiments, the antisense strand is 18 nucleotides in length and the sense strand is 16 nucleotides in length.

In some embodiments, the antisense strand is 18 nucleotides in length and the sense strand is 17 nucleotides in length.

In some embodiments, the antisense strand is 18 nucleotides in length and the sense strand is 18 nucleotides in length.

In some embodiments, the antisense strand is 19 nucleotides in length and the sense strand is 14 nucleotides in length.

In some embodiments, the antisense strand is 19 nucleotides in length and the sense strand is 15 nucleotides in length.

In some embodiments, the antisense strand is 19 nucleotides in length and the sense strand is 16 nucleotides in length.

In some embodiments, the antisense strand is 19 nucleotides in length and the sense strand is 17 nucleotides in length.

In some embodiments, the antisense strand is 19 nucleotides in length and the sense strand is 18 nucleotides in length.

In some embodiments, the antisense strand is 19 nucleotides in length and the sense strand is 19 nucleotides in length.

In some embodiments, the antisense strand is 20 nucleotides in length and the sense strand is 14 nucleotides in length.

In some embodiments, the antisense strand is 20 nucleotides in length and the sense strand is 15 nucleotides in length.

In some embodiments, the antisense strand is 20 nucleotides in length and the sense strand is 16 nucleotides in length.

In some embodiments, the antisense strand is 20 nucleotides in length and the sense strand is 17 nucleotides in length.

In some embodiments, the antisense strand is 20 nucleotides in length and the sense strand is 18 nucleotides in length.

In some embodiments, the antisense strand is 20 nucleotides in length and the sense strand is 19 nucleotides in length.

In some embodiments, the antisense strand is 20 nucleotides in length and the sense strand is 20 nucleotides in length.

In some embodiments, the antisense strand is 21 nucleotides in length and the sense strand is 14 nucleotides in length.

In some embodiments, the antisense strand is 21 nucleotides in length and the sense strand is 15 nucleotides in length.

In some embodiments, the antisense strand is 21 nucleotides in length and the sense strand is 16 nucleotides in length.

In some embodiments, the antisense strand is 21 nucleotides in length and the sense strand is 17 nucleotides in length.

In some embodiments, the antisense strand is 21 nucleotides in length and the sense strand is 18 nucleotides in length.

In some embodiments, the antisense strand is 21 nucleotides in length and the sense strand is 19 nucleotides in length.

In some embodiments, the antisense strand is 21 nucleotides in length and the sense strand is 20 nucleotides in length.

In some embodiments, the antisense strand is 21 nucleotides in length and the sense strand is 21 nucleotides in length.

In some embodiments, the antisense strand is 22 nucleotides in length and the sense strand is 14 nucleotides in length.

In some embodiments, the antisense strand is 22 nucleotides in length and the sense strand is 15 nucleotides in length.

In some embodiments, the antisense strand is 22 nucleotides in length and the sense strand is 16 nucleotides in length.

In some embodiments, the antisense strand is 22 nucleotides in length and the sense strand is 17 nucleotides in length.

In some embodiments, the antisense strand is 22 nucleotides in length and the sense strand is 18 nucleotides in length.

In some embodiments, the antisense strand is 22 nucleotides in length and the sense strand is 19 nucleotides in length.

In some embodiments, the antisense strand is 22 nucleotides in length and the sense strand is 20 nucleotides in length.

In some embodiments, the antisense strand is 22 nucleotides in length and the sense strand is 21 nucleotides in length.

In some embodiments, the antisense strand is 22 nucleotides in length and the sense strand is 22 nucleotides in length.

In some embodiments, the antisense strand is 23 nucleotides in length and the sense strand is 14 nucleotides in length.

In some embodiments, the antisense strand is 23 nucleotides in length and the sense strand is 15 nucleotides in length.

In some embodiments, the antisense strand is 23 nucleotides in length and the sense strand is 16 nucleotides in length.

In some embodiments, the antisense strand is 23 nucleotides in length and the sense strand is 17 nucleotides in length.

In some embodiments, the antisense strand is 23 nucleotides in length and the sense strand is 18 nucleotides in length.

In some embodiments, the antisense strand is 23 nucleotides in length and the sense strand is 19 nucleotides in length.

In some embodiments, the antisense strand is 23 nucleotides in length and the sense strand is 20 nucleotides in length.

In some embodiments, the antisense strand is 23 nucleotides in length and the sense strand is 21 nucleotides in length.

In some embodiments, the antisense strand is 23 nucleotides in length and the sense strand is 22 nucleotides in length.

In some embodiments, the antisense strand is 23 nucleotides in length and the sense strand is 23 nucleotides in length.

In some embodiments, the antisense strand is 24 nucleotides in length and the sense strand is 14 nucleotides in length.

In some embodiments, the antisense strand is 24 nucleotides in length and the sense strand is 15 nucleotides in length.

In some embodiments, the antisense strand is 24 nucleotides in length and the sense strand is 16 nucleotides in length.

In some embodiments, the antisense strand is 24 nucleotides in length and the sense strand is 17 nucleotides in length.

In some embodiments, the antisense strand is 24 nucleotides in length and the sense strand is 18 nucleotides in length.

In some embodiments, the antisense strand is 24 nucleotides in length and the sense strand is 19 nucleotides in length.

In some embodiments, the antisense strand is 24 nucleotides in length and the sense strand is 20 nucleotides in length.

In some embodiments, the antisense strand is 24 nucleotides in length and the sense strand is 21 nucleotides in length.

In some embodiments, the antisense strand is 24 nucleotides in length and the sense strand is 22 nucleotides in length.

In some embodiments, the antisense strand is 24 nucleotides in length and the sense strand is 23 nucleotides in length.

In some embodiments, the antisense strand is 24 nucleotides in length and the sense strand is 24 nucleotides in length.

In some embodiments, the antisense strand is 25 nucleotides in length and the sense strand is 14 nucleotides in length.

In some embodiments, the antisense strand is 25 nucleotides in length and the sense strand is 15 nucleotides in length.

In some embodiments, the antisense strand is 25 nucleotides in length and the sense strand is 16 nucleotides in length.

In some embodiments, the antisense strand is 25 nucleotides in length and the sense strand is 17 nucleotides in length.

In some embodiments, the antisense strand is 25 nucleotides in length and the sense strand is 18 nucleotides in length.

In some embodiments, the antisense strand is 25 nucleotides in length and the sense strand is 19 nucleotides in length.

In some embodiments, the antisense strand is 25 nucleotides in length and the sense strand is 20 nucleotides in length.

In some embodiments, the antisense strand is 25 nucleotides in length and the sense strand is 21 nucleotides in length.

In some embodiments, the antisense strand is 25 nucleotides in length and the sense strand is 22 nucleotides in length.

In some embodiments, the antisense strand is 25 nucleotides in length and the sense strand is 23 nucleotides in length.

In some embodiments, the antisense strand is 25 nucleotides in length and the sense strand is 24 nucleotides in length.

In some embodiments, the antisense strand is 25 nucleotides in length and the sense strand is 25 nucleotides in length.

In some embodiments, the antisense strand is 26 nucleotides in length and the sense strand is 14 nucleotides in length.

In some embodiments, the antisense strand is 26 nucleotides in length and the sense strand is 15 nucleotides in length.

In some embodiments, the antisense strand is 26 nucleotides in length and the sense strand is 16 nucleotides in length.

In some embodiments, the antisense strand is 26 nucleotides in length and the sense strand is 17 nucleotides in length.

In some embodiments, the antisense strand is 26 nucleotides in length and the sense strand is 18 nucleotides in length.

In some embodiments, the antisense strand is 26 nucleotides in length and the sense strand is 19 nucleotides in length.

In some embodiments, the antisense strand is 26 nucleotides in length and the sense strand is 20 nucleotides in length.

In some embodiments, the antisense strand is 26 nucleotides in length and the sense strand is 21 nucleotides in length.

In some embodiments, the antisense strand is 26 nucleotides in length and the sense strand is 22 nucleotides in length.

In some embodiments, the antisense strand is 26 nucleotides in length and the sense strand is 23 nucleotides in length.

In some embodiments, the antisense strand is 26 nucleotides in length and the sense strand is 24 nucleotides in length.

In some embodiments, the antisense strand is 26 nucleotides in length and the sense strand is 25 nucleotides in length.

In some embodiments, the antisense strand is 26 nucleotides in length and the sense strand is 26 nucleotides in length.

In some embodiments, the antisense strand is 27 nucleotides in length and the sense strand is 14 nucleotides in length.

In some embodiments, the antisense strand is 27 nucleotides in length and the sense strand is 15 nucleotides in length.

In some embodiments, the antisense strand is 27 nucleotides in length and the sense strand is 16 nucleotides in length.

In some embodiments, the antisense strand is 27 nucleotides in length and the sense strand is 17 nucleotides in length.

In some embodiments, the antisense strand is 27 nucleotides in length and the sense strand is 18 nucleotides in length.

In some embodiments, the antisense strand is 27 nucleotides in length and the sense strand is 19 nucleotides in length.

In some embodiments, the antisense strand is 27 nucleotides in length and the sense strand is 20 nucleotides in length.

In some embodiments, the antisense strand is 27 nucleotides in length and the sense strand is 21 nucleotides in length.

In some embodiments, the antisense strand is 27 nucleotides in length and the sense strand is 22 nucleotides in length.

In some embodiments, the antisense strand is 27 nucleotides in length and the sense strand is 23 nucleotides in length.

In some embodiments, the antisense strand is 27 nucleotides in length and the sense strand is 24 nucleotides in length.

In some embodiments, the antisense strand is 27 nucleotides in length and the sense strand is 25 nucleotides in length.

In some embodiments, the antisense strand is 27 nucleotides in length and the sense strand is 26 nucleotides in length.

In some embodiments, the antisense strand is 27 nucleotides in length and the sense strand is 27 nucleotides in length.

In some embodiments, the antisense strand is 28 nucleotides in length and the sense strand is 14 nucleotides in length.

In some embodiments, the antisense strand is 28 nucleotides in length and the sense strand is 15 nucleotides in length.

In some embodiments, the antisense strand is 28 nucleotides in length and the sense strand is 16 nucleotides in length.

In some embodiments, the antisense strand is 28 nucleotides in length and the sense strand is 17 nucleotides in length.

In some embodiments, the antisense strand is 28 nucleotides in length and the sense strand is 18 nucleotides in length.

In some embodiments, the antisense strand is 28 nucleotides in length and the sense strand is 19 nucleotides in length.

In some embodiments, the antisense strand is 28 nucleotides in length and the sense strand is 20 nucleotides in length.

In some embodiments, the antisense strand is 28 nucleotides in length and the sense strand is 21 nucleotides in length.

In some embodiments, the antisense strand is 28 nucleotides in length and the sense strand is 22 nucleotides in length.

In some embodiments, the antisense strand is 28 nucleotides in length and the sense strand is 23 nucleotides in length.

In some embodiments, the antisense strand is 28 nucleotides in length and the sense strand is 24 nucleotides in length.

In some embodiments, the antisense strand is 28 nucleotides in length and the sense strand is 25 nucleotides in length.

In some embodiments, the antisense strand is 28 nucleotides in length and the sense strand is 26 nucleotides in length.

In some embodiments, the antisense strand is 28 nucleotides in length and the sense strand is 27 nucleotides in length.

In some embodiments, the antisense strand is 28 nucleotides in length and the sense strand is 28 nucleotides in length.

In some embodiments, the antisense strand is 29 nucleotides in length and the sense strand is 14 nucleotides in length.

In some embodiments, the antisense strand is 29 nucleotides in length and the sense strand is 15 nucleotides in length.

In some embodiments, the antisense strand is 29 nucleotides in length and the sense strand is 16 nucleotides in length.

In some embodiments, the antisense strand is 29 nucleotides in length and the sense strand is 17 nucleotides in length.

In some embodiments, the antisense strand is 29 nucleotides in length and the sense strand is 18 nucleotides in length.

In some embodiments, the antisense strand is 29 nucleotides in length and the sense strand is 19 nucleotides in length.

In some embodiments, the antisense strand is 29 nucleotides in length and the sense strand is 20 nucleotides in length.

In some embodiments, the antisense strand is 29 nucleotides in length and the sense strand is 21 nucleotides in length.

In some embodiments, the antisense strand is 29 nucleotides in length and the sense strand is 22 nucleotides in length.

In some embodiments, the antisense strand is 29 nucleotides in length and the sense strand is 23 nucleotides in length.

In some embodiments, the antisense strand is 29 nucleotides in length and the sense strand is 24 nucleotides in length.

In some embodiments, the antisense strand is 29 nucleotides in length and the sense strand is 25 nucleotides in length.

In some embodiments, the antisense strand is 29 nucleotides in length and the sense strand is 26 nucleotides in length.

In some embodiments, the antisense strand is 29 nucleotides in length and the sense strand is 27 nucleotides in length.

In some embodiments, the antisense strand is 29 nucleotides in length and the sense strand is 28 nucleotides in length.

In some embodiments, the antisense strand is 29 nucleotides in length and the sense strand is 29 nucleotides in length.

In some embodiments, the antisense strand is 30 nucleotides in length and the sense strand is 14 nucleotides in length.

In some embodiments, the antisense strand is 30 nucleotides in length and the sense strand is 15 nucleotides in length.

In some embodiments, the antisense strand is 30 nucleotides in length and the sense strand is 16 nucleotides in length.

In some embodiments, the antisense strand is 30 nucleotides in length and the sense strand is 17 nucleotides in length.

In some embodiments, the antisense strand is 30 nucleotides in length and the sense strand is 18 nucleotides in length.

In some embodiments, the antisense strand is 30 nucleotides in length and the sense strand is 19 nucleotides in length.

In some embodiments, the antisense strand is 30 nucleotides in length and the sense strand is 20 nucleotides in length.

In some embodiments, the antisense strand is 30 nucleotides in length and the sense strand is 21 nucleotides in length.

In some embodiments, the antisense strand is 30 nucleotides in length and the sense strand is 22 nucleotides in length.

In some embodiments, the antisense strand is 30 nucleotides in length and the sense strand is 23 nucleotides in length.

In some embodiments, the antisense strand is 30 nucleotides in length and the sense strand is 24 nucleotides in length.

In some embodiments, the antisense strand is 30 nucleotides in length and the sense strand is 25 nucleotides in length.

In some embodiments, the antisense strand is 30 nucleotides in length and the sense strand is 26 nucleotides in length.

In some embodiments, the antisense strand is 30 nucleotides in length and the sense strand is 27 nucleotides in length.

In some embodiments, the antisense strand is 30 nucleotides in length and the sense strand is 28 nucleotides in length.

In some embodiments, the antisense strand is 30 nucleotides in length and the sense strand is 29 nucleotides in length.

In some embodiments, the antisense strand is 30 nucleotides in length and the sense strand is 30 nucleotides in length. In some embodiments of any one of the foregoing aspects, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVI-XVIII:

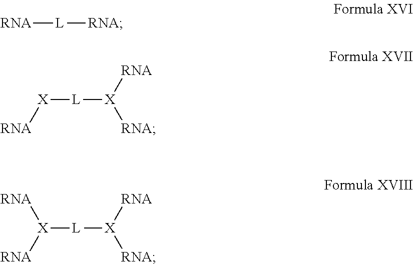

wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503). In some embodiments, the tri-branched siRNA molecule represented by any one of Formulas XIX-XXII:

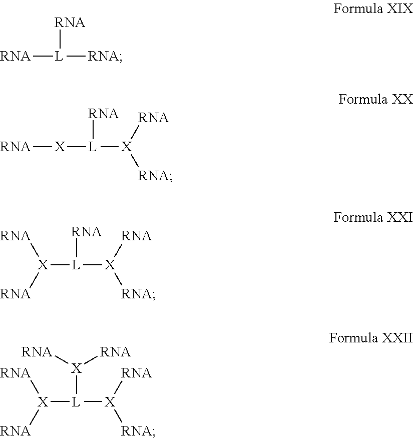

wherein each RNA, independently, is an siRNA molecule, L is a linker, and each X, independently, represents a branch point moiety. In some embodiments, the tetra-branched siRNA molecule represented by any one of Formulas XXIII-XXVII:

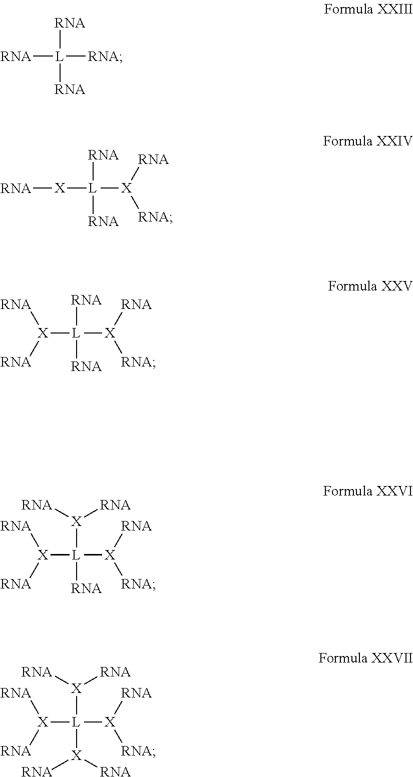

wherein each RNA, independently, is an siRNA molecule, L is a linker, and each X, independently, represents a branch point moiety.

In some embodiments, the linker is selected from a group consisting of one or more contiguous subunits of an ethylene glycol, alkyl, carbohydrate, block copolymer, peptide, RNA, and DNA. In some embodiments, the one or more contiguous subunits is 2 to 20 (e.g., 3 to 19, 4 to 18, 5 to 17, 6 to 16, 7 to 15, 8 to 14, 9 to 13, or 10 to 12) contiguous subunits.

In some embodiments, the linker is attached to one or more siRNA (e.g., 1, 2, or more) molecules of any of the foregoing aspects and embodiments by way of a covalent bond-forming moiety. In some embodiments, the covalent bond-forming moiety is selected from the group consisting of alkyl, ester, amide, carbonate, carbamate, triazole, urea, formacetal, phosphonate, phosphate, phosphorothioate, and phosphoramidate.

In some embodiments, the antisense strand has complementarity sufficient to hybridize a portion of a gene selected from ABCA7, ABI3, ADAM10, APOC1, APOE, AXL, BINI , C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

In some embodiments, the antisense strand has complementarity sufficient to hybridize a portion of a gene selected from HTT, MAPT, SNCA, C9ORF72, APOE, SCN9A, KCNT1, PRNP, and MSH3.

In some embodiments, the antisense strand has complementarity sufficient to hybridize a portion of an HTT gene.

In another aspect, the present disclosure provides a pharmaceutical composition including the siRNA molecule of any one of the foregoing aspects and embodiments, and a pharmaceutically acceptable excipient, carrier, or diluent.

In another aspect, the present disclosure provides a method of delivering an siRNA molecule to the central nervous system (CNS) of a subject, the method including administering the siRNA molecule of any one of the foregoing aspects and embodiments or the pharmaceutical composition of the foregoing aspect to the CNS of the subject. In some embodiments, the siRNA molecule or the pharmaceutical composition is administered to the subject by way of intrastriatal, intracerebroventricular, or intrathecal injection.

In some embodiments of the foregoing aspect, the delivering of the siRNA molecule to the CNS of the subject results in gene silencing of a target gene in the subject. In some embodiments, the target gene is an overactive disease driver gene. In some embodiments, the target gene is a negative regulator of a gene with reduced expression that is associated with a disease state in the subject. In some embodiments, the target gene is a positive regulator of a gene with increased expression that is associated with a disease state in a subject. In some embodiments, the target gene is a splice isoform of the target gene, wherein the splice isoform reduces expression of the target gene. In some embodiments, the gene silencing treats a disease state in the subject.

In some embodiments, the subject is a human.

In another aspect, the present disclosure provides a kit including the siRNA molecule of any one of the foregoing aspects and embodiments, or the pharmaceutical composition of the foregoing aspect, and a package insert, wherein the package insert instructs a user of the kit to perform the method of the foregoing aspect and embodiments.

### BRIEF DESCRIPTION OF THE DRAWINGS

FIGS. 1A and 1B show an antisense strand (FIG. 1A) and sense strand (FIG. 1B) of an exemplary “first generation (F1)” double-stranded (ds-) short-interfering (si) RNA (ds-siRNA) molecule (ds-siRNA A_V1) used for comparison of knockdown efficacy relative to one or more of “second generation (F2)” ds-siRNA molecules of the disclosure. The antisense and sense strands exhibit alternating patterns (i.e., motifs) of 2′-O-methyl (2′-O-Me) and 2′-fluoro (2′-F) modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 2A and 2B show an antisense strand (FIG. 2A) and sense strand (FIG. 2B) of another exemplary F1 ds-siRNA molecule (ds-siRNA A_V2) used for comparison of knockdown efficacy relative to one or more F2 ds-siRNA molecules of the disclosure. The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 7 modified ribonucleosides linked by phosphorothioate internucleoside linkages.

FIGS. 3A and 3B show an antisense strand (FIG. 3A) and sense strand (FIG. 3B) of an exemplary F2 double-stranded (ds-) short-interfering (si) RNA (ds-siRNA) molecule of the disclosure (ds-siRNA A_V3). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 4A and 4B show an antisense strand (FIG. 4A) and sense strand (FIG. 4B) of an exemplary F2 ds-siRNA molecule of the disclosure (ds-siRNA A_V4). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 5A and 5B show an antisense strand (FIG. 5A) and sense strand (FIG. 5B) of an exemplary F2 ds-siRNA molecule of the disclosure (ds-siRNA A_V5). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 6A and 6B show an antisense strand (FIG. 6A) and sense strand (FIG. 6B) of an exemplary F2 ds-siRNA molecule of the disclosure (ds-siRNA A_V6). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 7A and 7B show an antisense strand (FIG. 7A) and sense strand (FIG. 7B) of another exemplary F1 ds-siRNA molecule (ds-siRNA B_V1) used for comparison of knockdown efficacy relative to one or more F2 ds-siRNA molecules of the disclosure. The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 8A and 8B show an antisense strand (FIG. 8A) and sense strand (FIG. 8B) of another exemplary F1 ds-siRNA molecule (ds-siRNA B_V2) used for comparison of knockdown efficacy relative to one or more F2 ds-siRNA molecules of the disclosure. The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 7 modified ribonucleosides linked by phosphorothioate internucleoside linkages.

FIGS. 9A and 9B show an antisense strand (FIG. 9A) and sense strand (FIG. 9B) of an exemplary F2 ds-siRNA molecule of the disclosure (ds-siRNA B_V3). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 10A and 10B show an antisense strand (FIG. 10A) and sense strand (FIG. 10B) of an exemplary F2 ds-siRNA molecule of the disclosure (ds-siRNA B_V4). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 11A and 11B show an antisense strand (FIG. 11A) and sense strand (FIG. 11B) of an exemplary F2 ds-siRNA molecule of the disclosure (ds-siRNA B_V5). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 12A and 12B show an antisense strand (FIG. 12A) and sense strand (FIG. 12B) of an exemplary F2 ds-siRNA molecule of the disclosure (ds-siRNA B_V6). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 13A and 13B show an antisense strand (FIG. 13A) and sense strand (FIG. 13B) of an exemplary F2 ds-siRNA molecule of the disclosure (ds-siRNA C_V1). The antisense and sense strands exhibit alternating patterns (i.e., motifs) of 2′-O-Me and 2′-F modified ribonucleosides. The ds-sRNA molecule also features a central block of about 11 to 13 modified ribonucleosides linked by phosphodiester internucleoside linkages flanked on the 5′ end and the 3′ end, or on the 5′ end only, by a block of 2 to 5 modified ribonucleosides linked by phosphorothioate internucleoside linkages.

FIGS. 14A-14F are a series of scatter plots showing expression levels of huntingtin (HTT) mRNA level normalized to the housekeeping gene ATP5b in different brain regions (i.e., hippocampus, cortex, and striatum) of FVB/NJ female mice treated with varying doses (i.e., 0.2 nmol, 1.0 nmol, or 5.0 nmol) of an exemplary di-siRNA of the disclosure, including F1 molecules ds-siRNA A_V1 (FIG. 14A) and ds-siRNA A_V2 (FIG. 14B), and F2 molecules ds-siRNA A_V3 (FIG. 14C), ds-siRNA A_V4 (FIG. 14D), ds-siRNA A_V5 (FIG. 14E), and ds-siRNA A_V6 (FIG. 14F), or a vehicle control (PBS) administered via intracerebroventricular (ICV) injection.

FIGS. 15A-15F are a series of scatter plots showing expression levels of HTT mRNA level normalized to the housekeeping gene ATP5b in different brain regions (i.e., hippocampus, cortex, and striatum) of FVB/NJ female mice treated with varying doses (i.e., 0.2 nmol, 1.0 nmol, or 5.0 nmol) of an exemplary di-siRNA of the disclosure, including F1 molecules ds-siRNA B_V1 (FIG. 15A) and ds-siRNA B_V2 (FIG. 15B), and F2 molecules ds-siRNA B_V3 (FIG. 15C), ds-siRNA B_V4 (FIG. 15D), ds-siRNA B_V5 (FIG. 15E), and ds-siRNA B_V6 (FIG. 15F), or a vehicle control (PBS) administered via ICV injection.

FIG. 16 is a scatter plot showing expression levels of HTT mRNA level normalized to the housekeeping gene ATP5b in different brain regions (i.e., hippocampus, cortex, and striatum) of FVB/NJ female mice treated with varying doses (i.e., 0.2 nmol, 1.0 nmol, or 5.0 nmol) of an exemplary di-siRNA of the disclosure, namely ds-siRNA C_V1, or a vehicle control (PBS) administered via ICV injection.

FIGS. 17A and 17B are graphs showing expression levels of HTT mRNA level normalized to the housekeeping gene ATP5b in different brain regions (i.e., hippocampus, cortex, and striatum) of FVB/NJ female mice treated with doses of 0.5 nmol (FIG. 17A) and 2.5 nmol (FIG. 17B) of an exemplary di-siRNA of the disclosure, including F1 molecules ds-siRNA A_V1, ds-siRNA A_V2, ds-siRNA B_V1, and ds-siRNA B_V2, and F2 molecules ds-siRNA A_V3, ds-siRNA A_V4, ds-siRNA A_V5, ds-siRNA A_V6 ds-siRNA B_V3, ds-siRNA B_V4, ds-siRNA B_V5, ds-siRNA B_V6, and ds-siRNA C_V1, or a vehicle control (PBS) administered via ICV injection.

FIG. 18 is a scatter plot showing the toxicity profile of an exemplary di-siRNA of the disclosure, including F1 molecule ds-siRNA A_V1 and F2 molecules ds-siRNA A_V3, ds-siRNA A_V4, ds-siRNA B_V3, ds-siRNA B_V4, ds-siRNA B_V6, and ds-siRNA C_V1 as quantified by the EvADINT scoring assay. A higher score indicates a greater level of toxicity.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively. As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group on its 3′ or 5′ sugar hydroxyl group.

As used herein, the term “siRNA” refers to small interfering RNA duplexes that induce the RNA interference (RNAi) pathway. siRNA molecules can vary in length (generally, between 18 and 30 base pairs) and contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

As used herein, the terms “chemically modified nucleotide” or “nucleotide analog” or “altered nucleotide” or “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified from 2′-hydroxyl groups to 2′-O-methyl groups.

As used herein, the term “phosphorothioate” refers to a phosphate group of a nucleotide that is modified by substituting one or more of the oxygens of the phosphate group with sulfur.

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and ted-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group can be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl-CH₂—. A benzyl group can be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “internucleoside” and “internucleotide” refer to the bonds between nucleosides and nucleotides, respectively.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, the term “lipophilic amino acid” refers to an amino acid including a hydrophobic moiety (e.g., an alkyl chain or an aromatic ring).

As used herein, the term “antagomiRs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that are comprised of a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “target of delivery” refers to the organ or part of the body that is desired to deliver the branched oligonucleotide compositions to.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety can be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group can include from 1 to 3 phosphate moieties that are each, independently, unmodified (di- or tri-phosphates) or modified.

As used herein, the term “between X and Y” is inclusive of the values of X and Y. For example, “between X and Y” refers to the range of values between the value of X and the value of Y, as well as the value of X and the value of Y.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

It is understood that certain internucleoside linkages provided herein, including, e.g., phosphodiester and phosphorothioate, include a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., transgene, heterologous gene and/or endogenous gene expression, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner via RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

The term “negative regulator,” as used herein, refers to a gene that negatively regulates (e.g., reduces or inhibits) the expression and/or activity of another gene or set of genes.

The term “positive regulator,” as used herein, refers to a gene that positively regulates (e.g., increases or saturates) the expression and/or activity of another gene or set of genes.

The term “phosphate moiety” as used herein, refers to a terminal phosphate group that includes phosphates as well as modified phosphates. The phosphate moiety can be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group can include from 1 to 3 phosphate moieties that are each, independently, unmodified (di or tri-phosphates) or modified.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

### DETAILED DESCRIPTION

The present invention provides new forms of siRNA, such as single-stranded (ss-) or double-stranded short interfering RNA (ds-siRNA) molecules having specific patterns of chemical modifications (e.g., 2′ ribose modifications or internucleoside linkage modifications) to improve resistance against nuclease enzymes, toxicity profile, and physicochemical properties (e.g., thermostability). The siRNA compositions of the disclosure may employ a variety of modifications previously known and/or unknown in the art. In addition, the present disclosure features branched siRNA structures, such as di-branched, tri-branched, and tetra-branched ds-siRNA structures.

The siRNA of the disclosure may contain an antisense strand including a region that is represented by Formula I:

A-B-(A′)ⱼ-C-P²-D-P¹-(C′-P¹)ₖ-C′ Formula I;

wherein A is represented by the formula C-P¹-D-P¹; each A′, independently, is represented by the formula C-P²-D-P²; B is represented by the formula C-P²-D-P²-D-P²-D-P²; each C, independently, is a 2′-O-methyl (2′-O-Me) ribonucleoside; each C′, independently, is a 2′-O-Me ribonucleoside or a 2′-fluoro (2′-F) ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7; and k is an integer from 1 to 7.

In some embodiments, the antisense strand includes a structure represented by formula A1, wherein Formula A1 is, in the 5′-to-3′ direction:

The siRNA of the disclosure may contain an antisense strand including a region that is represented by Formula II:

A-B-(A′)ⱼ-C-P²-D-P¹-(C-P¹)ₖ-C′ Formula II;

wherein A is represented by the formula C-P¹-D-P¹; each A′, independently, is represented by the formula C-P²-D-P²; B is represented by the formula C-P²-D-P²-D-P²-D-P²; each C, independently, is a 2′-O-methyl (2′-O-Me) ribonucleoside; each C′, independently, is a 2′-O-Me ribonucleoside or a 2′-fluoro (2′-F) ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the siRNA of the disclosure may have a sense strand represented by

E-(A′)ₘ-F Formula III;

wherein E is represented by the formula (C-P¹)₂; F is represented by the formula (C-P²)₃-D-P¹-C-P¹-C, (C-P²)₃-D-P²-C-P²-C, or (C-P²)₃-D-P²-C-P²-D; A′, C, D, P¹, and P² are as defined in Formula II; and m is an integer from 1 to 7 (e.g., 1, 2, 3, 4, 5, 6, or 7). In some embodiments, m is 4. The sense strand is complementary (e.g., fully or partially complementary) to the antisense strand.

Alternatively, the siRNA of the disclosure may contain an antisense strand including a region that is represented by Formula IV:

A-(A′)ⱼ-C-P²-B-(C-P¹)ₖ-C′ Formula IV;

wherein A is represented by the formula C-P¹-D-P¹; each A′, independently, is represented by the formula C-P²-D-P²; B is represented by the formula D-P¹-C-P¹-D-P¹; each C, independently, is a 2′-O-Me ribonucleoside; each C′, independently, is a 2′-O-Me ribonucleoside or a 2′-F ribonucleoside; each D, independently, is a 2′-F ribonucleoside; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); and k is an integer from 1 to7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments, the siRNA of the disclosure may have a sense strand represented by Formula V:

E-(A′)ₘ-C-P²-F Formula V;

wherein E is represented by the formula (C-P¹)₂; F is represented by the formula D-P¹-C-P¹C, D-P²-C-P²C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

Alternatively, the siRNA of the disclosure may contain an antisense strand including a region that is represented by Formula VI:

A-Bⱼ-E-Bₖ-E-F-Gₗ-D-P¹-C′ Formula VI;

wherein A is represented by the formula C-P¹-D-P¹; each B, independently, is represented by the formula C-P²; each C, independently, is a 2′-O-Me ribonucleoside; each C′, independently, is a 2′-O-Me ribonucleoside or a 2′-F ribonucleoside; each D, independently, is a 2′-F ribonucleoside; each E, independently, is represented by the formula D-P²-C-P²; F is represented by the formula D-P¹-C-P¹; each G, independently, is represented by the formula C-P¹; each P¹ is, independently, a phosphorothioate internucleoside linkage; each P² is, independently, a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and I is an integer from 1 to 7 (e.g., 1, 2, 3, 4, 5, 6, or 7). In some embodiments, j is 3. In some embodiments, k is 6. In some embodiments, l is 2. In some embodiments, j is 3, k is 6, and l is 2. The antisense strand is complementary (e.g., fully or partially complementary) to a target nucleic acid.

In some embodiments, the siRNA of the disclosure may have a sense strand represented by Formula VII:

H-Bₘ-Iₙ-A′-Bₒ-H-C Formula VII;

wherein A′ is represented by the formula C-P²-D-P²; each H, independently, is represented by the formula (C-P¹)₂; each I, independently, is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

siRNA Structure

The simplest siRNAs consist of a ribonucleic acid including a ss- or ds- structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNA interference (RNAi) activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence can be between 80% and 100%, e.g., 80%, 85%, 90%, 95%, or 100% complementary.

siRNAs described herein may employ modifications to the nucleobase, phosphate backbone, ribose core, 5′- and 3′-ends, and branching, wherein multiple strands of siRNA may be covalently linked.

Length of siRNA Molecules

It is within the scope of the disclosure that any length, known and previously unknown in the art, may be employed for the current invention. As described herein, potential lengths for an antisense strand of the branched siRNA of the present invention is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the branched siRNA of the present invention is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present invention includes ss- and ds-siRNA compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Some embodiments use O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O-CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), -O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

Oligomeric compounds may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present invention. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. Oligomeric compounds of the present invention can also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-l,3-diazaphenoxazine-2-one (Wang et al., Tetrahedron Left., 39:8385-8, 1998). Incorporated into oligonucleotides these base modifications were shown to hybridize with complementary guanine and the latter was also shown to hybridize with adenine and to enhance helical thermal stability by extended stacking interactions (also see U.S. Pat. Nos. 10/155,920 and US 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present invention are the internucleoside linkages making up the phosphate backbone. Although the natural RNA phosphate backbone may be employed here, derivatives thereof, known and yet unknown in the art, may be used which enhance desirable characteristics of a siRNA. Although not limiting, of particular importance in the present invention is protecting parts, or the whole, of the siRNA from hydrolysis. One example of a modification that decreases the rate of hydrolysis is phosphorothioates. Any portion or the whole of the backbone may contain phosphate substitutions (e.g., phosphorothioates, phosphodiesters, etc.). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70% 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages. Specific examples of some potential oligomeric compounds useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

siRNA Patterning

Nucleosides used in the invention feature a range of modifications in the nucleobase and sugar. A complete ss- or ds-siRNA may have 1, 2, 3, 4, 5, or more different nucleosides that each appear in the siRNA strand or strands once or more. The nucleosides may appear in a repeating pattern (e.g., alternating between two modified nucleosides) or may be a strand of one type of nucleoside with substitutions of a second type of nucleoside. Similarly, internucleoside linkages may be of one or more type appearing in a single- or double-stranded siRNA in a repeating pattern (e.g., alternating between two internucleoside linkages) or may be a strand of one type of internucleoside linkage with substitutions of a second type of internucleoside linkage. Though the siRNAs of the disclosure tolerate a range of substitution patterns, the following exemplify some preferred patterns of siRNA modifications in the antisense strand of a single-stranded or ds-siRNA molecule, in which A and B represent nucleosides of two types, and S and O represent internucleoside linkages of two types:

Antisense Pattern 1:

A-S-B-S-A-O-B-O-B-O-B-O-A-O-B-O-A-O-B-O-A-O-B-O-A-O-B-O-A-O-B-S-A-S-A-S-A-S-B-S-A (Formula A1)

Antisense Pattern 2:

A-S-B-S-A-O-B-O-B-O-B-O-A-O-B-O-A-O-B-O-A-O-B-O-A-O-B-O-A-O-B-S-A-S-A-S-A-S-A-S-A (Formula A2)

Antisense Pattern 3:

A-S-B-S-A-O-B-O-A-O-B-O-A-O-B-O-A-O-B-O-A-O-B-O-A-O-B-O-A-O-B-S-A-S-B-S-A-S-A-S-A (Formula A3)

Antisense Pattern 4:

A-S-B-S-A-O-A-O-A-O-B-O-A-O-A-O-A-O-A-O-A-O-A-O-A-O-B-O-A-O-B-S-A-S-A-S-A-S-B-S-A (Formula A4)

Non-limiting examples of patterns of siRNA modifications in the sense strand of a ds-siRNA molecule are shown below:

### Sense Patterns Compatible With Antisense Pattern 1 or 2:

Sense Pattern 1:

A-S-A-S-A-O-B-O-A-O-B-O-A-O-B-O-A-O-B-O-A-O-A-O-A-O-B-S-A-S-A (Formula S1)

Sense Pattern 2:

A-S-A-S-A-O-B-O-A-O-B-O-A-O-B-O-A-O-B-O-A-O-A-O-A-O-B-O-A-O-A (Formula S2)

Sense Pattern 3:

A-S-A-S-A-O-B-O-A-O-B-O-A-O-B-O-A-O-B-O-A-O-A-O-A-O-B-S-A-S-B (Formula S3)

Sense Pattern 4:

A-S-A-S-A-O-B-O-A-O-B-O-A-O-B-O-A-O-B-O-A-O-A-O-A-O-B-O-A-O-B (Formula S4)

### Sense Patterns Compatible With Antisense Pattern 3:

Sense Pattern 5:

A-S-A-S-A-O-B-O-A-O-B-O-A-O-B-O-A-O-B-O-A-O-B-O-A-O-B-S-A-S-A (Formula S5)

Sense Pattern 6:

A-S-A-S-A-O-B-O-A-O-B-O-A-O-B-O-A-O-B-O-A-O-B-O-A-O-B-O-A-O-A (Formula S6)

Sense Pattern 7:

A-S-A-S-A-O-B-O-A-O-B-O-A-O-B-O-A-O-B-O-A-O-B-O-A-O-B-S-A-S-B (Formula S7)

Sense Pattern 8:

A-S-A-S-A-O-B-O-A-O-B-O-A-O-B-O-A-O-B-O-A-O-B-O-A-O-B-O-A-O-B (Formula S8)

Sense Patterns Compatible with Antisense Pattern 4:

Sense Pattern 9:

A-S-A-S-A-O-A-O-A-O-B-O-B-O-B-O-A-O-B-O-A-O-A-O-A-O-A-S-A-S-A (Formula S9)

In some embodiments, A represents a 2′-O-Me nucleoside. In some embodiments, B represents a 2′-F nucleoside. In some embodiments, O represents a phosphodiester internucleoside linkage. In some embodiments, S represents a phosphorothioate internucleoside linkage.

### 5′ Phosphorus Stabilizing Moiety

To further protect the siRNA from degradation a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety.

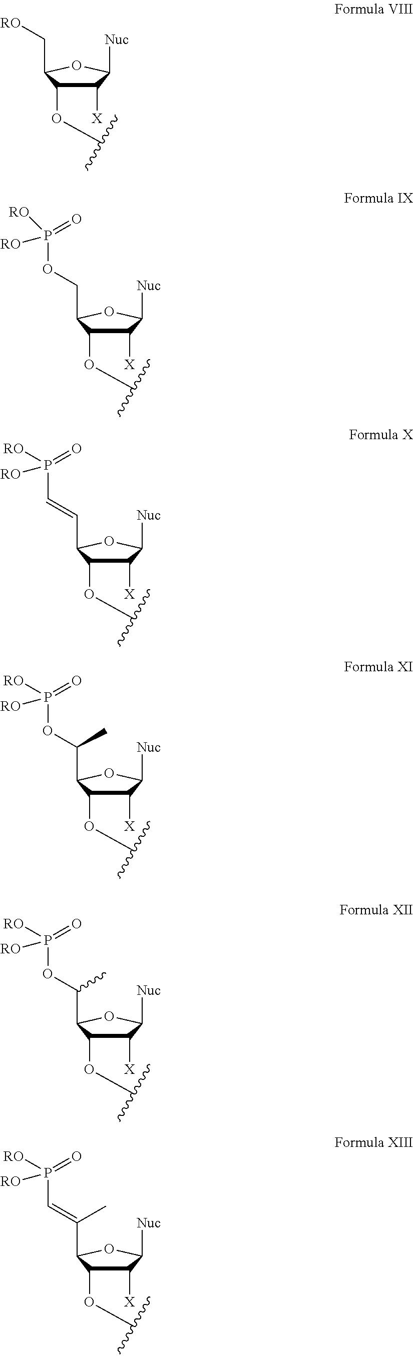

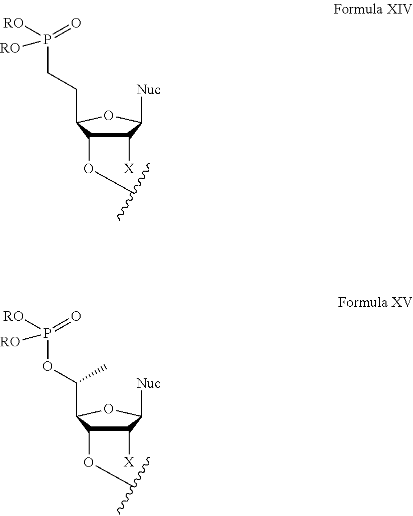

Some exemplary endcaps are demonstrated in Formulas VIII-XV. Nuc in Formulas VIII-XV represents a nucleobase or nucleobase derivative or replacement as described herein. X in formula VIII-XV represents a 2′-modification as described herein. Some embodiments employ hydroxy as in Formula VIII, phosphate as in Formula IX, vinylphosphonates as in Formula X and XIII, 5′-methyl-substituted phosphates as in Formula XI, XIII, and XV, or methylenephosphonates as in Formula XIV. Vinyl 5′-vinylphsophonate as a 5′-phosphorus stabilizing moiety as demonstrated in Formula X.

### Hydrophobic Moieties

The present disclosure further provides siRNA molecules having one or more hydrophobic moieties attached thereto. The hydrophobic moiety may be covalently attached to the 5′ end or the 3′ end of an siRNA molecule of the disclosure. Non-limiting examples of hydrophobic moieties suitable for use with the siRNAs of the disclosure include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

siRNA Branching

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 1.

TABLE 1
Branched siRNA structures

| Di-branched | Tri-branched | Tetra-branched |
|---|---|---|
| RNA—L—RNA Formula XVI | 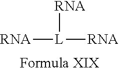 | 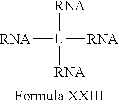 |
| 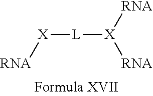 | 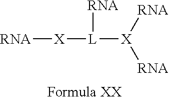 | 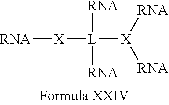 |
| 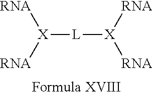 | 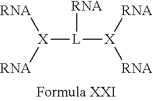 | 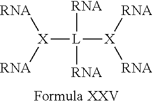 |
| | 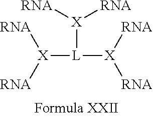 | 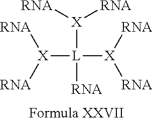 |
| | | 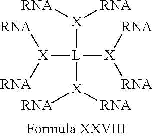 |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVI-XVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XIX-XXII, wherein each RNA, independently, is an siRNA molecule, L is a linker, and each X, independently, represents a branch point moiety.

In some embodiments, the tetra-branched siRNA molecule represented by any one of Formulas XXIII-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene linker (TEG).

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1, as is shown below:

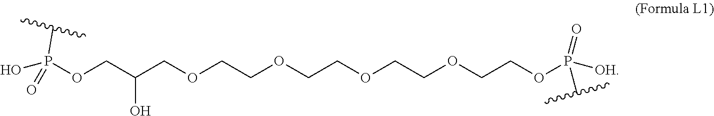 [SMILES: *P(=O)(O)OCCOCCOCCOCCOCC(O)COP(*)(=O)O]

In some embodiments, the linker has a structure of Formula L2, as is shown below:

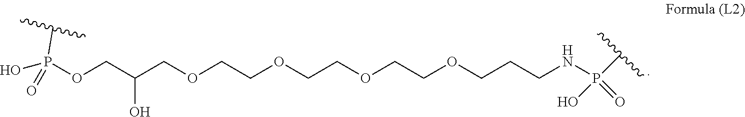 [SMILES: *P(=O)(O)NCCCOCCOCCOCCOCC(O)COP(*)(=O)O]

In some embodiments, the linker has a structure of Formula L3, as is shown below:

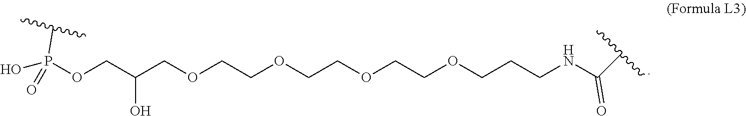 [SMILES: *C(=O)NCCCOCCOCCOCCOCC(O)COP(*)(=O)O]

In some embodiments, the linker has a structure of Formula L4, as is shown below:

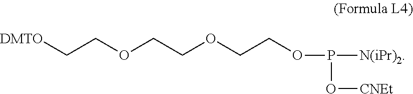

In some embodiments, the linker has a structure of Formula L5, as is shown below:

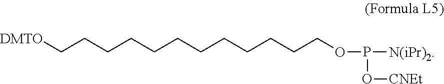

In some embodiments, the linker has a structure of Formula L6, as is shown below:

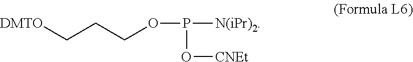

In some embodiments, the linker has a structure of Formula L7, as is shown below:

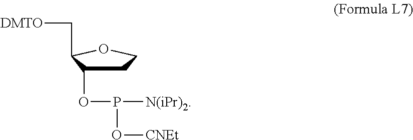

In some embodiments, the linker has a structure of Formula L8, as is shown below:

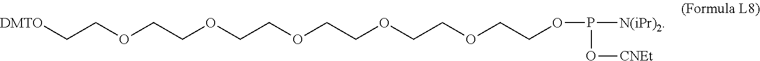

In some embodiments, the linker has a structure of Formula L9, as is shown below:

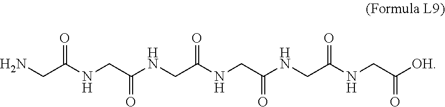 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein can be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Genes

The siRNA molecules of the disclosure may include an antisense strand and sense strand having complementarity to the antisense strand, wherein the antisense strand is from 10 to 30 nucleotides in length and has complementarity sufficient to hybridize to a region of any of the following genes: ABCA7, AB13, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1. In some embodiments, the antisense strand has complementarity sufficient to hybridize to a region of any of the following genes: APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL1 ORA, IL1A, IL1B, IL1RAP, INPP5D, ITGAM, MEF2C, MMP12, NLRP3, NOS2, PILRA, PLCG2, PTK2B, SLC24A4, TBK1, and TNF. In some embodiments, the antisense strand has complementarity to sufficient to hybridize to a region of any of the following genes: HTT, MAPT, SNCA, C9ORF72, APOE, SCN9A, KCNT1, PRNP, and MSH3. In some embodiments, the siRNA molecule has complementarity sufficient to hybridize to a region of an HTT gene.

### Methods of Treatment

The invention provides methods of treating a subject in need of gene silencing. The gene silencing may be performed in order to silence defective or overactive genes, silence negative regulators of genes with reduced expression, silence wild type genes with an activating role in a pathway(s) that increases activity of a disease driver gene, silence splice isoforms of a gene(s) that, when selectively knocked down, may elevate total expression of the gene(s), among other reasons, so long as the goal is to restore genetic and biochemical pathway activity from a disease state towards a healthy state. The method may include delivering to the CNS of the subject (e.g., a human) an siRNA molecule of the disclosure or a pharmaceutical composition containing the same by any appropriate route of administration (e.g., intrastriatal, intracerebroventricular, or intrathecal injection). The active compound can be administered in any suitable dose. The actual dosage amount of a composition of the present invention administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Diseases

The subject in need of gene silencing may be in need of silencing of a gene found in the CNS (e.g., in a microglial cell). The gene may be associated with a specific disease or disorder. For example, the gene may be associated with Huntington's disease, Parkinson's disease, Alzheimer's disease, amyotrophic lateral sclerosis (ALS), dementia with Lewy bodies (DLB), pure autonomic failure, Lewy body dysphagia, incidental Lewy body disease (ILBD), inherited Lewy body disease, olivopontocerebellar atrophy (OPCA), striatonigral degeneration, Shy-Drager syndrome, epilepsy or an epilepsy disorder, a prion disease, or pain or a pain disorder.

### Genes

The methods of gene silencing described herein may be performed in order to silence defective or overactive genes, silence negative regulators of genes with reduced expression, silence wild type genes with an activating role in a pathway(s) that increases activity of a disease driver gene, silence splice isoforms of a gene(s) that, when selectively knocked down, may elevate total expression of the gene(s), among other reasons, so long as the goal is to restore genetic and biochemical pathway activity from a disease state towards a healthy state.

The disease or disorder may be associated with any of the following genes: ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL1 ORA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1. In some embodiments, the disease or disorder is associated with any of the following genes: APOE, BIN1, C1 QA, C3, C9ORF72, CCL5, CD33, CLU/APOJ, CR1, CXCL10, CXCL13, IFIT1, IFIT3, IFITM3, IFNAR1, IFNAR2, IL1 ORA, IL1A, IL1B, IL1RAP, INPP5D, ITGAM, MEF2C, MMP12, NLRP3, NOS2, PILRA, PLCG2, PTK2B, SLC24A4, TBK1, and TNF. In some embodiments, the disease or disorder is associated with any of the following genes: HTT, MAPT, SNCA, C9ORF72, APOE, SCN9A, KCNT1, PRNP, and MSH3. In some embodiments, the disease or disorder is associated with an HTT gene.

### Pharmaceutical Compositions

The branched siRNA molecules in the present invention can be formulated into a pharmaceutical composition for administration to a subject in a biologically compatible form suitable for administration in vivo. Accordingly, the present disclosure provides a pharmaceutical composition containing an siRNA of the disclosure in admixture with a suitable diluent, carrier, or excipient. The siRNA can be administered, for example, directly into the CNS of the subject (e.g., by way of intrastriatal, intracerebroventricular, or intrathecal injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Regimens

A physician having ordinary skill in the art can readily determine an effective amount of siRNA for administration to a mammalian subject (e.g., a human) in need thereof. For example, a physician could start prescribing doses of a siRNA of the disclosure at levels lower than that required in order to achieve the desired therapeutic effect and gradually increase the dosage until the desired effect is achieved. Alternatively, a physician may begin a treatment regimen by administering a siRNA at a high dose and subsequently administer progressively lower doses until a therapeutic effect is achieved (e.g., a reduction in expression of a target gene sequence). In general, a suitable daily dose of a siRNA of the disclosure will be an amount of the siRNA which is the lowest dose effective to produce a therapeutic effect. A single-strand or double-strand siRNA molecule of the disclosure may be administered by injection, e.g., intrathecally, intracerebroventricularly, or intrastriatally (e.g., injection into the caudate nucleus or putamen). A daily dose of a therapeutic composition of a siRNA of the disclosure may be administered as a single dose or as two, three, four, five, six or more doses administered separately at appropriate intervals throughout the day, week, month, or year, optionally, in unit dosage forms. While it is possible for a siRNA of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, or intrastriatally.

Intrathecal injection is the direct injection into the spinal column or subarachnoid space. By injecting directly into the CSF of the spinal column the siRNA molecule of the disclosure has direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1: Method of Preparing a Double-Stranded Short-Interfering RNA Molecule Having Patterned Ribonucleoside Modifications and Internucleoside Linkage Modifications (I)

A double-stranded (ds-) short-interfering (si) RNA (ds-siRNA) molecule having patterned ribonucleoside modifications and internucleoside linkage modifications of the disclosure is prepared according to methods well-known in the art, such as methods disclosed herein. The ds-siRNA molecule is a duplex oligoribonucleotide in which the sense strand is derived from and has full (i.e., 100%) or partial (e.g., at least 70%, 75%, 80%, 85%, 90%, 95%, 96%, 97%, 98%, 99%, or more) sequence identity to an mRNA sequence of a target gene. The nucleic acid sequence of the antisense strand is fully (i.e., 100%) complementary or partially (e.g., at least 70%, 75%, 80%, 85%, 90%, 95%, 96%, 97%, 98%, 99%, or more) complementary to the nucleic acid sequence of the sense strand/mRNA of target gene. The antisense and sense strands of the ds-siRNA agent are each synthesized according to established methods (e.g., synthesis and ligation or tandem synthesis) to include alternating patterns (i.e., motifs) of modified ribonucleosides, such as 2′-O-methyl (2′-O-Me) and 2′-fluoro (2′-F) ribonucleosides and modified internucleoside linkages, such as phosphorothioate linkages. The antisense strand is produced to be of a desirable length such that a functional benefit (e.g., RNA interference, thermal stability, and/or resistance against nucleases) is achieved. An exemplary antisense strand may be, e.g., 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. The sense strand is produced to be of a desirable length such that a functional benefit (e.g., efficient RISC loading, thermal stability, and/or resistance against nucleases) is achieved. An exemplary sense strand may be, e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. Consequent to the difference in length between the antisense strand and the sense strand, the ds-siRNA duplex structure contains a 5′ overhang, 3′ overhang, or both. An exemplary antisense strand may have the following pattern:

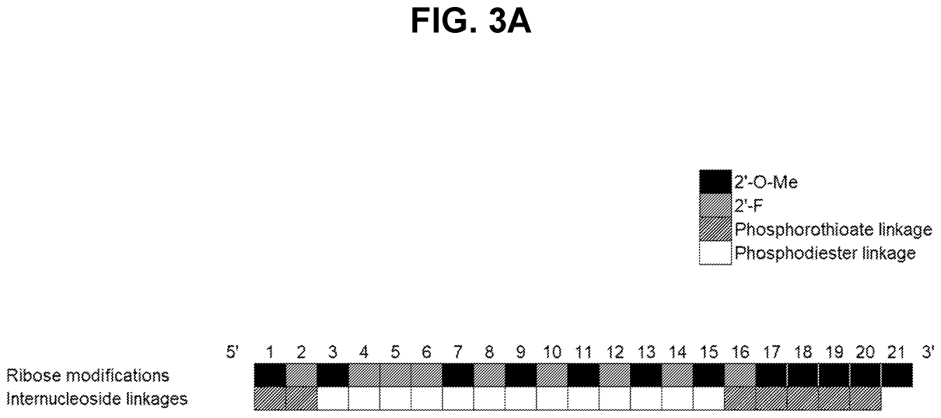

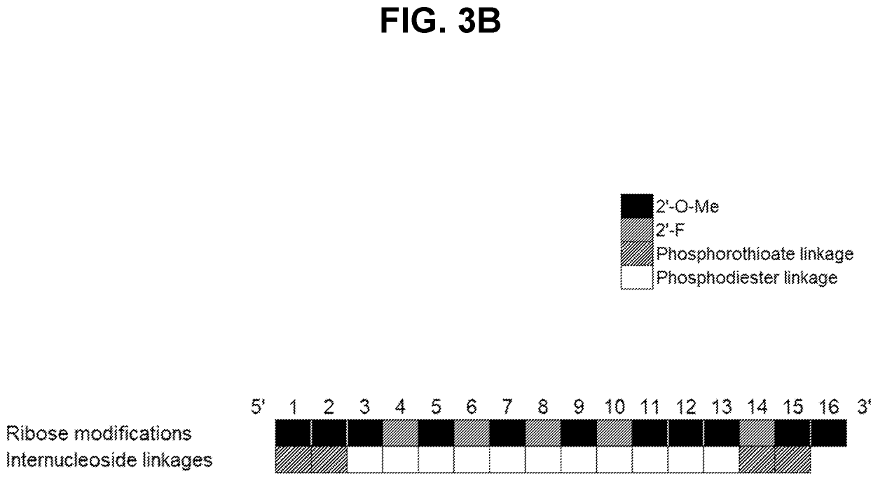

A-S-B-S-A-O-B-O-B-O-B-O-A-O-B-O-A-O-B-O-A-O-B-O-A-O-B-O-A-O-B-S-A-S-A-S-A-S-A-S-A (FIGS. 3A, 4A, 5A, and 6A); Antisense Pattern 2 (Formula A2):

An exemplary sense strand may have any one of the following patterns:

A-S-A-S-A-O-B-O-A-O-B-O-A-O-B-O-A-O-B-O-A-O-A-O-A-O-B-S-A-S-A (FIG. 3B); Sense Pattern 1 (Formula S1):

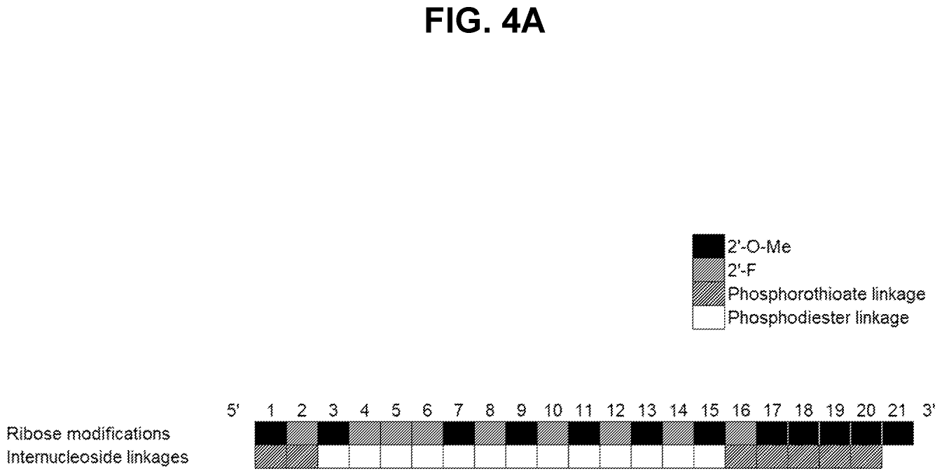

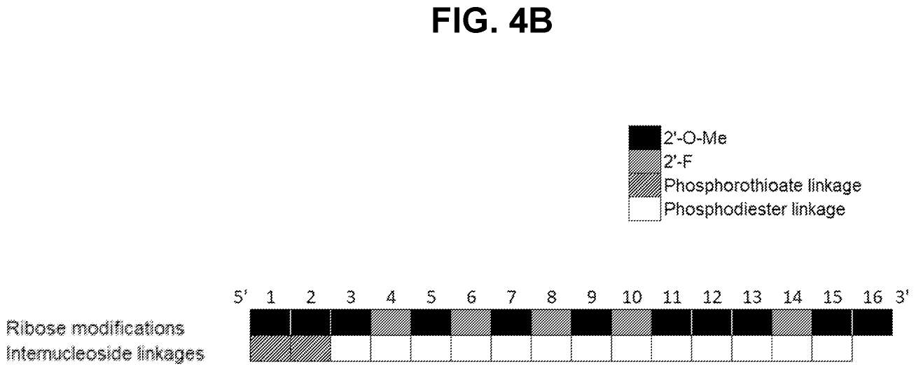

A-S-A-S-A-O-B-O-A-O-B-O-A-O-B-O-A-O-B-O-A-O-A-O-A-O-B-O-A-O-A (FIG. 4B) Sense Pattern 2 (Formula S2):

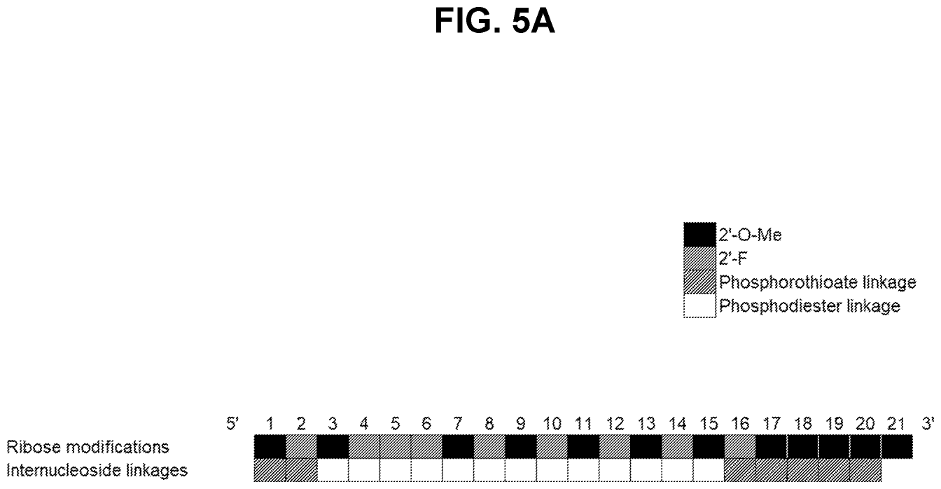

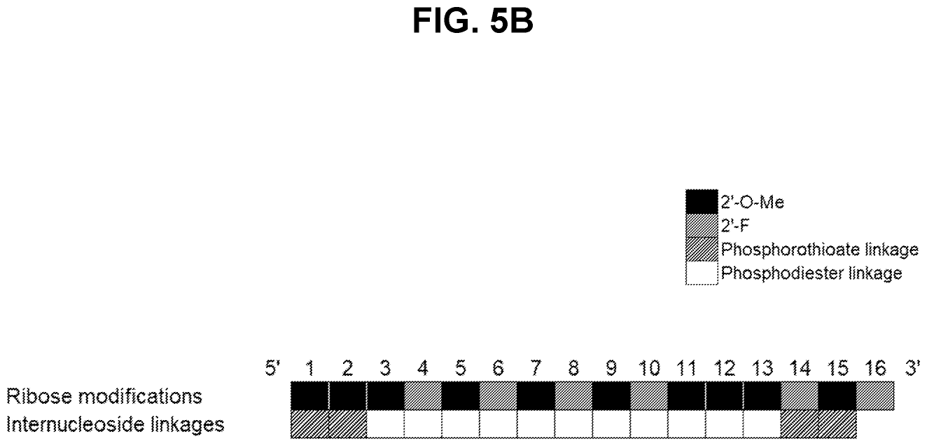

A-S-A-S-A-O-B-O-A-O-B-O-A-O-B-O-A-O-B-O-A-O-A-O-A-O-B-S-A-S-B (FIG. 5B); Sense Pattern 3 (Formula S3):

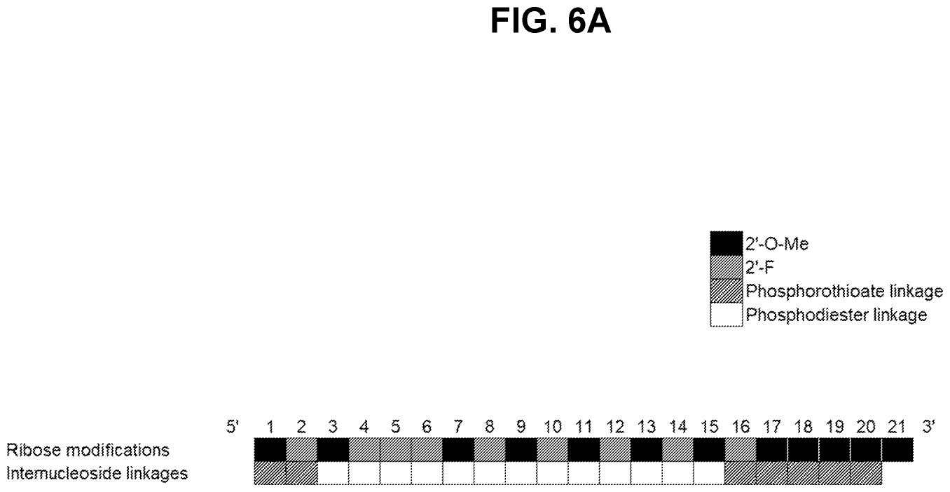

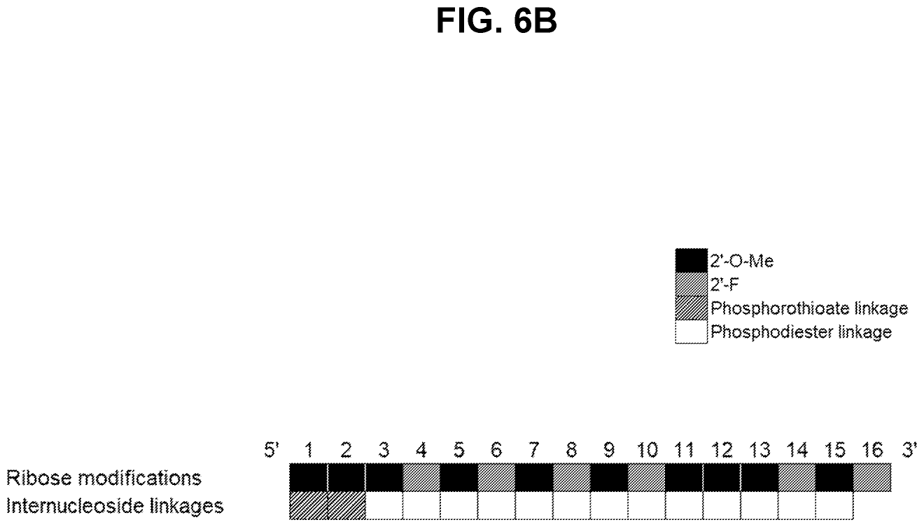

A-S-A-S-A-O-B-O-A-O-B-O-A-O-B-O-A-O-B-O-A-O-A-O-A-O-B-O-A-O-B (FIG. 6B) Sense Pattern 4 (Formula S4):

wherein A represents a 2′-O-Me ribonucleoside, B represents a 2′-F ribonucleoside, O represents a phosphodiester internucleoside linkage, and S represents a phosphorothioate internucleoside linkage.

The antisense and sense strand of the ds-siRNA molecule are each produced such that the resulting duplex structure has 0 to 5 nucleotide mismatches (e.g., 0, 1, 2, 3, 4, or 5) mismatches between the sense strand and the antisense strand and/or the antisense strand and the target mRNA sequence. The ds-siRNA molecule may be further modified to incorporate a 5′ phosphorus stabilizing moiety (e.g., a 5′-vinylphosphonate) and/or a hydrophobic moiety (e.g., cholesterol, vitamin D, or tocopherol) on the antisense strand, sense strand, or both. In addition, the ds-siRNA molecule may contain branched structures disclosed herein, such as di-branched, tri-branched, or tetra-branched structures disclosed herein. The ds-siRNA agent may be further incorporated into a pharmaceutical composition containing a pharmaceutically acceptable excipient, carrier, or diluent.

### Example 2: Method of Preparing a ds-siRNA Molecule Having Patterned Ribonucleoside Modifications and Internucleoside Linkage Modifications (II)

A ds-siRNA molecule having patterned ribonucleoside modifications and internucleoside linkage modifications of the disclosure is prepared according to methods well-known in the art, such as methods disclosed herein. The ds-siRNA molecule is a duplex oligoribonucleotide in which the sense strand is derived from and has full (i.e., 100%) or partial (e.g., at least 70%, 75%, 80%, 85%, 90%, 95%, 96%, 97%, 98%, 99%, or more) sequence identity to an mRNA sequence of a target gene. The nucleic acid sequence of the antisense strand is fully (i.e., 100%) complementary or partially (e.g., at least 70%, 75%, 80%, 85%, 90%, 95%, 96%, 97%, 98%, 99%, or more) complementary to the nucleic acid sequence of the sense strand/mRNA of target gene. The antisense and sense strands of the ds-siRNA agent are each synthesized according to well-known methods (e.g., synthesis and ligation or tandem synthesis) to contain alternating patterns (i.e., motifs) of modified ribonucleosides, such as 2′-O-Me and 2′-F ribonucleosides and modified internucleoside linkages, such as phosphorothioate linkages. The antisense strand is produced to be of a desirable length such that a functional benefit (e.g., RNA interference, thermal stability, and/or resistance against nucleases) is achieved. An exemplary antisense strand may be, e.g., 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. The sense strand is produced to be of a desirable length such that a functional benefit (e.g., efficient RISC loading, thermal stability, and/or resistance against nucleases) is achieved. An exemplary sense strand may be, e.g., 15 nucleotides, 16 nucleotides, 17 nucleotides, 18 nucleotides, 19, nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. Consequent to the difference in length between the antisense strand and the sense strand, the ds-siRNA duplex structure contains a 5′ overhang, 3′ overhang, or both. An exemplary antisense strand may have the following pattern:

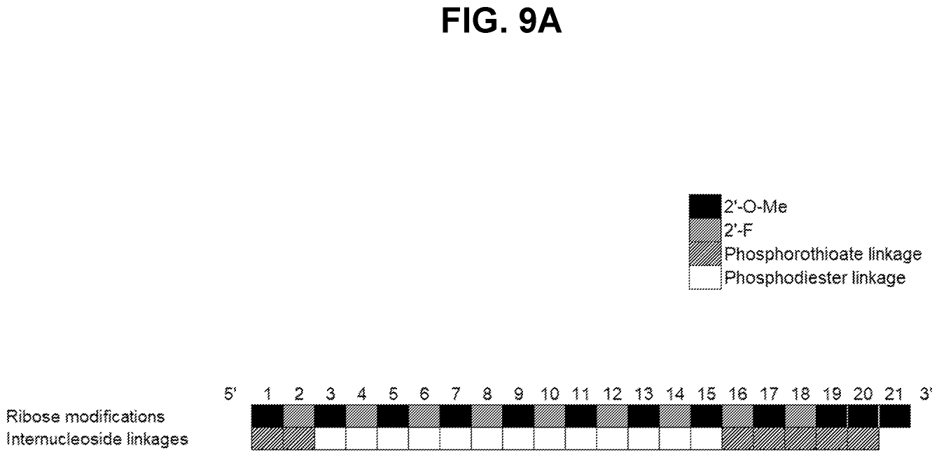

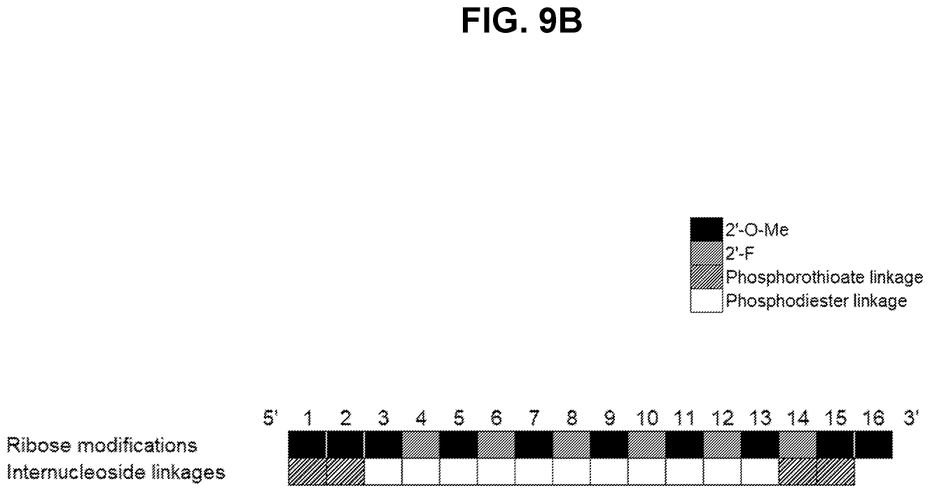

A-S-B-S-A-O-B-O-A-O-B-O-A-O-B-O-A-O-B-O-A-O-B-O-A-O-B-O-A-O-B-S-A-S-B-S-A-S-A-S-A (FIGS. 9A, 10A, 11A, and 12A); Antisense Pattern 3 (Formula A3):

An exemplary sense strand may have any one of the following patterns:

A-S-A-S-A-O-B-O-A-O-B-O-A-O-B-O-A-O-B-O-A-O-B-O-A-O-B-S-A-S-A (FIG. 9B); Sense Pattern 5 (Formula S5):

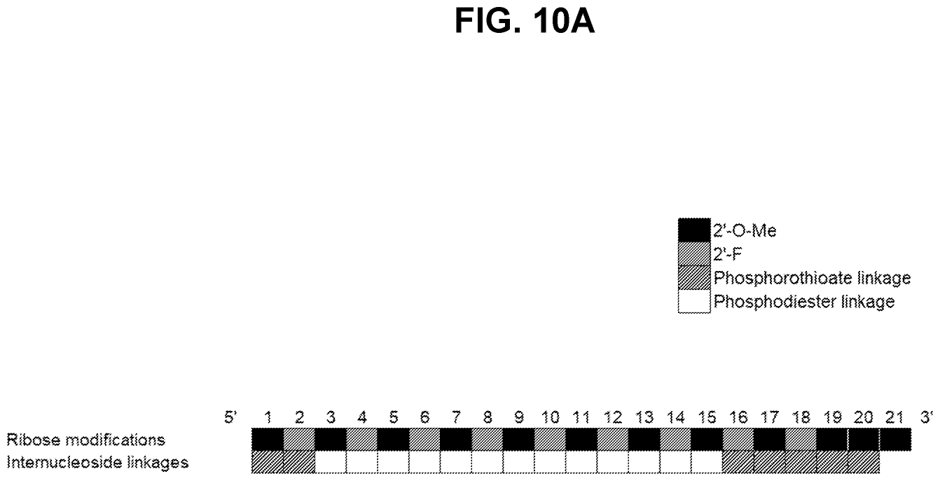

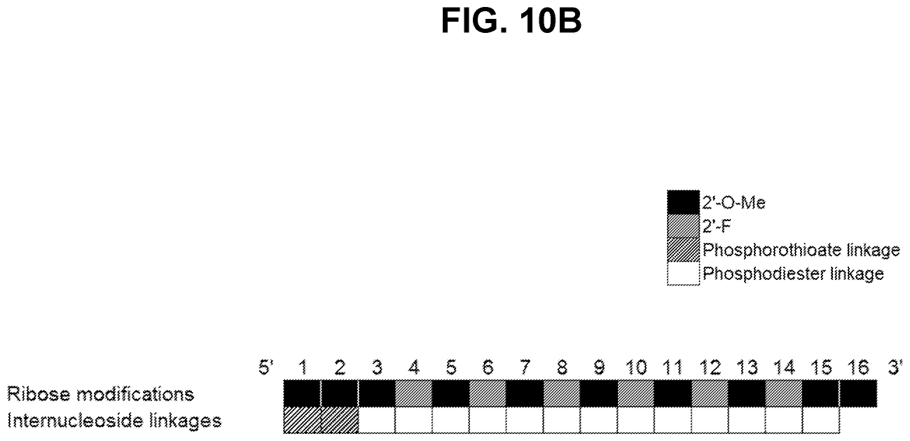

A-S-A-S-A-O-B-O-A-O-B-O-A-O-B-O-A-O-B-O-A-O-B-O-A-O-B-O-A-O-A (FIG. 10B) Sense Pattern 6 (Formula S6):

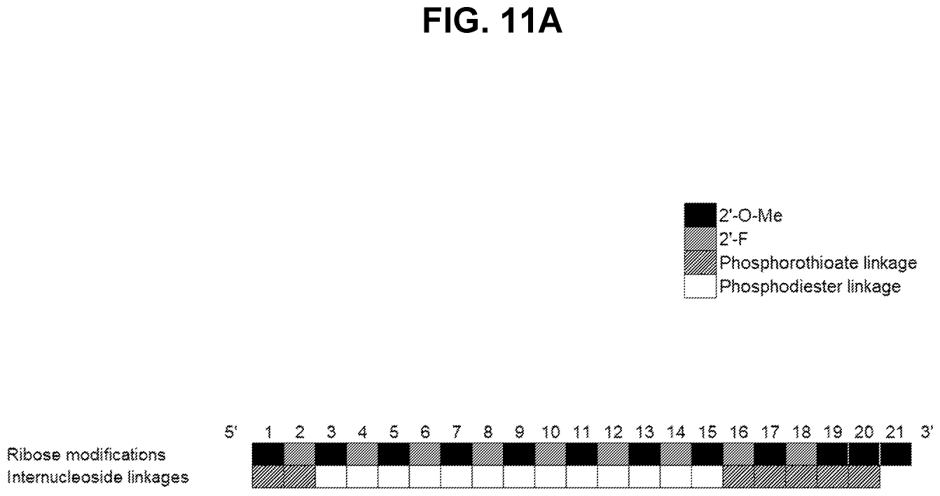

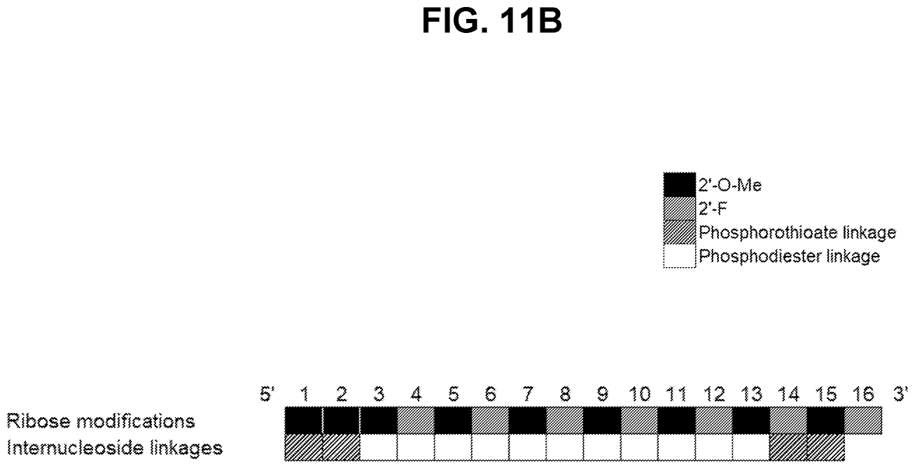

A-S-A-S-A-O-B-O-A-O-B-O-A-O-B-O-A-O-B-O-A-O-B-O-A-O-B-S-A-S-B (FIG. 11B); Sense Pattern 7 (Formula S7):

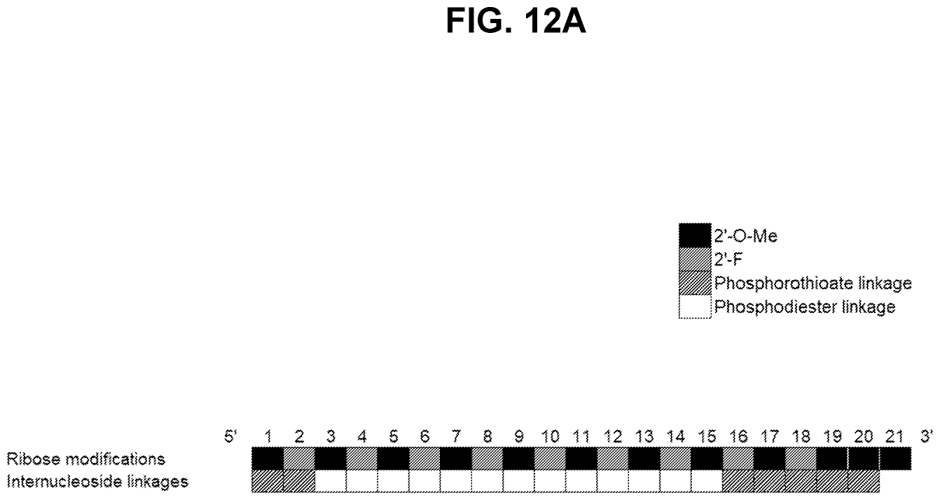

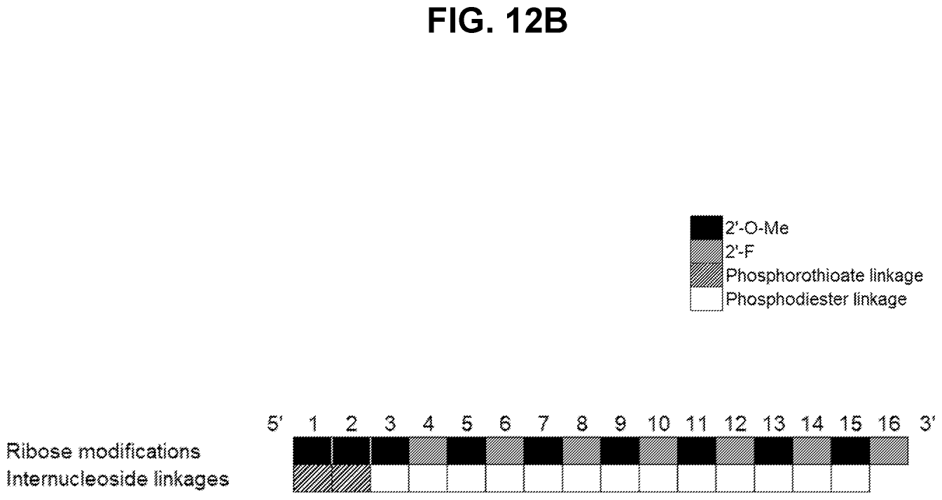

A-S-A-S-A-O-B-O-A-O-B-O-A-O-B-O-A-O-B-O-A-O-B-O-A-O-B-O-A-O-B (FIG. 12B) Sense Pattern 8 (Formula S8):

wherein A represents a 2′-O-Me ribonucleoside, B represents a 2′-F ribonucleoside, O represents a phosphodiester internucleoside linkage, and S represents a phosphorothioate internucleoside linkage.

The antisense and sense strand of the ds-siRNA molecule are each produced such that the resulting duplex structure has 0 to 5 nucleotide mismatches (e.g., 0, 1, 2, 3, 4, or 5) mismatches between the sense strand and the antisense strand and/or the antisense strand and the target mRNA sequence. The ds-siRNA molecule may be further modified to incorporate a 5′ phosphorus stabilizing moiety (e.g., a 5′-vinylphosphonate) and/or a hydrophobic moiety (e.g., cholesterol, vitamin D, or tocopherol) on the antisense strand, sense strand, or both. In addition, the ds-siRNA molecule may contain branched structures disclosed herein, such as di-branched, tri-branched, or tetra-branched structures disclosed herein. The ds-siRNA agent may be further incorporated into a pharmaceutical composition containing a pharmaceutically acceptable excipient, carrier, or diluent.

### Example 3: Method of Preparing a ds-siRNA Molecule Having Patterned Ribonucleoside Modifications and Internucleoside Linkage Modifications (III)

A ds-siRNA molecule having patterned ribonucleoside modifications and internucleoside linkage modifications of the disclosure is prepared according to methods well-known in the art, such as methods disclosed herein. The ds-siRNA molecule is a duplex oligoribonucleotide in which the sense strand is derived from and has full (i.e., 100%) or partial (e.g., at least 70%, 75%, 80%, 85%, 90%, 95%, 96%, 97%, 98%, 99%, or more) sequence identity to an mRNA sequence of a target gene. The nucleic acid sequence of the antisense strand is fully (i.e., 100%) complementary or partially (e.g., at least 70%, 75%, 80%, 85%, 90%, 95%, 96%, 97%, 98%, 99%, or more) complementary to the nucleic acid sequence of the sense strand/mRNA of target gene. The antisense and sense strands of the ds-siRNA agent are each synthesized according to well-known methods (e.g., synthesis and ligation or tandem synthesis) to contain alternating patterns (i.e., motifs) of modified ribonucleosides, such as 2′-O-Me and 2′-F ribonucleosides and modified internucleoside linkages, such as phosphorothioate linkages. The antisense strand is produced to be of a desirable length such that a functional benefit (e.g., RNA interference, thermal stability, and/or resistance against nucleases) is achieved. An exemplary antisense strand may be, e.g., 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. The sense strand is produced to be of a desirable length such that a functional benefit (e.g., efficient RISC loading, thermal stability, and/or resistance against nucleases) is achieved. An exemplary sense strand may be, e.g., 15 nucleotides,16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. Consequent to the difference in length between the antisense strand and the sense strand, the ds-siRNA duplex structure contains a 5′ overhang, 3′ overhang, or both. An exemplary antisense strand may have the following pattern:

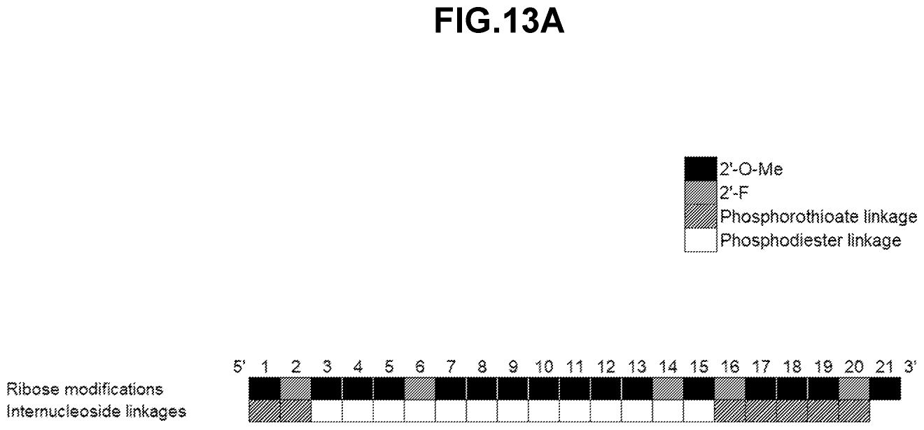

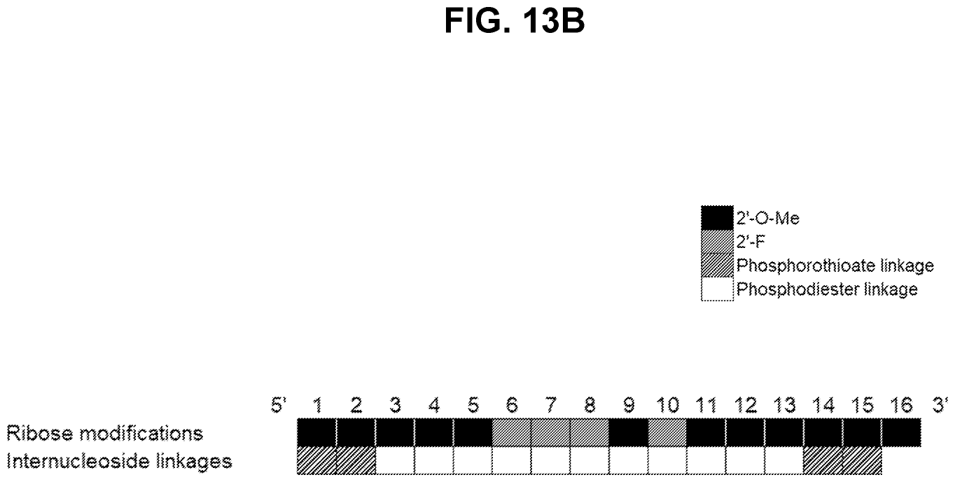

A-S-B-S-A-O-A-O-A-O-B-O-A-O-A-O-A-O-A-O-A-O-A-O-A-O-B-O-A-O-B-S-A-S-A-S-A-S-B-S-A (FIG. 13A); Antisense Pattern 4 (Formula A4):

An exemplary sense strand may have the following pattern:

A-S-A-S-A-O-A-O-A-O-B-O-B-O-B-O-A-O-B-O-A-O-A-O-A-O-A-S-A-S-A (FIG. 13B) Sense Pattern 9 (Formula S9):

wherein A represents a 2′-O-Me ribonucleoside, B represents a 2′-F ribonucleoside, O represents a phosphodiester internucleoside linkage, and S represents a phosphorothioate internucleoside linkage. The antisense and sense strand of the ds-siRNA molecule are each produced such that the resulting duplex structure has 0 to 5 nucleotide mismatches (e.g., 0, 1, 2, 3, 4, or 5) mismatches between the sense strand and the antisense strand and/or the antisense strand and the target mRNA sequence. The ds-siRNA molecule may be further modified to incorporate a 5′ phosphorus stabilizing moiety (e.g., a 5′-vinylphosphonate) and/or a hydrophobic moiety (e.g., cholesterol, vitamin D, or tocopherol) on the antisense strand, sense strand, or both. In addition, the ds-siRNA molecule may contain branched structures disclosed herein, such as di-branched, tri-branched, or tetra-branched structures disclosed herein. The ds-siRNA agent may be further incorporated into a pharmaceutical composition containing a pharmaceutically acceptable excipient, carrier, or diluent.

### Example 4: Method of Delivering a ds-siRNA Molecule to the Central Nervous System of a Patient

A subject, such as a human subject, diagnosed with a disease is treated with a dose and frequency determined by a practitioner (e.g., three times daily, twice daily, once daily, once weekly, once monthly) by administering the siRNA molecule of the disclosure of a pharmaceutical composition containing the same. Dosage and frequency are determined based on the subject's height, weight, age, sex, and other disorders.

A siRNA molecule (e.g., a branched siRNA molecule) having a pattern of chemical modifications disclosed herein is selected by the practitioner for compatibility with the disease and subject. Single- or double-stranded branched siRNA are available for selection. The siRNA chosen has an antisense strand, and in the case of double-stranded siRNA, a sense strand with a sequence and RNA modifications (e.g., natural and non-natural internucleoside linkages, modified sugars, and 5′-phosphorus stabilizing moieties) best suited to the patient and the disease being targeted. For example, the antisense strand may have any one of the antisense strand modification patterns disclosed herein, such as, e.g., Antisense Pattern 2: A-S-B-S-A-O-B-O-B-O-B-O-A-O-B-O-A-O-B-O-A-O-B-O-A-O-B-O-A-O-B-S-A-S-A-S-A-S-A-S-A (Formula A2); Antisense Pattern 3: A-S-B-S-A-O-B-O-A-O-B-O-A-O-B-O-A-O-B-O-A-O-B-O-A-O-B-O-A-O-B-S-A -S-B-S-A-S-A-S-A (Formula A3); or Antisense Pattern 4: A-S-B-S-A-O-A-O-A-O-B-O-A-O-A-O-A-O-A-O -A-O-A-O-A-O-B-O-A-O-B-S-A-S-A-S-A-S-B-S-A (Formula A4). In the case of a ds-siRNA, Antisense Pattern 1 may have a fully or partially complementary sense strand having any one of the patterns of chemical modifications of Sense Pattern 1: A-S-A-S-A-O-B-O-A-O-B-O-A-O-B-O-A-O-B-O-A-O-A-O-A-O -B-S-A-S-A (Formula S1); Sense Pattern 2: A-S-A-S-A-O-B-O-A-O-B-O-A-O-B-O-A-O-B-O-A-O-A-O-A-O -B-O-A-O-A (Formula S2); Sense Pattern 3: A-S-A-S-A-O-B-O-A-O-B-O-A-O-B-O-A-O-B-O-A-O-A-O-A-O -B-S-A-S-B (Formula S3); or Sense Pattern 4: A-S-A-S-A-O-B-O-A-O-B-O-A-O-B-O-A-O-B-O-A-O-A-O-A -O-B-O-A-O-B (Formula S4). In the case of a ds-siRNA having an Antisense Pattern 2, the sense strand may have any one of the patterns of chemical modifications of Sense Pattern 5: A-S-A-S-A-O-B-O-A-O-B -O-A-O-B-O-A-O-B-O-A-O-B-O-A-O-B-S-A-S-A (Formula S5); Sense Pattern 6: A-S-A-S-A-O-B-O-A-O-B -O-A-O-B-O-A-O-B-O-A-O-B-O-A-O-B-O-A-O-A (Formula S6); Sense Pattern 7: A-S-A-S-A-O-B-O-A-O-B -O-A-O-B-O-A-O-B-O-A-O-B-O-A-O-B-S-A-S-B (Formula S7); or Sense Pattern 8: A-S-A-S-A-O-B-O-A-O -B-O-A-O-B-O-A-O-B-O-A-O-B-O-A-O-B-O-A-O-B (Formula S8). In the case of a ds-siRNA having an Antisense Pattern 3, the sense strand may have a sense strand having a pattern of modifications of Sense Pattern 9: A-S-A-S-A-O-A-O-A-O-B-O-B-O-B-O-A-O-B-O-A-O-A-O-A-O-A-S-A-S-A (Formula S9); wherein A and B are different nucleosides (e.g., A is a 2-O-methyl ribonucleoside; B is a 2′-fluoro ribonucleoside), T is phosphorothioate, P is a phosphodiester, and PSM is a 5′-phosphorus stabilizing moiety (e.g., 5′-vinylphosphonate).

The siRNA is delivered by the route best suited the patient and condition (e.g., intrathecally, intracerebroventricularly, or intrastriatally), at a rate tolerable to the patient until the subject has reached a maximum tolerated dose, or until the symptoms of the disease are ameliorated satisfactorily.

### Example 5: In Vivo Gene Silencing Using di-siRNA Pattern Variants in a Murine Model

To determine the efficacy of gene silencing using the di-siRNA constructs described herein, gene silencing was performed on the huntingtin (HTT) gene using a ds-siRNA pattern variant in FVB/NJ female mice in vivo.

### Methods

In vivo administration of di-siRNA. 13 Di-siRNA scaffolds with a common sequence targeting HTT were designed and tested in vivo in FVB/NJ female mice with intracerebroventricular (ICV) dosing. Of these ds-siRNA scaffolds, four scaffolds, namely ds-siRNA_A_V1, ds-siRNA_A_V2, ds-siRNA_B_V1, and ds-siRNA_B_V2 were first generation (F1) ds-siRNA molecules used to compare knockdown efficacy with respect to the remaining second generation (F2) ds-siRNA scaffolds. Animals were divided into 14 groups (13 Di-siRNA treatment and 1 PBS vehicle) with 10 animals per group and intracerebroventricularly injected with the test articles shown in Table 2. On day one, stereotactic injection was performed on FVB/NJ female mice, wherein single unilateral ICV injections (10 μL) were performed on the right side of brain at 0.5 μL/min after needle placement at the following coordinates from bregma: −0.45 mm AP, +1 mm mediolateral and −2.5 mm dorsoventral. Three dose levels were tested for each scaffold (0.1, 0.5, and 2.5 nmol total compound). At one month after injection, animals were perfused with cold 1× PBS and brains were collected and sliced. Tissue punches of fixed diameter and thickness were collected from different brain regions (motor cortex, hippocampus, and striatum) and snap frozen on dry ice.

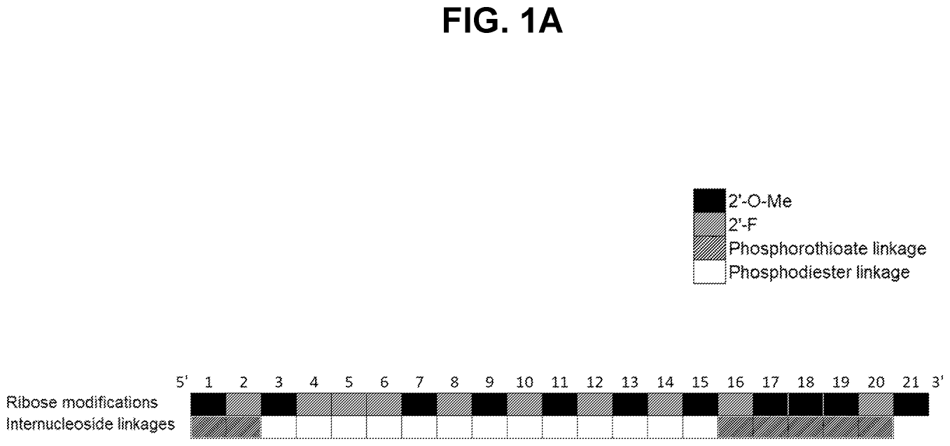

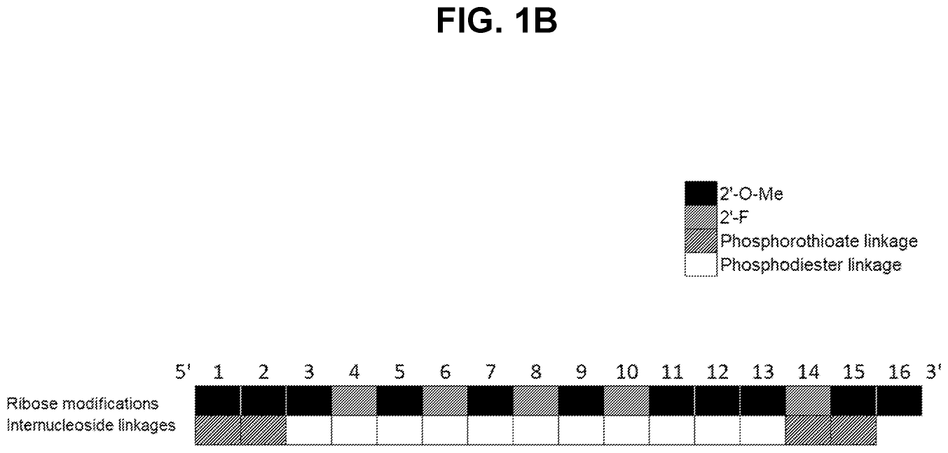

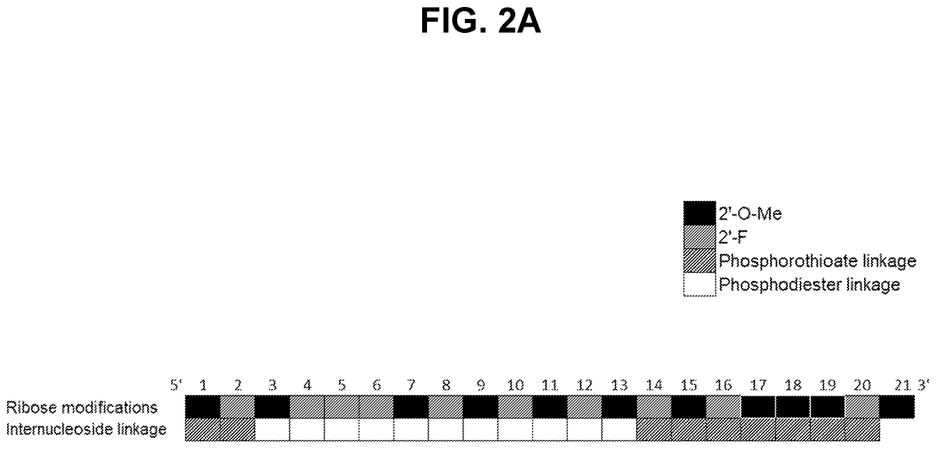

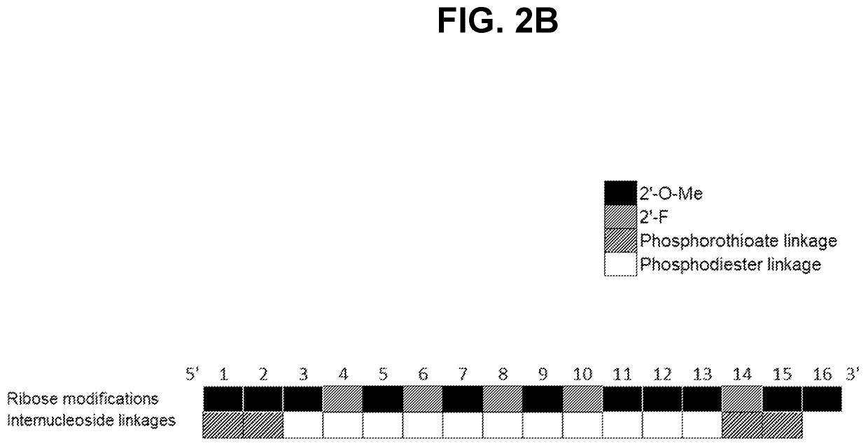

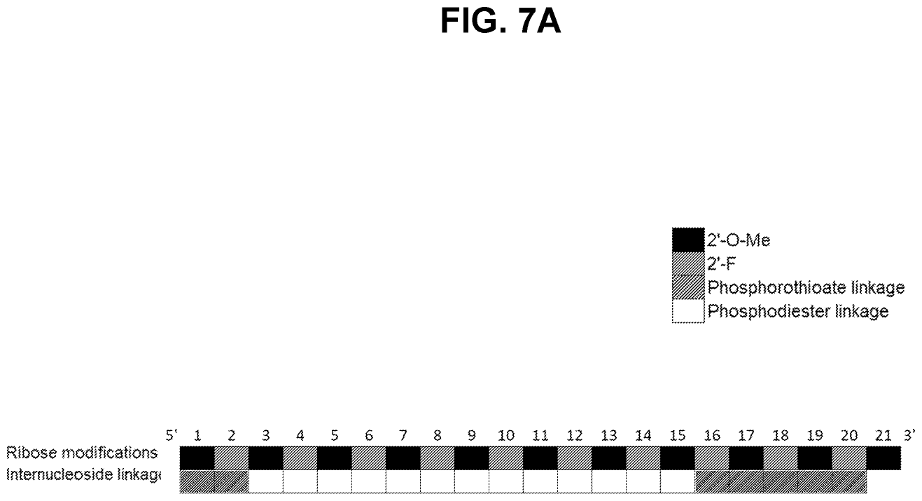

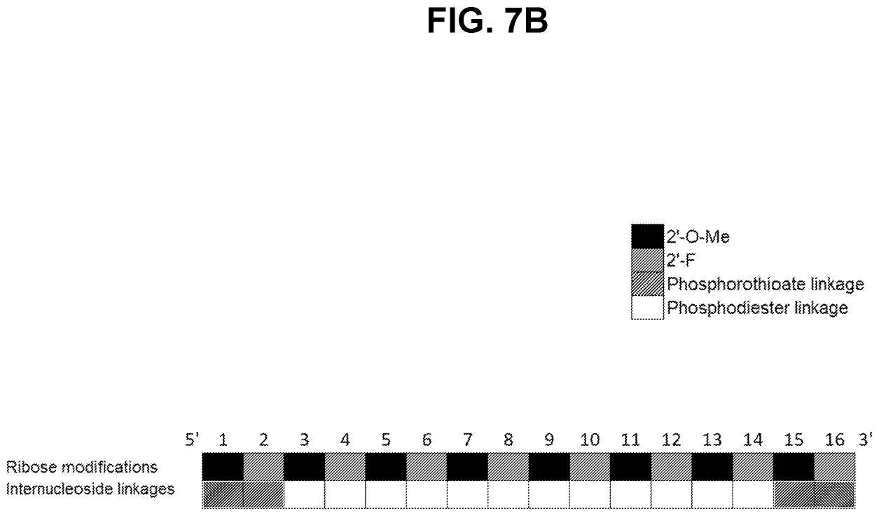

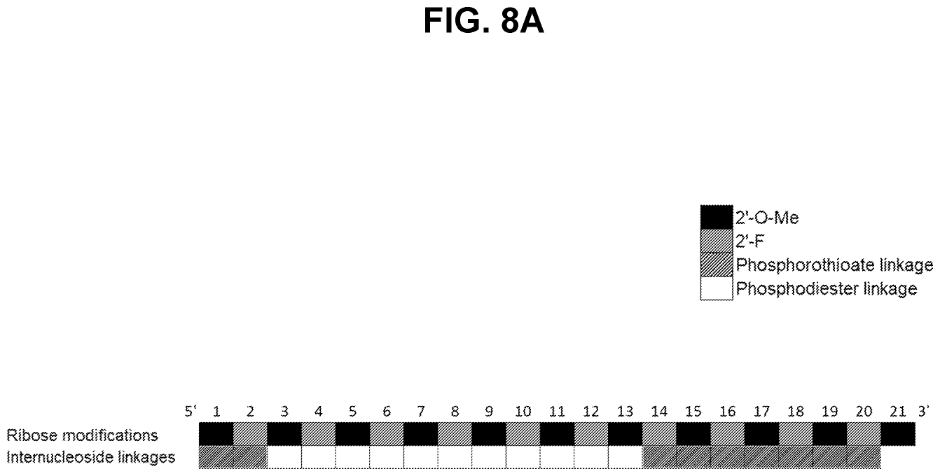

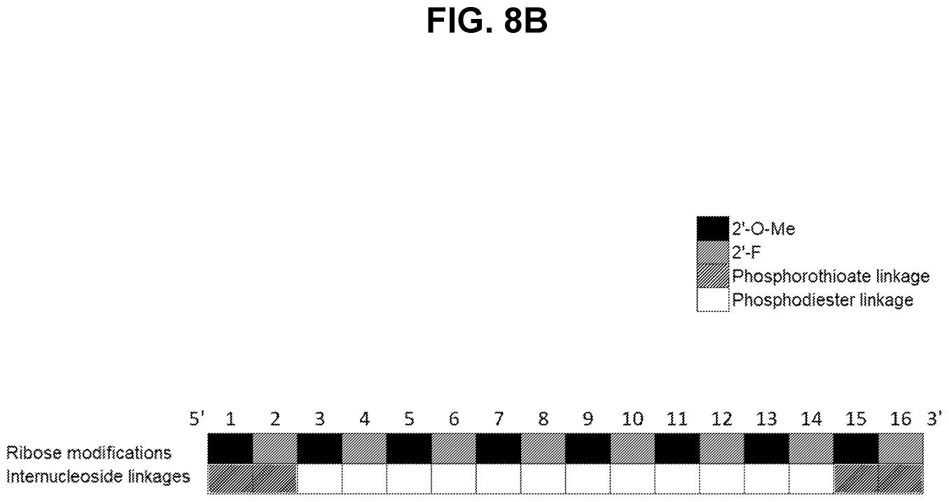

TABLE 2
Treatment groups

| | Schematic | |
|---|---|---|
| siRNA variants | (A = antisense; B = sense) | Generation |
| PBS | N/A | N/A |
| Ds-siRNA A_V1 | FIG. 1A & 1B | F1 |
| Ds-siRNA A_V2 | FIG. 2A & 2B | F1 |
| Ds-siRNA A_V3 | FIG. 3A & 3B | F2 |
| Ds-siRNA A_V4 | FIG. 4A & 4B | F2 |
| Ds-siRNA A_V5 | FIG. 5A & 5B | F2 |
| Ds-siRNA A_V6 | FIG. 6A & 6B | F2 |
| Ds-siRNA B_V1 | FIG. 7A & 7B | F1 |
| Ds-siRNA B_V2 | FIG. 8A & 8B | F1 |
| Ds-siRNA B_V3 | FIG. 9A & 9B | F2 |
| Ds-siRNA B_V4 | FIG. 10A & 10B | F2 |
| Ds-siRNA B_V5 | FIG. 11A & 11B | F2 |
| Ds-siRNA B_V6 | FIG. 12A & 12B | F2 |
| Ds-siRNA C_V1 | FIG. 13A & 13B | F2 |

RNA expression analysis. To evaluate Huntingtin mRNA expression levels, total RNA was extracted from mouse brain tissue punches using phenol:chloroform extraction. This method was performed by first disrupting tissue samples in TRIzol reagent (Invitrogen) using a TissueLyser II (Qiagen) and adding chloroform to the homogenized samples at a ratio of 5:1 TRIzol:chloroform. Tubes were then shaken vigorously and spun at 12,000×g for 15 mins and the resulting upper aqueous phase containing total RNA was carefully removed and added to a clean tube. An equal volume of 70% ethanol was added to each sample and mixed gently. The sample was further purified using Qiagen RNeasy column purification according to standard kit protocol. Samples were eluted in 40 μL RNase-free water. Following elution, RNA was analyzed on a TapeStation 4200 Bioanalyzer (Agilent) to assess concentration and quality. All samples were normalized for total RNA. cDNA synthesis was performed in a 20 μL reaction volume using the Applied Biosystems High-Capacity cDNA Reverse Transcription kit. Following RT-PCR, nuclease-free water was added to cDNA for a final sample volume of 50 μL.

For HTT gene expression analysis, qPCR was performed using TaqMan reagents on a QuantStudio7 Real-Time PCR instrument (Life Technologies). Samples were probed for mouse total HTT expression (assay ID Mm01213820_m1) and were normalized against in-well housekeeping gene reference ATP5b (Assay ID Mm00443967_g1). Comparative analysis was performed for relative HTT quantitation (DeltaDeltaCt) against samples taken from PBS treated mice. Results are showing HTT expression following administration of different doses of Di-siRNA are shown in Tables 3-5. Mean %=mean % HTT mRNA expression; SD=standard deviation; n=number of animals per group.

TABLE 3
Dose-dependent Htt mRNA knockdown by Di-siRNA
in different brain regions at 0.1 nmol dosing

| | 0.1 nmol dosing | | | | | | | | |
|---|---|---|---|---|---|---|---|---|---|
| | Hippocampus | | | Motor Cortex | | | Striatum | | |
| Construct tested | Mean % | SD | n | Mean % | SD | n | Mean % | SD | n |
| Ds-siRNA B_V1 | 56.4 | 12.2 | 10 | 73.0 | 12.0 | 10 | 84.4 | 9.8 | 7 |
| Ds-siRNA B_V2 | 52.1 | 9.6 | 9 | 77.3 | 8.4 | 10 | 80.6 | 8.7 | 10 |
| Ds-siRNA B_V3 | 57.1 | 12.3 | 9 | 80.1 | 19.5 | 10 | 93.3 | 24.7 | 9 |
| Ds-siRNA B_V4 | 54.3 | 15.6 | 9 | 51.2 | 8.2 | 9 | 65.4 | 14.8 | 9 |
| Ds-siRNA B_V5 | 44.8 | 7.1 | 10 | 53.8 | 9.6 | 10 | 71.7 | 9.4 | 10 |
| Ds-siRNA B_V6 | 66.6 | 28.2 | 9 | 65.3 | 23.7 | 9 | 69.4 | 15.8 | 9 |
| Ds-siRNA A_V1 | 45.6 | 8.5 | 9 | 60.8 | 21.3 | 10 | 74.8 | 16.9 | 10 |
| Ds-siRNA A_V2 | 57.1 | 16.1 | 10 | 55.8 | 16.8 | 10 | 66.3 | 13.5 | 10 |
| Ds-siRNA A_V3 | 54.5 | 17.3 | 10 | 54.0 | 14.3 | 10 | 67.3 | 11.1 | 10 |
| Ds-siRNA A_V4 | 39.2 | 16.2 | 10 | 48.7 | 16.3 | 10 | 65.0 | 10.7 | 10 |
| Ds-siRNA A_V5 | 52.3 | 14.9 | 10 | 58.1 | 13.2 | 10 | 67.7 | 19.1 | 10 |
| Ds-siRNA A_V6 | 38.2 | 6.1 | 9 | 54.9 | 15.6 | 9 | 68.4 | 9.2 | 9 |
| Ds-siRNA C_V1 | 41.9 | 7.7 | 9 | 51.0 | 6.0 | 9 | 57.5 | 6.8 | 9 |

TABLE 4
Dose-dependent Htt mRNA knockdown by Di-siRNA
in different brain regions at 0.5 nmol dosing

| | 0.5 nmol dosing | | | | | | | | |
|---|---|---|---|---|---|---|---|---|---|
| | Hippocampus | | | Motor Cortex | | | Striatum | | |
| Construct tested | Mean % | SD | n | Mean % | SD | n | Mean % | SD | n |
| Ds-siRNA B_V1 | 44.8 | 12.3 | 10 | 47.3 | 11.4 | 10 | 60.1 | 9.7 | 7 |
| Ds-siRNA B_V2 | 35.9 | 5.7 | 10 | 46.8 | 14.1 | 10 | 57.3 | 17.2 | 10 |
| Ds-siRNA B_V3 | 42.9 | 9.8 | 10 | 40.4 | 9.3 | 10 | 54.6 | 11.9 | 10 |
| Ds-siRNA B_V4 | 32.8 | 6.3 | 10 | 31.0 | 5.0 | 10 | 35.5 | 7.4 | 10 |
| Ds-siRNA B_V5 | 33.1 | 6.7 | 10 | 42.2 | 11.9 | 10 | 61.4 | 15.7 | 10 |
| Ds-siRNA B_V6 | 30.8 | 5.1 | 10 | 28.7 | 3.1 | 10 | 38.7 | 6.9 | 10 |
| Ds-siRNA A_V1 | 33.9 | 6.8 | 10 | 41.9 | 19.9 | 10 | 54.9 | 13.3 | 10 |
| Ds-siRNA A_V2 | 40.5 | 11.2 | 10 | 34.8 | 8.1 | 10 | 47.3 | 9.7 | 10 |
| Ds-siRNA A_V3 | 31.0 | 3.2 | 10 | 34.6 | 5.7 | 10 | 49.0 | 9.5 | 10 |
| Ds-siRNA A_V4 | 27.0 | 3.1 | 10 | 35.5 | 7.6 | 10 | 44.7 | 11.0 | 10 |
| Ds-siRNA A_V5 | 46.0 | 13.4 | 10 | 50.5 | 22.0 | 9 | 52.0 | 9.8 | 10 |
| Ds-siRNA A_V6 | 32.4 | 6.3 | 10 | 35.8 | 9.9 | 10 | 46.2 | 9.7 | 10 |
| Ds-siRNA C_V1 | 30.9 | 5.3 | 10 | 40.1 | 10.5 | 10 | 46.3 | 8.1 | 10 |

TABLE 5
Dose-dependent Htt mRNA knockdown by Di-siRNA
in different brain regions at 2.5 nmol dosing

| | 2.5 nmol dosing | | | | | | | | |
|---|---|---|---|---|---|---|---|---|---|
| | Hippocampus | | | Motor Cortex | | | Striatum | | |
| Construct tested | Mean % | SD | n | Mean % | SD | n | Mean % | SD | n |
| Ds-siRNA B_V1 | 34.7 | 3.9 | 10 | 28.9 | 3.0 | 10 | 41.5 | 20.6 | 9 |
| Ds-siRNA B_V2 | 32.7 | 3.6 | 10 | 27.0 | 3.7 | 10 | 34.3 | 4.6 | 10 |
| Ds-siRNA B_V3 | 30.2 | 3.0 | 10 | 29.4 | 4.5 | 10 | 42.3 | 7.7 | 10 |
| Ds-siRNA B_V4 | 28.9 | 10.6 | 10 | 27.8 | 14.7 | 10 | 28.3 | 7.4 | 10 |
| Ds-siRNA B_V5 | 31.3 | 3.2 | 10 | 26.6 | 5.5 | 10 | 37.6 | 7.6 | 10 |
| Ds-siRNA B_V6 | 29.7 | 4.3 | 10 | 27.2 | 4.6 | 9 | 32.0 | 8.1 | 10 |
| Ds-siRNA A_V1 | 31.0 | 7.9 | 10 | 36.1 | 18.7 | 10 | 39.4 | 11.7 | 10 |
| Ds-siRNA A_V2 | 30.6 | 3.7 | 10 | 26.6 | 4.0 | 10 | 32.2 | 2.7 | 10 |
| Ds-siRNA A_V3 | 26.9 | 3.2 | 10 | 23.5 | 4.5 | 10 | 29.8 | 5.9 | 10 |
| Ds-siRNA A_V4 | 26.9 | 6.0 | 10 | 27.8 | 5.3 | 10 | 34.8 | 10.3 | 10 |
| Ds-siRNA A_V5 | 30.4 | 4.0 | 10 | 28.2 | 4.3 | 10 | 31.6 | 4.1 | 10 |
| Ds-siRNA A_V6 | 26.6 | 4.9 | 9 | 25.0 | 2.6 | 9 | 31.8 | 3.1 | 9 |
| Ds-siRNA C_V1 | 33.3 | 11.5 | 10 | 32.1 | 9.6 | 10 | 31.8 | 11.6 | 9 |

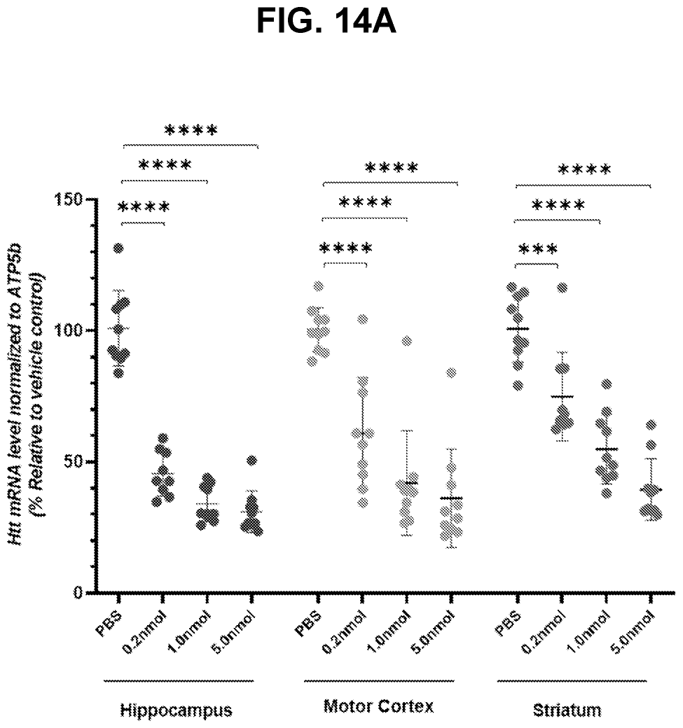

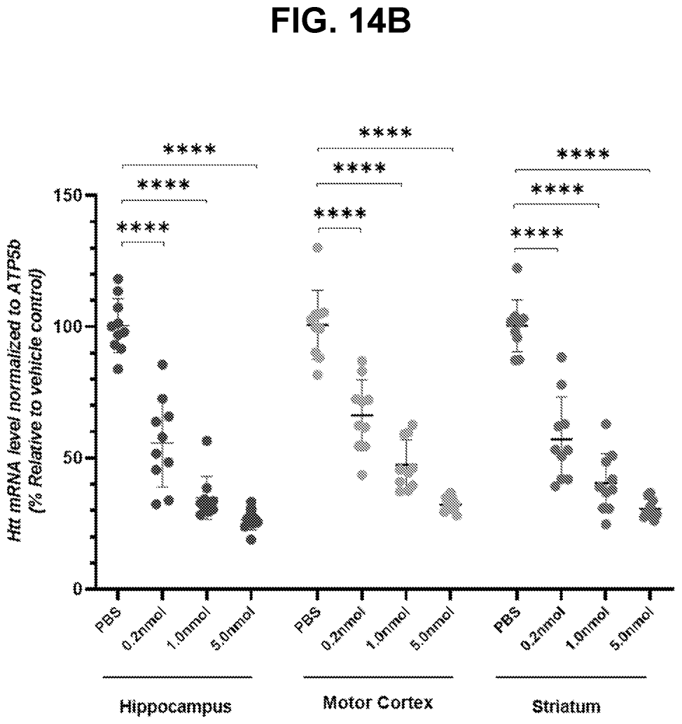

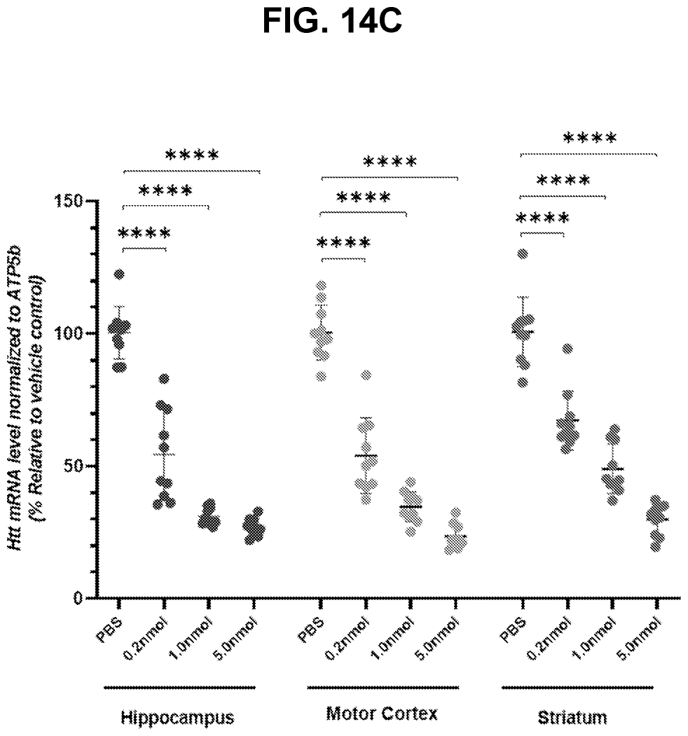

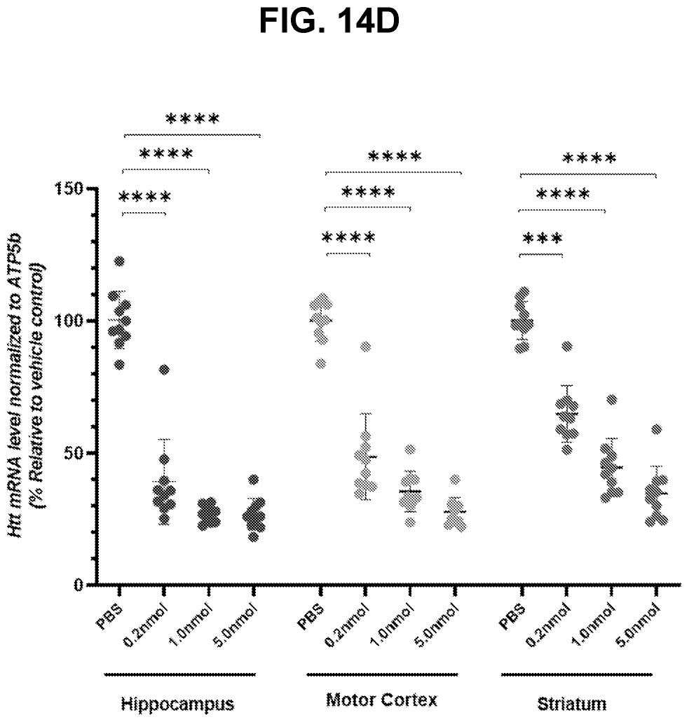

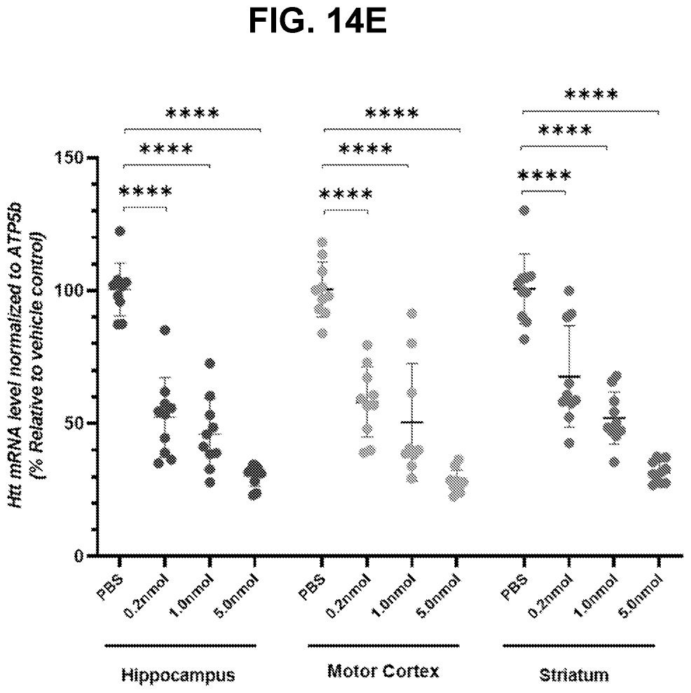

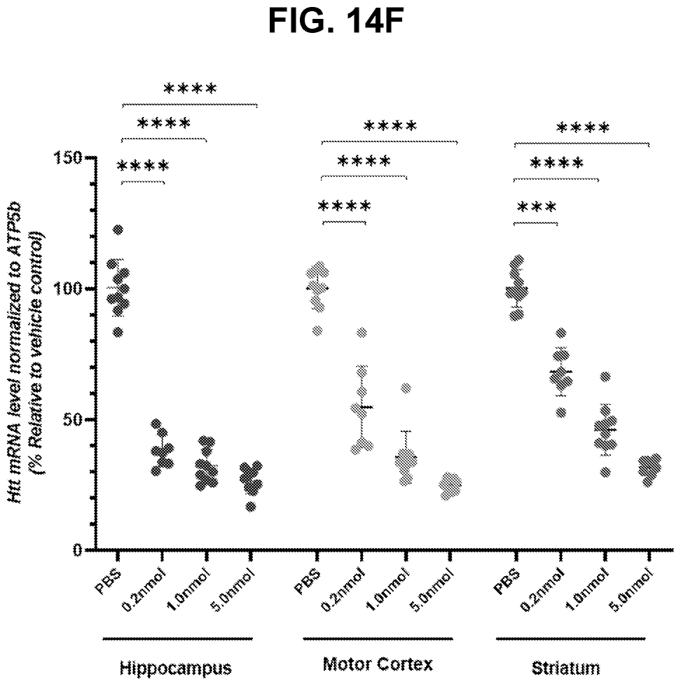

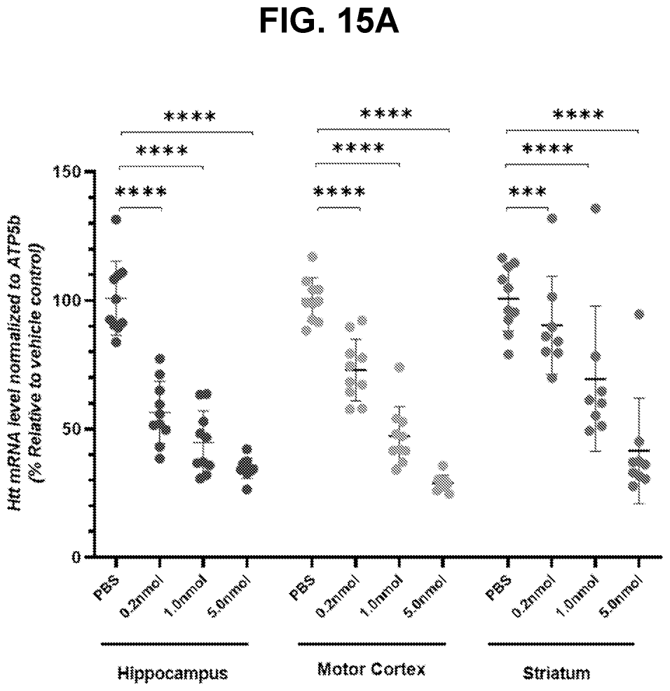

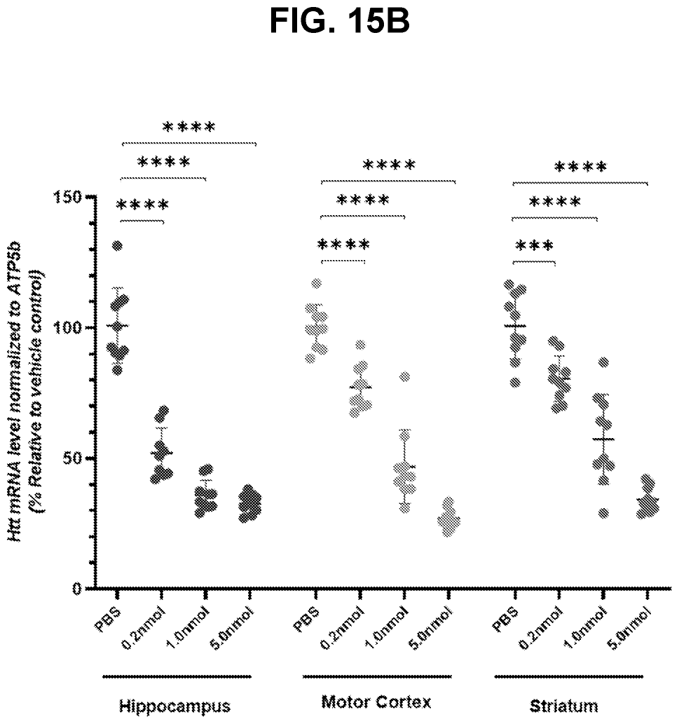

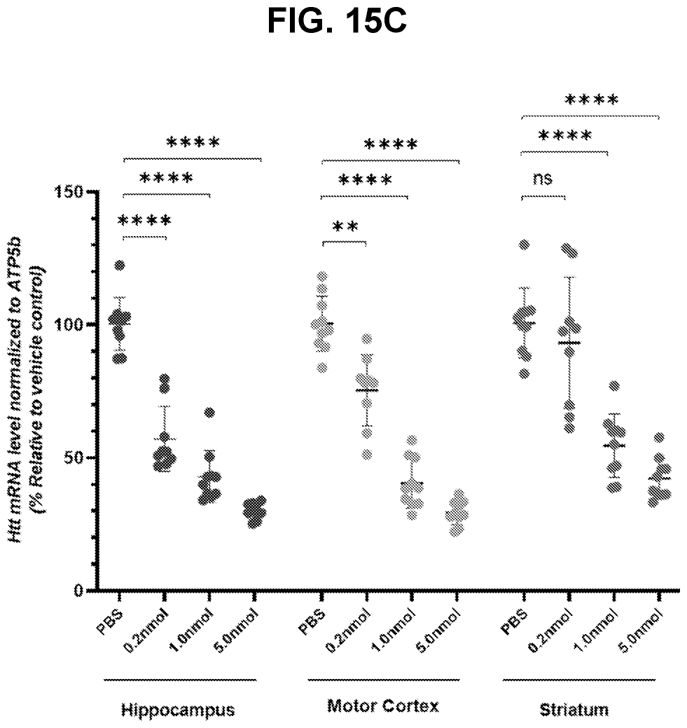

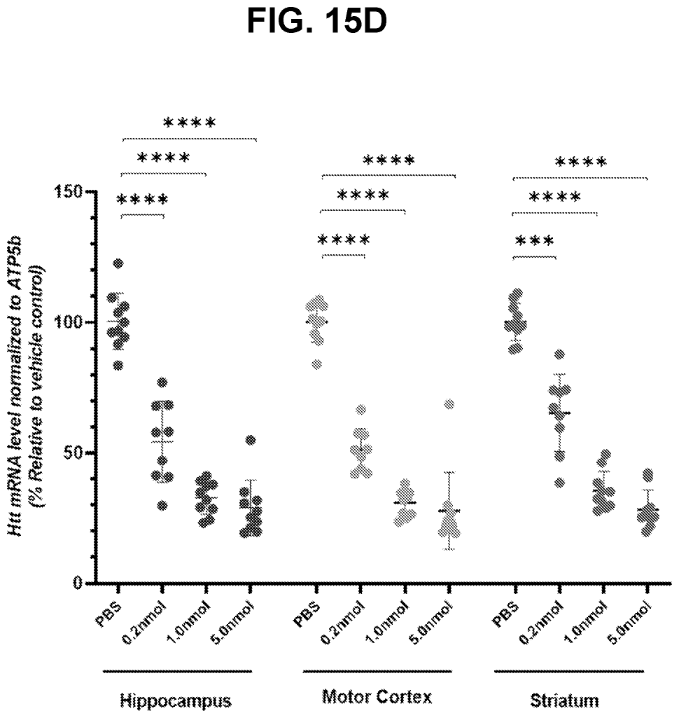

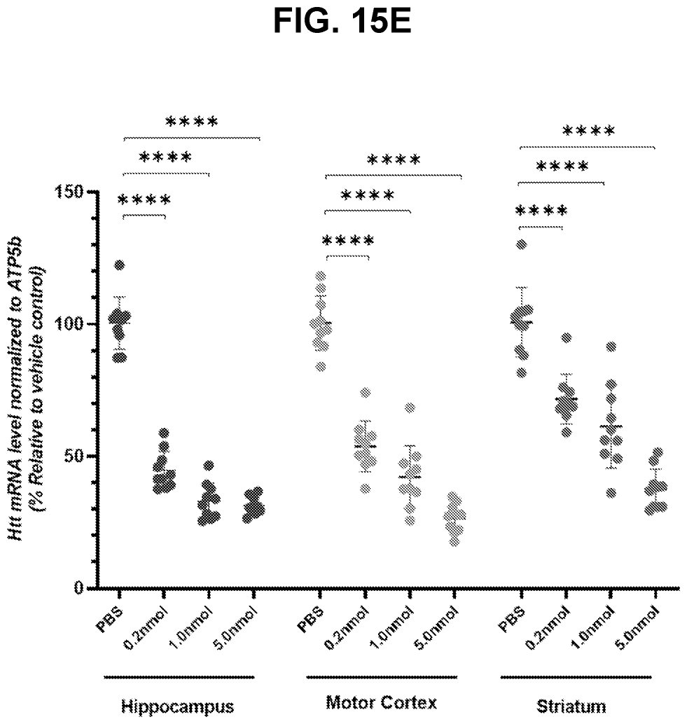

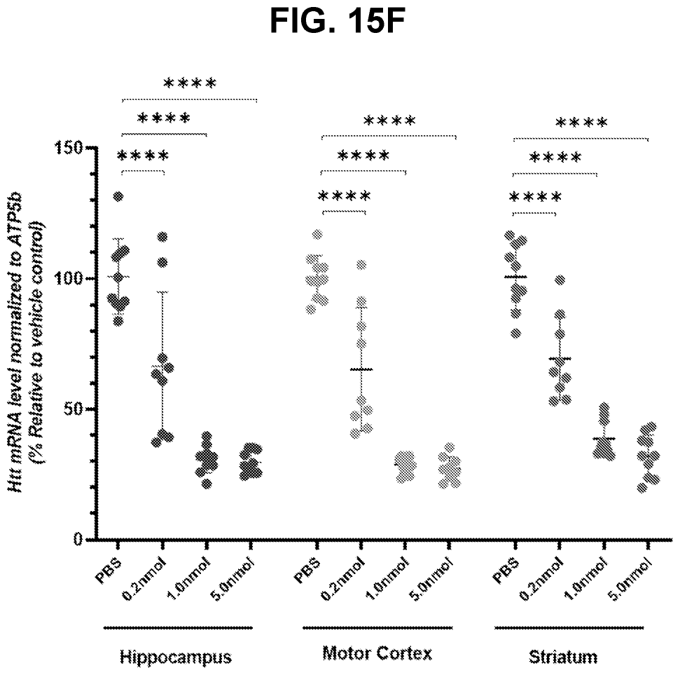

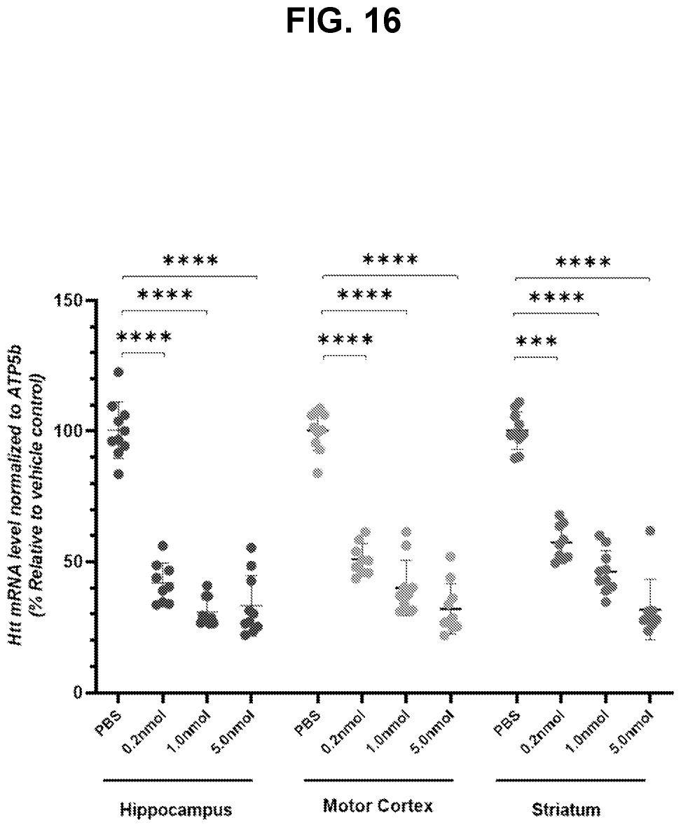

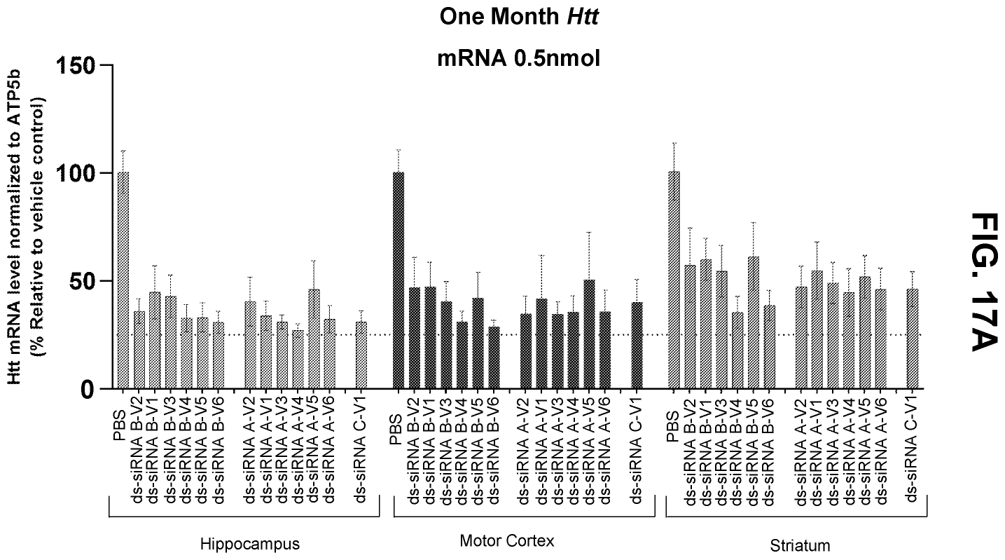

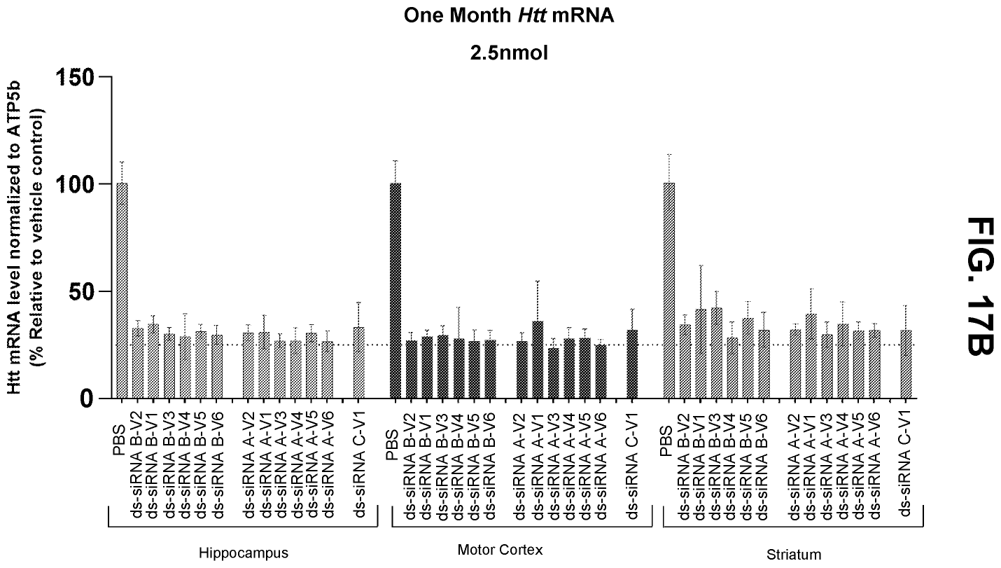

Each of the tested ds-siRNA constructs tested produced a dose-dependent reduction in HTT mRNA levels in each of the tested brain regions of the HTT mRNA levels following knockdown. Exemplary scatter plots demonstrating the dose-dependence of HTT knockdown in mice using the ds-siRNA constructs of the disclosure are provided for each of the ds-siRNA A, ds-siRNA B, and ds-siRNA C constructs in FIGS. 14A-14F, FIGS. 15A-15F, and FIG. 16, respectively.

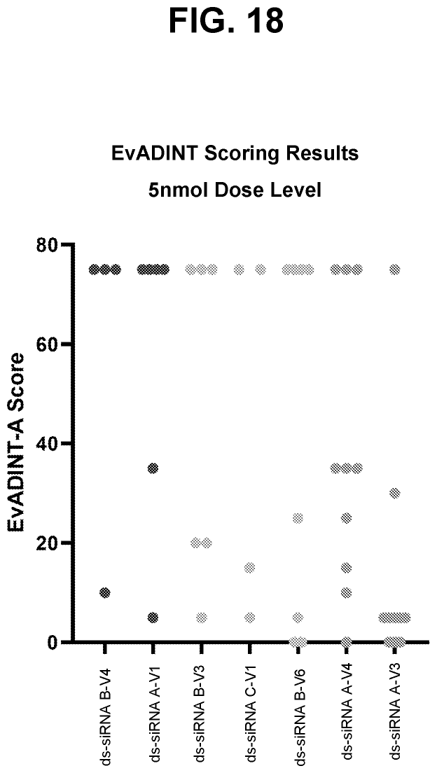

Additional testing was carried out to compare the reduction in HTT mRNA levels of multiple patterns in relation to each other at 0.5 nmol and 2.5 nmol dose levels as shown in FIG. 18A and FIG. 18B, respectively. mRNA levels were tested after one month. FIG. 18B shows several of the F2 patterns have increased potency over the comparative F1 patterns.

### Example 6: Mitigating Toxic Effects of Interfering RNA Delivery to the Central Nervous System

### Introduction

In many species, introduction of double-stranded RNA induces potent and specific gene silencing by way of RNA interference (RNAi). This phenomenon occurs in both plants and animals and has roles in viral defense and transposon silencing mechanisms. For example, short interfering RNAs (siRNAs), which are generally much shorter than the target gene, have been shown to be effective at gene silencing and are, therefore, useful as therapeutic agents for silencing genes to restore genetic and biochemical pathway activity from a disease state towards a normal, healthy state. However, delivery of interfering RNA molecules, such as short interfering RNA (siRNA), to a subject, particularly to the subject's central nervous system, carries the risk of toxic side effects, including seizures, tremors, and hyperactive motor behaviors, among others. There remains a need for interfering RNA molecules that effectuate reduced toxicity upon administration to a subject in need thereof.

### Results

The severity of acute CNS toxicity of siRNA molecules of the disclosure was quantified by using an EvADINT Scoring Assay (Table 6). The higher the score, the more toxic the experimental condition was considered.

TABLE 6
EvADINT Scoring Assay

| Behavioral Element | EvADINT Scoring Assay | | | |
|---|---|---|---|---|
| Death | 75 | | | If no major/severe acute tox |
| Severity | Mild | Moderate | Severe | effects are observed in the |
| Seizure/Tremor | 10 | 15 | 20 | first 2 hours, monitoring of |
| Hyperactivity or other motor | 5 | 10 | 15 | recovery behaviors can be |
| behaviors | | | | stopped at this point. |
| | | | | Otherwise, monitoring animals |
| | | | | for behaviors described below |
| Time required for recovery (h) | 0.5 h | 1 h | 2 h | 24 h/no recovery |
| Sternal posture | 0 | 5 | 10 | 20 |
| Unstimulated movement | 0 | 5 | 10 | 15 |
| Movement without ataxia | | | | |

Various motifs of an exemplary siRNA molecule of the disclosure were evaluated for their toxicity benefit using the EvADINT scoring assay, with the average over multiple trials shown in Table 7, below. The data are graphically represented as a scatter plot in FIG. 19. As shown in the data, several F2 molecules (e.g., ds-siRNA A_V3, ds-siRNA A_V4, ds-siRNA B_V6, ds-siRNA C_V2, and ds-siRNA B_V3) show an improved toxicity benefit when compared to an F1 molecule (e.g., ds-siRNA A_V1)

TABLE 7
Toxicity evaluation of an exemplary
siRNA molecule of the disclosure

| Motif | Average EvADINT |
|---|---|
| ds-siRNA A_V3 | 20 |
| ds-siRNA A_V4 | 28.33 |
| ds-siRNA B_V6 | 30 |
| ds-siRNA C_V2 | 42.5 |
| ds-siRNA B_V3 | 45 |
| ds-siRNA A_V1 | 56.67 |
| ds-siRNA B_V4 | 58.75 |

### Specific Embodiments

Some specific embodiments are listed below. The below enumerated embodiments should not be construed to limit the scope of the disclosure, rather, the below are presented as some examples of the utility of the disclosure.

E1. A small interfering RNA (siRNA) molecule including an antisense strand and a sense strand having complementarity to the antisense strand, wherein the antisense strand includes a structure represented by Formula I, wherein Formula I is, in the 5′-to-3′ direction:

A-B-(A′)ⱼ-C-P²-D-P¹-(C′-P¹)ₖ-C′ Formula I;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′-O-methyl (2′-O-Me) ribonucleoside;
each C′, independently, is a 2′-O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.
   E2. The siRNA molecule of E1, wherein the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.
   E3. A small interfering RNA (siRNA) molecule including an antisense strand and a sense strand having complementarity to the antisense strand, wherein the antisense strand includes a structure represented by Formula II, wherein Formula II is, in the 5′-to-3′ direction:

A-B-(A′)ⱼ-C-P²-D-P¹-(C-P¹)ₖ-C′ Formula II;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′-O-methyl (2′-O-Me) ribonucleoside;
each C′, independently, is a 2′-O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.
   E4. The siRNA molecule of E1 or E3, wherein j is from 1 to 6.
   E5. The siRNA molecule of E1 or E3, wherein j is from 1 to 5.
   E6. The siRNA molecule of E1 or E3, wherein j is from 1 to 4.
   E7. The siRNA molecule of E1 or E3, wherein j is from 1 to 3.
   E8. The siRNA molecule of E1 or E3, wherein j is from 1 to 2.
   E9. The siRNA molecule of E1 or E3, wherein j is 1.
   E10. The siRNA molecule of E1 or E3, wherein j is 2.
   E11. The siRNA molecule of E1 or E3, wherein j is 3.
   E12. The siRNA molecule of E1 or E3, wherein j is 4.
   E13. The siRNA molecule of E1 or E3, wherein j is 5.
   E14. The siRNA molecule of E1 or E3, wherein j is 6.
   E15. The siRNA molecule of E1 or E3, wherein j is 7.
   E16. The siRNA molecule of any one of E1-E15, wherein k is from 1 to 6.
   E17. The siRNA molecule of E16, wherein k is from 1 to 5.
   E18. The siRNA molecule of E16, wherein k is from 1 to 4.
   E19. The siRNA molecule of E16, wherein k is from 1 to 3.
   E20. The siRNA molecule of E16, wherein k is from 1 to 2.
   E21. The siRNA molecule of E16, wherein k is 1.
   E22. The siRNA molecule of E16, wherein k is 2.
   E23. The siRNA molecule of E16, wherein k is 3.
   E24. The siRNA molecule of E16, wherein k is 4.
   E25. The siRNA molecule of E16, wherein k is 5.
   E26. The siRNA molecule of E16, wherein k is 6.
   E27. The siRNA molecule of E16, wherein k is 7.
   E28. The siRNA molecule of E1 or E3, wherein j is 4 and k is 4.
   E29. The siRNA molecule of E3, wherein the antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.
   E30. The siRNA molecule of any one of E1-E29, wherein the sense strand includes a structure represented by Formula III, wherein Formula III is, in the 5′-to-3′ direction:

E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7.
   E31. The siRNA molecule of E30, wherein m is from 1 to 6.
   E32. The siRNA molecule of E30, wherein m is from 1 to 5.
   E33. The siRNA molecule of E30, wherein m is from 1 to 4.
   E34. The siRNA molecule of E30, wherein m is from 1 to 3.
   E35. The siRNA molecule of E30, wherein m is from 1 to 2.
   E36. The siRNA molecule of E30, wherein m is 1.
   E37. The siRNA molecule of E30, wherein m is 2.
   E38. The siRNA molecule of E30, wherein m is 3.
   E39. The siRNA molecule of E30, wherein m is 4.
   E40. The siRNA molecule of E30, wherein m is 5.
   E41. The siRNA molecule of E30, wherein m is 6.
   E42. The siRNA molecule of E30, wherein m is 7.
   E43. The siRNA molecule of E30, wherein the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.
   E44. The siRNA molecule of E30, wherein the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.
   E45. The siRNA molecule of E30, wherein the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.
   E46. The siRNA molecule of E30, wherein the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.
   E47. An siRNA molecule including an antisense strand and a sense strand having complementarity to the antisense strand, wherein the antisense strand includes a structure represented by the formula IV, wherein Formula IV is, in the 5′-to-3′ direction:

A-(A′)ⱼ-C-P²-B-(C-P¹)ₖ-C′ Formula IV;
wherein A is represented by the formula C-P¹-D-P¹;
each A′, independently, is represented by the formula C-P²-D-P²;
B is represented by the formula D-P¹-C-P¹-D-P¹;
each C, independently, is a 2′-O-Me ribonucleoside;
each C′, independently, is a 2′-O-Me ribonucleoside or a 2′-F ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7
   E48. The siRNA molecule of E47, wherein j is from 1 to 6.
   E49. The siRNA molecule of E47, wherein j is from 1 to 5.
   E50. The siRNA molecule of E47, wherein j is from 1 to 4.
   E51. The siRNA molecule of E47, wherein j is from 1 to 3.
   E52. The siRNA molecule of E47, wherein j is from 1 to 2.
   E53. The siRNA molecule of any one of E47-E52, wherein j is 1.
   E54. The siRNA molecule of any one of E47-E52, wherein j is 2.
   E55. The siRNA molecule of any one of E47-E52, wherein j is 3.
   E56. The siRNA molecule of any one of E47-E52, wherein j is 4.
   E57. The siRNA molecule of any one of E47-E52, wherein j is 5.
   E58. The siRNA molecule of E47 or E48, wherein j is 6.
   E59. The siRNA molecule of E47, wherein j is 7.
   E60. The siRNA molecule of any one of E47-E59, wherein k is from 1 to 6.
   E61. The siRNA molecule of E60, wherein k is from 1 to 5.
   E62. The siRNA molecule of E60, wherein k is from 1 to 4.
   E63. The siRNA molecule of E60, wherein k is from 1 to 3.
   E64. The siRNA molecule of E60, wherein k is from 1 to 2.
   E65. The siRNA molecule of E60, wherein k is 1.
   E66. The siRNA molecule of E60, wherein k is 2.
   E67. The siRNA molecule of E60, wherein k is 3.
   E68. The siRNA molecule of E60, wherein k is 4.
   E69. The siRNA molecule of E60, wherein k is 5.
   E70. The siRNA molecule of E60, wherein k is 6.
   E71. The siRNA molecule of E60, wherein k is 7.
   E72. The siRNA molecule of E47, wherein j is 6 and k is 2.
   E73. The siRNA molecule of E47, wherein the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.
   E74. The siRNA molecule of any one of E47-E73, wherein the sense strand includes a structure represented by Formula V, wherein Formula V is, in the 5′-to-3′ direction:

E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹, and P² are as defined in Formula IV; and
m is an integer from 1 to 7.
   E75. The siRNA molecule of E74, wherein m is from 1 to 6.
   E76. The siRNA molecule of E74, wherein m is from 1 to 5.
   E77. The siRNA molecule of E74, wherein m is from 1 to 4.
   E78. The siRNA molecule of E74, wherein m is from 1 to 3.
   E79. The siRNA molecule of E74, wherein m is from 1 to 2.
   E80. The siRNA molecule of E74, wherein m is 1.
   E81. The siRNA molecule of E74, wherein m is 2.
   E82. The siRNA molecule of E74, wherein m is 3.
   E83. The siRNA molecule of E74, wherein m is 4.
   E84. The siRNA molecule of E74, wherein m is 5.
   E85. The siRNA molecule of E74, wherein m is 6.
   E86. The siRNA molecule of E74, wherein m is 7.
   E87. The siRNA molecule of E74, wherein the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.
   E88. The siRNA molecule of E74, wherein the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.
   E89. The siRNA molecule of E74, wherein the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.
   E90. The siRNA molecule of E74, wherein the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.
   E91. An siRNA molecule including an antisense strand and a sense strand having complementarity to the antisense strand, wherein the antisense strand includes a structure represented by Formula VI, wherein Formula VI is, in the 5′-to-3′ direction:

A-Bⱼ-E-Bₖ-E-F-Gₗ-D-P¹-C′ Formula VI;
wherein A is represented by the formula C-P¹-D-P¹;
each B, independently, is represented by the formula C-P²;
each C, independently, is a 2′-O-Me ribonucleoside;
each C′, independently, is a 2′-O-Me ribonucleoside or a 2′-F ribonucleoside;
each D, independently, is a 2′-F ribonucleoside;
each E, independently, is represented by the formula D-P²-C-P²;
F is represented by the formula D-P¹-C-P¹;
each G, independently, is represented by the formula C-P¹;
each P¹ is, independently, a phosphorothioate internucleoside linkage;
each P² is, independently, a phosphodiester internucleoside linkage;
j is an integer from 1 to 7;
k is an integer from 1 to 7; and
l is an integer from 1 to 7.
   E92. The siRNA molecule of E91, wherein j is from 1 to 6.
   E93. The siRNA molecule of E91, wherein j is from 1 to 5.
   E94. The siRNA molecule of E91, wherein j is from 1 to 4.
   E95. The siRNA molecule of E91, wherein j is from 1 to 3.
   E96. The siRNA molecule of E91, wherein j is from 1 to 2.
   E97. The siRNA molecule of E91, wherein j is 1.
   E98. The siRNA molecule of E91, wherein j is 2.
   E99. The siRNA molecule of E91, wherein j is 3.
   E100. The siRNA molecule of E91, wherein j is 4.
   E101. The siRNA molecule of E91, wherein j is 5.
   E102. The siRNA molecule of E91 or E90, wherein j is 6.
   E103. The siRNA molecule of E91, wherein j is 7.
   E104. The siRNA molecule of any one of E91-E103, wherein k is from 1 to 6.
   E105. The siRNA molecule of E104, wherein k is from 1 to 5.
   E106. The siRNA molecule of E104, wherein k is from 1 to 4.
   E107. The siRNA molecule of E104, wherein k is from 1 to 3.
   E108. The siRNA molecule of E104, wherein k is from 1 to 2.
   E109. The siRNA molecule of E104, wherein k is 1.
   E110. The siRNA molecule of E104, wherein k is 2.
   E111. The siRNA molecule of E104, wherein k is 3.
   E112. The siRNA molecule of E104, wherein k is 4.
   E113. The siRNA molecule of E104, wherein k is 5.
   E114. The siRNA molecule of E104, wherein k is 6.
   E115. The siRNA molecule of E104, wherein k is 7.
   E116. The siRNA molecule of any one of E91-E115, wherein l is from 1 to 6.
   E117. The siRNA molecule of E116, wherein l is from 1 to 5.
   E118. The siRNA molecule of E116, wherein l is from 1 to 4.
   E119. The siRNA molecule of E116, wherein l is from 1 to 3.
   E120. The siRNA molecule of E116, wherein l is from 1 to 2.
   E121. The siRNA molecule of E116, wherein l is 1.
   E122. The siRNA molecule of E116, wherein l is 2.
   E123. The siRNA molecule of E116, wherein l is 3.
   E124. The siRNA molecule of E116, wherein l is 4.
   E125. The siRNA molecule of E116, wherein l is 5.
   E126. The siRNA molecule of E116, wherein l is 6.
   E127. The siRNA molecule of E116, wherein l is 7.
   E128. The siRNA molecule of E91, wherein j is 3, k is 6, and l is 2.
   E129. The siRNA molecule of E91, wherein the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.
   E130. The siRNA molecule of any one of E91-E129, wherein the sense strand includes a structure represented by Formula VII, wherein Formula VII is, in the 5′-to-3′ direction:

H-Bₘ-Iₙ-A′-Bₒ-H-C Formula VII;
wherein A′ is represented by the formula C-P²-D-P²;
each H, independently, is represented by the formula (C-P¹)₂;
each I, independently, is represented by the formula (D-P²);
B, C, D, P¹, and P² are as defined in Formula VI;
m is an integer from 1 to 7;
n is an integer from 1 to 7; and
o is an integer from 1 to 7.
   E131. The siRNA molecule of E130, wherein m is from 1 to 6.
   E132. The siRNA molecule of E130, wherein m is from 1 to 5.
   E133. The siRNA molecule of E130, wherein m is from 1 to 4.
   E134. The siRNA molecule of E130, wherein m is from 1 to 3.
   E135. The siRNA molecule of E130, wherein m is 1 or 2.
   E136. The siRNA molecule of E130, wherein m is 1.
   E137. The siRNA molecule of E130, wherein m is 2.
   E138. The siRNA molecule of E130, wherein m is 3.
   E139. The siRNA molecule of E130, wherein m is 4.
   E140. The siRNA molecule of E130, wherein m is 5.
   E141. The siRNA molecule of E130, wherein m is 6.
   E142. The siRNA molecule of E130, wherein m is 7.
   E143. The siRNA molecule of E130, wherein n is from 1 to 6.
   E144. The siRNA molecule of E130, wherein n is from 1 to 5.
   E145. The siRNA molecule of E130, wherein n is from 1 to 4.
   E146. The siRNA molecule of E130, wherein n is from 1 to 3.
   E147. The siRNA molecule of E130, wherein n is from 1 to 2.
   E148. The siRNA molecule of E130, wherein n is 1.
   E149. The siRNA molecule of E130, wherein n is 2.
   E150. The siRNA molecule of E130, wherein n is 3.
   E151. The siRNA molecule of E130, wherein n is 4.
   E152. The siRNA molecule of E130, wherein n is 5.
   E153. The siRNA molecule of E130, wherein n is 6.
   E154. The siRNA molecule of E130, wherein n is 7.
   E155. The siRNA molecule of any one of E130-E154, wherein o is from 1 to 6.
   E156. The siRNA molecule of E155, wherein o is from 1 to 5.
   E157. The siRNA molecule of E155, wherein o is from 1 to 4.
   E158. The siRNA molecule of E155, wherein o is from 1 to 3.
   E159. The siRNA molecule of E155, wherein o is from 1 to 2.
   E160. The siRNA molecule of E155, wherein o is 1.
   E161. The siRNA molecule of E155, wherein o is 2.
   E162. The siRNA molecule of E155, wherein o is 3.
   E163. The siRNA molecule of E155, wherein o is 4.
   E164. The siRNA molecule of E155, wherein o is 5.
   E165. The siRNA molecule of E155, wherein o is 6.
   E166. The siRNA molecule of E155, wherein o is 7.
   E167. The siRNA molecule of E130, wherein m is 3, n is 3, and o is 3.
   E168. The siRNA molecule of E130, wherein the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.
   E169. The siRNA molecule of any one of E1-E168, wherein P¹ is a phosphorothioate linkage.
   E170. The siRNA molecule of any one of E1-E169, wherein P² is a phosphodiester linkage.
   E171. The siRNA molecule of any one of E1-E170, wherein the antisense strand further includes a 5′ phosphorus stabilizing moiety at the 5′ end of the antisense strand.
   E172. The siRNA molecule of any one of E1-E171, wherein the sense strand further includes a 5′ phosphorus stabilizing moiety at the 5′ end of the sense strand.
   E173. The siRNA molecule of any one of E1-E172, wherein at least 10% of the ribonucleosides are 2′-O-Me ribonucleosides.
   E174. The siRNA molecule of E173, wherein at least 20% of the ribonucleosides are 2′-O-Me ribonucleosides.
   E175. The siRNA molecule of E174, wherein at least 30% of the ribonucleosides are 2′-O-Me ribonucleosides.
   E176. The siRNA molecule of E175, wherein at least 40% of the ribonucleosides are 2′-O-Me ribonucleosides.
   E177. The siRNA molecule of E176, wherein at least 50% of the ribonucleosides are 2′-O-Me ribonucleosides.
   E178. The siRNA molecule of E177, wherein at least 60% of the ribonucleosides are 2′-O-Me ribonucleosides.
   E179. The siRNA molecule of E178, wherein at least 70% of the ribonucleosides are 2′-O-Me ribonucleosides.
   E180. The siRNA molecule of E179, wherein at least 80% of the ribonucleosides are 2′-O-Me ribonucleosides.
   E181. The siRNA molecule of E180, wherein at least 90% of the ribonucleosides are 2′-O-Me ribonucleosides.
   E182. The siRNA molecule of any one of E1-E181, wherein at least 10% of the ribonucleosides are 2′-F ribonucleosides.
   E183. The siRNA molecule of E182, wherein at least 20% of the ribonucleosides are 2′-F ribonucleosides.
   E184. The siRNA molecule of E183, wherein at least 30% of the ribonucleosides are 2′-F ribonucleosides.
   E185. The siRNA molecule of E184, wherein at least 40% of the ribonucleosides are 2′-F ribonucleosides.
   E186. The siRNA molecule of E185, wherein at least 50% of the ribonucleosides are 2′-F ribonucleosides.
   E187. The siRNA molecule of E186, wherein at least 60% of the ribonucleosides are 2′-F ribonucleosides.
   E188. The siRNA molecule of E187, wherein at least 70% of the ribonucleosides are 2′-F ribonucleosides.
   E189. The siRNA molecule of E188, wherein at least 80% of the ribonucleosides are 2′-F ribonucleosides.
   E190. The siRNA molecule of E189, wherein at least 90% of the ribonucleosides are 2′-F ribonucleosides.
   E191. The siRNA molecule of any one of E1-E190, wherein the antisense strand has 12 2′-O-Me ribonucleosides.
   E192. The siRNA molecule of any one of E1-E191, wherein the antisense strand has nine 2′-F ribonucleosides.
   E193. The siRNA molecule of E191 or E192, wherein the sense strand has 11 2′-O-Me ribonucleosides.
   E194. The siRNA molecule of E191 or E192, wherein the sense strand has 10 2′-O-Me ribonucleosides.
   E195. The siRNA molecule of any one of E191-E194, wherein the sense strand has five 2′-F ribonucleosides.
   E196. The siRNA molecule of any one of E191-E194, wherein the sense strand has six 2′-F ribonucleosides.
   E197. The siRNA molecule of any one of E1-E190, wherein the antisense strand has 17 2′-O-Me ribonucleosides.
   E198. The siRNA molecule of any one of E1-E190 or E197, wherein the antisense strand has five 2′-F ribonucleosides.
   E199. The siRNA molecule of any one of E197 and E198, wherein the sense strand has 12 2′-O-Me ribonucleosides.
   E200. The siRNA molecule of any one of E197-E199, wherein the sense strand has four 2′-F ribonucleosides.
   E201. The siRNA molecule of any one of E1-E200, wherein at least 10% of the internucleoside linkages are phosphodiester linkages or phosphorothioate linkages.
   E202. The siRNA molecule of E201, wherein at least 20% of the internucleoside linkages are phosphodiester linkages or phosphorothioate linkages.
   E203. The siRNA molecule of E202, wherein at least 30% of the internucleoside linkages are phosphodiester linkages or phosphorothioate linkages.
   E204. The siRNA molecule of E203, wherein at least 40% of the internucleoside linkages are phosphodiester linkages or phosphorothioate linkages.
   E205. The siRNA molecule of E204, wherein at least 50% of the internucleoside linkages are phosphodiester linkages or phosphorothioate linkages.
   E206. The siRNA molecule of E205, wherein at least 60% of the internucleoside linkages are phosphodiester linkages or phosphorothioate linkages.
   E207. The siRNA molecule of E206, wherein at least 70% of the internucleoside linkages are phosphodiester linkages or phosphorothioate linkages.
   E208. The siRNA molecule of E207, wherein at least 80% of the internucleoside linkages are phosphodiester linkages or phosphorothioate linkages.
   E209. The siRNA molecule of E208, wherein at least 90% of the internucleoside linkages are phosphodiester linkages or phosphorothioate linkages.
   E210. The siRNA molecule of any one of E1-E209, wherein the antisense strand has seven
   phosphorothioate linkages.
   E211. The siRNA molecule of any one of E1-E210, wherein the antisense strand has 13 phosphodiester linkages.
   E212. The siRNA molecule of E210 or E211, wherein the sense strand has 4 phosphorothioate linkages.
   E213. The siRNA molecule of any one of E210-E212, wherein the sense strand has 11 phosphodiester linkages.
   E214. The siRNA molecule of any one of E210-E212, wherein the sense strand has 13 phosphodiester linkages.
   E215. The siRNA molecule of any one of E171-E214, wherein the 5′ phosphorus stabilizing moiety is represented by any one of Formulas VIII-XV:

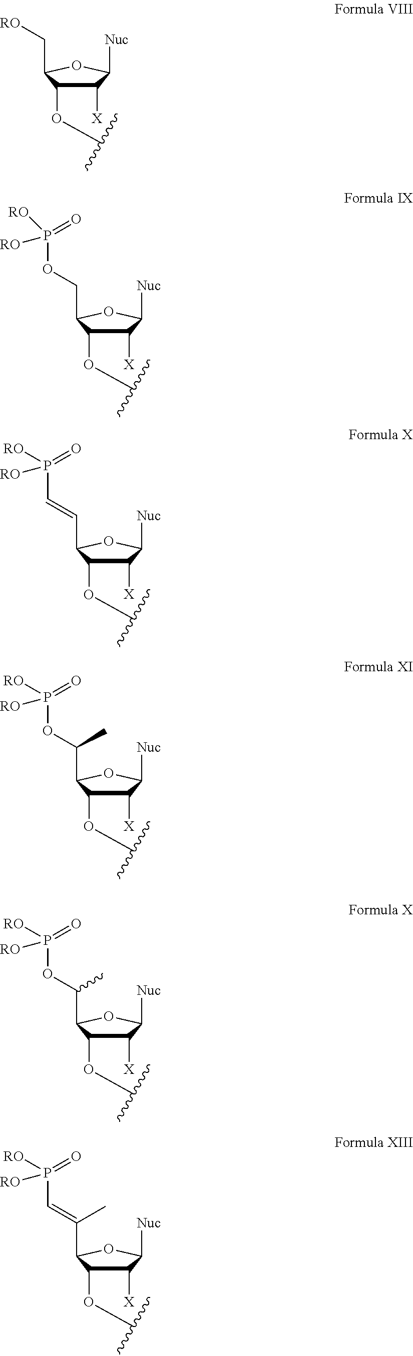

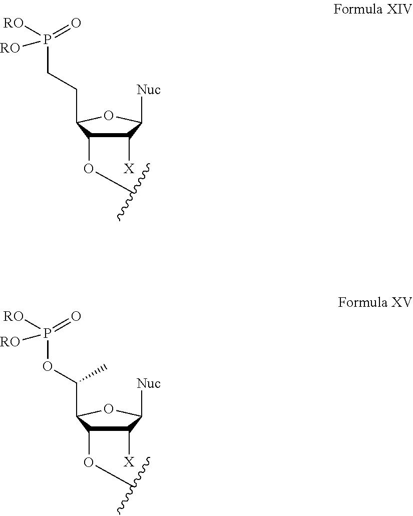

wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.
   E216. The siRNA molecule of E215, wherein the nucleobase is an adenine, uracil, guanine, thymine, or cytosine.
   E217. The siRNA molecule of E215 or E216, wherein the 5′ phosphorus stabilizing moiety is (E)-vinylphosphonate represented by Formula X.
   E218. The siRNA molecule of any one of E1-E217, wherein the siRNA molecule further includes a hydrophobic moiety at the 5′ or the 3′ end of the siRNA molecule.
   E219. The siRNA molecule of E218, wherein the hydrophobic moiety is at the 5′ end of the siRNA molecule. E220. The siRNA molecule of E218 or E219, wherein the hydrophobic moiety is at the 3′ end of the siRNA molecule.
   E221. The siRNA molecule of any one of E218-E220, wherein the hydrophobic moiety is selected from a group consisting of cholesterol, vitamin D, or tocopherol.
   E222. The siRNA molecule of E221, wherein the hydrophobic moiety is cholesterol.
   E223. The siRNA molecule of E221, wherein the hydrophobic moiety is vitamin D.
   E224. The siRNA molecule of E221, wherein the hydrophobic moiety is tocopherol.
   E225. The siRNA molecule of any one of E1-E224, wherein the length of the antisense strand is between 10 and 30 nucleotides (e.g., 11, 12, 13, 14, 15, 16, 17, 18, 19, 20, 21, 22, 23, 24, 25, 26, 27, 28, or 29 nucleotides).
   E226. The siRNA molecule of E225, wherein the length of the antisense strand is between 15 and 30 nucleotides (e.g., 16, 17, 18, 19, 20, 21, 22, 23, 24, 25, 26, 27, 28, or 29 nucleotides).
   E227. The siRNA molecule of E226, wherein the length of the antisense strand is between 18 and 23 nucleotides (e.g., 19, 20, 21, or 22 nucleotides).
   E228. The siRNA molecule of E227, wherein the length of the antisense strand is 20 nucleotides.
   E229. The siRNA molecule of E227, wherein the length of the antisense strand is 21 nucleotides.
   E230. The siRNA molecule of E227, wherein the length of the antisense strand is 22 nucleotides.
   E231. The siRNA molecule of E227, wherein the length of the antisense strand is 23 nucleotides.
   E232. The siRNA molecule of E226, wherein the length of the antisense strand is 24 nucleotides.
   E233. The siRNA molecule of E226, wherein the length of the antisense strand is 25 nucleotides.
   E234. The siRNA molecule of E226, wherein the length of the antisense strand is 26 nucleotides.
   E235. The siRNA molecule of E226, wherein the length of the antisense strand is 27 nucleotides.
   E236. The siRNA molecule of E226, wherein the length of the antisense strand is 28 nucleotides.
   E237. The siRNA molecule of E226, wherein the length of the antisense strand is 29 nucleotides.
   E238. The siRNA molecule of E226, wherein the length of the antisense strand is 30 nucleotides.
   E239. The siRNA molecule of any one of E1-E238, wherein the length of the sense strand is between 12 and 30 nucleotides.
   E240. The siRNA molecule of E239, wherein the length of the sense strand is between 14 and 24 nucleotides (e.g., 15, 16, 17, 18, 19, 20, 21, 22, or 23 nucleotides).
   E241. The siRNA molecule of E240, wherein the length of the sense strand is 15 nucleotides.
   E242. The siRNA molecule of E240, wherein the length of the sense strand is 16 nucleotides.
   E243. The siRNA molecule of E240, wherein the length of the sense strand is 17 nucleotides.
   E244. The siRNA molecule of E240, wherein the length of the sense strand is 18 nucleotides.
   E245. The siRNA molecule of E240, wherein the length of the sense strand is 19 nucleotides.
   E246. The siRNA molecule of E240, wherein the length of the sense strand is 20 nucleotides.
   E247. The siRNA molecule of E240, wherein the length of the sense strand is 21 nucleotides.
   E248. The siRNA molecule of E240, wherein the length of the sense strand is 22 nucleotides.
   E249. The siRNA molecule of E240, wherein the length of the sense strand is 23 nucleotides.
   E250. The siRNA molecule of E240, wherein the length of the sense strand is 24 nucleotides.
   E251. The siRNA molecule of E239, wherein the length of the sense strand is 25 nucleotides.
   E252. The siRNA molecule of E239, wherein the length of the sense strand is 26 nucleotides.
   E253. The siRNA molecule of E239, wherein the length of the sense strand is 27 nucleotides.
   E254. The siRNA molecule of E239, wherein the length of the sense strand is 28 nucleotides.
   E255. The siRNA molecule of E239, wherein the length of the sense strand is 29 nucleotides.
   E256. The siRNA molecule of E239, wherein the length of the sense strand is 30 nucleotides.
   E257. The siRNA molecule of any one of E1-E256, wherein the siRNA molecule is a branched siRNA molecule.
   E258. The siRNA molecule of E257, wherein the branched siRNA molecule is di-branched, tri-branched, or tetra-branched.
   E259. The siRNA molecule of E258, wherein the siRNA molecule is di-branched.
   E260. The siRNA molecule of E258, wherein the siRNA molecule is tri-branched.
   E261. The siRNA molecule of E258, wherein the siRNA molecule is tetra-branched.
   E262. The siRNA molecule of E258 or E259, wherein the di-branched siRNA molecule is represented by any one of Formulas XVI-XVIII:

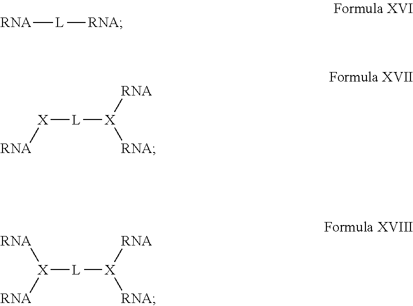

wherein each RNA is, independently, an siRNA molecule, L is a linker, and each X, independently, represents a branch point moiety.
   E263. The siRNA molecule of E262, wherein the di-branched siRNA molecule is represented by Formula XVI.
   E264. The siRNA molecule of E262, wherein the di-branched siRNA molecule is represented by Formula XVII.
   E265. The siRNA molecule of E262, wherein the di-branched siRNA molecule is represented by Formula XVIII.
   E266. The siRNA molecule of E258 or E260, wherein the tri-branched siRNA molecule is represented by any one of Formulas XIX-XXII:

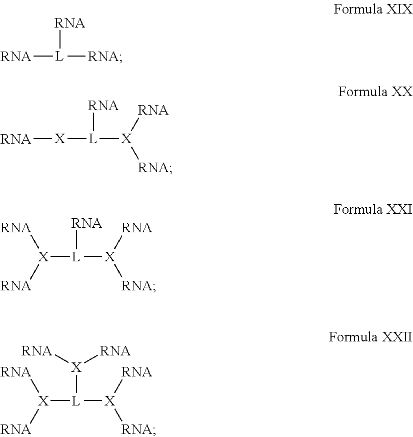

wherein each RNA is, independently, an siRNA molecule, L is a linker, and each X, independently, represents a branch point moiety.
   E267. The siRNA molecule of E266, wherein the tri-branched siRNA molecule is represented by Formula XIX.
   E268. The siRNA molecule of E266, wherein the tri-branched siRNA molecule is represented by Formula XX.
   E269. The siRNA molecule of E266, wherein the tri-branched siRNA molecule is represented by Formula XXI.
   E270. The siRNA molecule of E266, wherein the tri-branched siRNA molecule is represented by Formula XXII.
   E271. The siRNA molecule of E258 or E261, wherein the tetra-branched siRNA molecule is represented by any one of Formulas XXIII-XXVII:

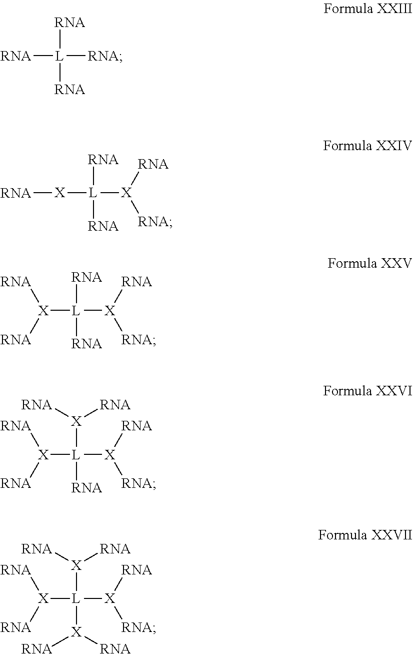

wherein each RNA is, independently, an siRNA molecule, L is a linker, and each X, independently, represents a branch point moiety.
   E272. The siRNA molecule of E271, wherein the tetra-branched siRNA molecule is represented by Formula XXIII.
   E273. The siRNA molecule of E271, wherein the tetra-branched siRNA molecule is represented by Formula XXIV.
   E274. The siRNA molecule of E271, wherein the tetra-branched siRNA molecule is represented by Formula XXV.
   E275. The siRNA molecule of E271, wherein the tetra-branched siRNA molecule is represented by Formula XXVI.
   E276. The siRNA molecule of E271, wherein the tetra-branched siRNA molecule is represented by Formula XXVII.
   E277. The siRNA molecule of any one of E262-E276, wherein the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.
   E278. The siRNA molecule of E277, wherein the linker is an ethylene glycol oligomer.
   E279. The siRNA molecule of E278, wherein the ethylene glycol oligomer is a PEG.
   E280. The siRNA molecule of E279, wherein the PEG a TrEG.
   E281. The siRNA molecule of E279, wherein the PEG is a TEG.
   E282. The siRNA molecule of E277, wherein the linker is an alkyl oligomer.
   E283. The siRNA molecule of E277, wherein the linker is a carbohydrate oligomer.
   E284. The siRNA molecule of E277, wherein the linker is a block copolymer.
   E285. The siRNA molecule of E277, wherein the linker is a peptide oligomer.
   E286. The siRNA molecule of E277, wherein the linker is an RNA oligomer.
   E287. The siRNA molecule of E277, wherein the linker is a DNA oligomer.
   E288. The siRNA molecule of any one of E277-E287, wherein the oligomer or copolymer contains 2 to contiguous subunits.
   E289. The siRNA molecule of E288, wherein oligomer or copolymer contains 4 to 18 contiguous subunits.
   E290. The siRNA molecule of E289, wherein oligomer or copolymer contains 6 to 16 contiguous subunits.
   E291. The siRNA molecule of E230, wherein oligomer or copolymer contains 8 to 14 contiguous subunits.
   E292. The siRNA molecule of E231, wherein oligomer or copolymer contains 10 to 12 contiguous subunits.
   E293. The siRNA molecule of E277, wherein the linker attaches one or more (e.g., 1, 2, or more) siRNA molecules by way of a covalent bond-forming moiety.
   E294. The siRNA molecule of E293, wherein the covalent bond-forming moiety is selected from the group consisting of an alkyl, ester, amide, carbamate, phosphonate, phosphate, phosphorothioate, phosphoroamidate, triazole, urea, and formacetal.
   E295. The siRNA molecule of E277, wherein the linker includes a structure of Formula L1, wherein Formula L1 is:

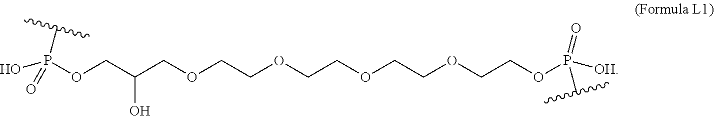 [SMILES: *P(=O)(O)OCCOCCOCCOCCOCC(O)COP(*)(=O)O]

E296. The siRNA molecule of E277, wherein the linker includes a structure of Formula L2, wherein Formula L2 is:

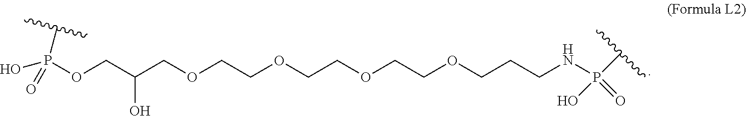 [SMILES: *P(=O)(O)NCCCOCCOCCOCCOCC(O)COP(*)(=O)O]

E297. The siRNA molecule of E277, wherein the linker includes a structure of Formula L3, wherein Formula L3 is:

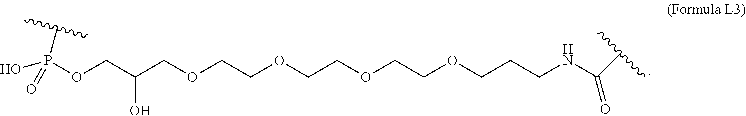 [SMILES: *C(=O)NCCCOCCOCCOCCOCC(O)COP(*)(=O)O]

E298. The siRNA molecule of E277, wherein the linker includes a structure of Formula L4, wherein Formula L4 is:

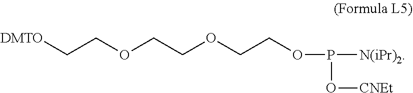

E299. The siRNA molecule of E277, wherein the linker includes a structure of Formula L5, wherein Formula L5 is:

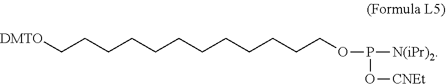

E300. The siRNA molecule of E277, wherein the linker includes a structure of Formula L6, wherein Formula L6 is:

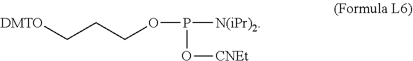

E301. The siRNA molecule of E277, wherein the linker includes a structure of Formula L7, wherein Formula L7 is:

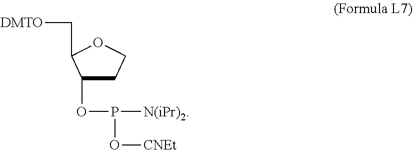

E302. The siRNA molecule of E277, wherein the linker includes a structure of Formula L8, wherein Formula L8 is:

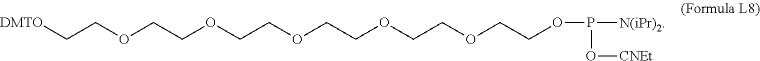

E303. The siRNA molecule of E277, wherein the linker includes a structure of Formula L9, wherein Formula L9 is:

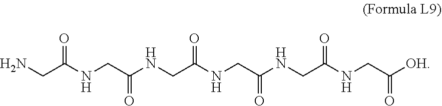 [SMILES: NCC(=O)NCC(=O)NCC(=O)NCC(=O)NCC(=O)NCC(=O)O]

E304. The siRNA molecule of any one of E1-E303, wherein the antisense strand has complementarity sufficient to hybridize a portion of a gene selected from ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.
E305. The siRNA molecule of E304, wherein the antisense strand has complementarity sufficient to hybridize a portion of a gene selected from APOE, BIN1, C1QA, C3, C9ORF72, CCL5, CD33, CLU/APOJ, CR1, CXCL10, CXCL13, IFIT1, IFIT3, IFITM3, IFNAR1, IFNAR2, IL10RA, IL1A, IL1B, IL1RAP, INPP5D, ITGAM, MEF2C, MMP12, NLRP3, NOS2, PILRA, PLCG2, PTK2B, SLC24A4, TBK1, and TNF.
E306. The siRNA molecule of E304, wherein the antisense strand has complementarity sufficient to hybridize a portion of a gene selected from HTT, MAPT, SNCA, C9ORF72, APOE, SCN9A, KCNT1, PRNP, and MSH3.
E307. The siRNA molecule of E306, wherein the antisense strand has complementarity sufficient to hybridize a portion of an HTT gene.
E308. The siRNA molecule of E306, wherein the antisense strand has complementarity sufficient to hybridize a portion of an MAPT gene.
E309. The siRNA molecule of E306, wherein the antisense strand has complementarity sufficient to hybridize a portion of an SNCA gene.
E310. The siRNA molecule of E306, wherein the antisense strand has complementarity sufficient to hybridize a portion of an C9ORF72 gene.
E311. The siRNA molecule of E306, wherein the antisense strand has complementarity sufficient to hybridize a portion of an APOE gene.
E312. The siRNA molecule of E306, wherein the antisense strand has complementarity sufficient to hybridize a portion of an SCN9A gene.
E313. The siRNA molecule of E306, wherein the antisense strand has complementarity sufficient to hybridize a portion of a KCNT1 gene.
E314. The siRNA molecule of E306, wherein the antisense strand has complementarity sufficient to hybridize a portion of a PRNP gene.
E315. The siRNA molecule of E306, wherein the antisense strand has complementarity sufficient to hybridize a portion of an MSH3 gene.
E316. A pharmaceutical composition including the siRNA molecule of any one of E1-E315, and a pharmaceutically acceptable excipient, carrier, or diluent.
E317. A method of delivering an siRNA molecule to the central nervous system (CNS) of a subject, the method including administering the siRNA molecule of any one of E1-E315 or the pharmaceutical composition of E302 to the CNS of the subject.
E318. The method of E317, wherein the siRNA molecule or the pharmaceutical composition is administered to the subject by way of intrastriatal, intracerebroventricular, or intrathecal injection.
E319. The method of E317 or E318, wherein the delivering the siRNA molecule or the pharmaceutical composition to the CNS of the subject results in gene silencing of a target gene in the subject.
E320. The method of E319, wherein the target gene is an overactive disease driver gene.
E321. The method of E319, wherein the target gene is a negative regulator of a gene with reduced expression that is associated with a disease state in the subject.
E322. The method of E319, wherein the target gene is a positive regulator of a gene with increased expression that is associated with a disease state in the subject.
E323. The method of E319, wherein the target gene is a splice isoform of the target gene, wherein the splice isoform reduces expression of the target gene.
E324. The method of any one of E319-E323, wherein the gene silencing treats a disease state in the subject.
E325. The method of any one of E317-E324, wherein the siRNA molecule or the pharmaceutical composition is administered to the subject by way of intrastriatal injection.
E326. The method of E317-E324, wherein the siRNA molecule or the pharmaceutical composition is administered to the subject by way of intracerebroventricular injection.
E327. The method of E317-E324, wherein the siRNA molecule or the pharmaceutical composition is administered to the subject by way of intrathecal injection.
E328. The method of any one of E317-E327, wherein the subject is a human.
E329. A kit including the siRNA molecule of any one of E1-E315, or the pharmaceutical composition of
E316, and a package insert, wherein the package insert instructs a user of the kit to perform the method of any one of E317-E328.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A small interfering RNA (siRNA) molecule comprising an antisense strand and a sense strand having complementarity to the antisense strand, wherein the antisense strand comprises a structure represented by Formula I, wherein Formula I is, in the 5′-to-3′ direction:
A-B-(A′)ⱼ-C-P²-D-P¹-(C′-P¹)ₖ-C′ Formula I;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′-O-methyl (2′-O-Me) ribonucleoside;
each C′, independently, is a 2′-O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.

2. The siRNA molecule of claim 1, wherein the antisense strand comprises a structure represented by Formula A1, wherein Formula A1 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

3. A small interfering RNA (siRNA) molecule comprising an antisense strand and a sense strand having complementarity to the antisense strand, wherein the antisense strand comprises a structure represented by Formula II, wherein Formula II is, in the 5′-to-3′ direction:
A-B-(A′)ⱼ-C-P²-D-P¹-(C-P¹)ₖ-C′ Formula II;
wherein A is represented by the formula C-P¹-D-P¹;
each A′, independently, is represented by the formula C-P²-D-P²;
B is represented by the formula C-P²-D-P²-D-P²-D-P²;
each C, independently, is a 2′-O-methyl (2′-O-Me) ribonucleoside;
each C′, independently, is a 2′-O-Me ribonucleoside or a 2′-fluoro (2′-F) ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.

4. The siRNA molecule of claim 3, wherein the antisense strand comprises a structure represented by Formula A2, wherein Formula A2 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

5. The siRNA molecule of any one of claims 1-4, wherein the sense strand comprises a structure represented by Formula III, wherein Formula III is, in the 5′-to-3′ direction:
E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7.

6. The siRNA molecule of any one of claims 1-5, wherein j is 4 and k is 4.

7. The siRNA molecule of claim 5 or claim 6, wherein m is 4.

8. The siRNA molecule of claim 5, wherein the sense strand comprises a structure represented by Formula S1, wherein Formula S1 is, in the 5′-to-3′ direction:
A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

9. The siRNA molecule of claim 5, wherein the sense strand comprises a structure represented by Formula S2, wherein Formula S2 is, in the 5′-to-3′ direction:
A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

10. The siRNA molecule of claim 5, wherein the sense strand comprises a structure represented by Formula S3, wherein Formula S3 is, in the 5′-to-3′ direction:
A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

11. The siRNA molecule of claim 5, wherein the sense strand comprises a structure represented by Formula S4, wherein Formula S4 is, in the 5′-to-3′ direction:
A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

12. An siRNA molecule comprising an antisense strand and a sense strand having complementarity to the antisense strand, wherein the antisense strand comprises a structure represented by Formula IV, wherein Formula IV is, in the 5′-to-3′ direction:
A-(A′)ⱼ-C-P²-B-(C-P¹)ₖ-C′ Formula IV;
wherein A is represented by the formula C-P¹-D-P¹;
each A′, independently, is represented by the formula C-P²-D-P²;
B is represented by the formula D-P¹-C-P¹-D-P^{1;}
each C, independently, is a 2′-O-Me ribonucleoside;
each C′, independently, is a 2′-O-Me ribonucleoside or a 2′-F ribonucleoside;
each D, independently, is a 2′-F ribonucleoside;
each P¹ is, independently, a phosphorothioate internucleoside linkage;
each P² is, independently, a phosphodiester internucleoside linkage;
j is an integer from 1 to 7; and
k is an integer from 1 to 7.

13. The siRNA molecule of claim 12, wherein the antisense strand comprises a structure represented by Formula A3, wherein Formula A3 is, in the 5′-to-3′ direction:
A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

14. The siRNA molecule of claim 12 or claim 13, wherein the sense strand comprises a structure represented by Formula V, wherein Formula V is, in the 5′-to-3′ direction:
E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7.

15. The siRNA molecule of claim 14, wherein j is 6 and k is 2.

16. The siRNA molecule of claim 14 or claim 15, wherein m is 5.

17. The siRNA molecule of claim 14, wherein the sense strand comprises a structure represented by Formula S5, wherein Formula S5 is, in the 5′-to-3′ direction:
A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

18. The siRNA molecule of claim 14, wherein the sense strand comprises a structure represented by Formula S6, wherein Formula S6 is, in the 5′-to-3′ direction:
A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

19. The siRNA molecule of claim 14, wherein the sense strand comprises a structure represented by Formula S7, wherein Formula S7 is, in the 5′-to-3′ direction:
A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

20. The siRNA molecule of claim 14, wherein the sense strand comprises a structure represented by Formula S8, wherein Formula S8 is, in the 5′-to-3′ direction:
A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

21. An siRNA molecule comprising an antisense strand and a sense strand having complementarity to the antisense strand, wherein the antisense strand comprises a structure represented by Formula VI, wherein Formula VI is, in the 5′-to-3′ direction:
A-Bⱼ-E-Bₖ-E-F-Gₗ-D-P¹-C′ Formula VI;
wherein A is represented by the formula C-P¹-D-P¹;
each B, independently, is represented by the formula C-P²;
each C, independently, is a 2′-O-Me ribonucleoside;
each C′, independently, is a 2′-O-Me ribonucleoside or a 2′-F ribonucleoside;
each D, independently, is a 2′-F ribonucleoside;
each E, independently, is represented by the formula D-P²-C-P²;
F is represented by the formula D-P¹-C-P¹;
each G, independently, is represented by the formula C-P¹;
each P¹ is, independently, a phosphorothioate internucleoside linkage;
each P² is, independently, a phosphodiester internucleoside linkage;
j is an integer from 1 to 7;
k is an integer from 1 to 7; and
l is an integer from 1 to 7.

22. The siRNA molecule of claim 21, wherein the antisense strand comprises a structure represented by Formula A4, wherein Formula A4 is, in the 5′-to-3′ direction:
A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

23. The siRNA molecule of claim 21 or claim 22, wherein the sense strand comprises is represented by Formula VII, wherein Formula VII is, in the 5′-to-3′ direction:
H-Bₘ-Iₙ-A′-Bₒ-H-C Formula VII;
wherein A′ is represented by the formula C-P²-D-P²;
each H, independently, is represented by the formula (C-P¹)₂;
each I, independently, is represented by the formula (D-P²);
B, C, D, P¹ and P² are as defined in Formula VI;
m is an integer from 1 to 7;
n is an integer from 1 to 7; and
o is an integer from 1 to 7.

24. The siRNA molecule of claim 23, wherein j is 3, k is 6, and l is 2.

25. The siRNA molecule of claim 23 or claim 24, wherein m is 3, n is 3, and o is 3.

26. The siRNA molecule of claim 23, wherein the sense strand comprises a structure represented by Formula S9, wherein Formula S9 is, in the 5′-to-3′ direction:
A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

27. The siRNA molecule of any one of claims 1-26, wherein the antisense strand further comprises a 5′ phosphorus stabilizing moiety at the 5′ end of the antisense strand.

28. The siRNA molecule of any one of claims 1-27, wherein the sense strand further comprises a 5′ phosphorus stabilizing moiety at the 5′ end of the sense strand.

29. The siRNA molecule of claim 27 or claim 28, wherein the 5′ phosphorus stabilizing moiety is represented by any one of Formulas VIII-XV: 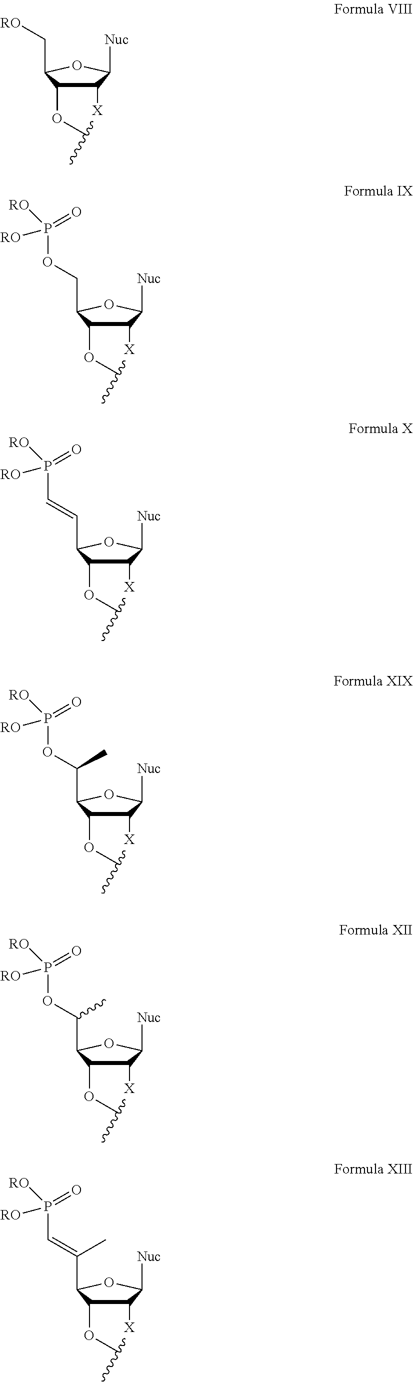 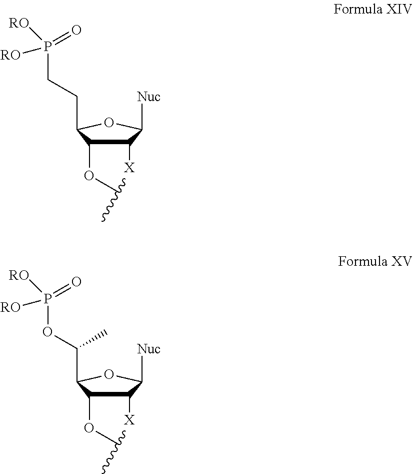
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

30. The siRNA molecule of claim 29, wherein the nucleobase is an adenine, uracil, guanine, thymine, or cytosine.

31. The siRNA molecule of any one of claims 27-30, wherein the 5′ phosphorus stabilizing moiety is (E)-vinylphosphonate represented by Formula X.

32. The siRNA molecule of any one of claims 1-31, wherein the siRNA molecule further comprises a hydrophobic moiety at the 5′ or the 3′ end of the siRNA molecule.

33. The siRNA molecule of claim 32, wherein the hydrophobic moiety is selected from a group consisting of cholesterol, vitamin D, or tocopherol.

34. The siRNA molecule of any one of claims 1-33, wherein the length of the antisense strand is between 10 and 30 nucleotides.

35. The siRNA molecule of claim 34, wherein the length of the antisense strand is between 15 and 25 nucleotides.

36. The siRNA molecule of claim 35, wherein the length of the antisense strand is 20 nucleotides.

37. The siRNA molecule of claim 35, wherein the length of the antisense strand is 21 nucleotides.

38. The siRNA molecule of claim 35, wherein the length of the antisense strand is 22 nucleotides.

39. The siRNA molecule of claim 35, wherein the length of the antisense strand is 23 nucleotides.

40. The siRNA molecule of claim 35, wherein the length of the antisense strand is 24 nucleotides.

41. The siRNA molecule of claim 35, wherein the length of the antisense strand is 25 nucleotides.

42. The siRNA molecule of claim 34, wherein the length of the antisense strand is 26 nucleotides.

43. The siRNA molecule of claim 34, wherein the length of the antisense strand is 27 nucleotides.

44. The siRNA molecule of claim 34, wherein the length of the antisense strand is 28 nucleotides.

45. The siRNA molecule of claim 34, wherein the length of the antisense strand is 29 nucleotides.

46. The siRNA molecule of claim 34, wherein the length of the antisense strand is 30 nucleotides.

47. The siRNA molecule of any one of claims 1-46, wherein the length of the sense strand is between 12 and 30 nucleotides.

48. The siRNA molecule of claim 47, wherein the length of the sense strand is 15 nucleotides.

49. The siRNA molecule of claim 47, wherein the length of the sense strand is 16 nucleotides.

50. The siRNA molecule of claim 47, wherein the length of the sense strand is 17 nucleotides.

51. The siRNA molecule of claim 47, wherein the length of the sense strand is 18 nucleotides.

52. The siRNA molecule of claim 47, wherein the length of the sense strand is 19 nucleotides.

53. The siRNA molecule of claim 47, wherein the length of the sense strand is 20 nucleotides.

54. The siRNA molecule of claim 47, wherein the length of the sense strand is 21 nucleotides.

55. The siRNA molecule of claim 47, wherein the length of the sense strand is 22 nucleotides.

56. The siRNA molecule of claim 47, wherein the length of the sense strand is 23 nucleotides.

57. The siRNA molecule of claim 47, wherein the length of the sense strand is 24 nucleotides.

58. The siRNA molecule of claim 47, wherein the length of the sense strand is 25 nucleotides.

59. The siRNA molecule of claim 47, wherein the length of the sense strand is 26 nucleotides.

60. The siRNA molecule of claim 47, wherein the length of the sense strand is 27 nucleotides.

61. The siRNA molecule of claim 47, wherein the length of the sense strand is 28 nucleotides.

62. The siRNA molecule of claim 47, wherein the length of the sense strand is 29 nucleotides.

63. The siRNA molecule of claim 47, wherein the length of the sense strand is 30 nucleotides.

64. The siRNA molecule of any one of claims 1-63, wherein the siRNA molecule is a branched siRNA molecule.

65. The siRNA molecule of claim 64, wherein the branched siRNA molecule is di-branched, tri-branched, or tetra-branched.

66. The siRNA molecule of claim 65, wherein the di-branched siRNA molecule is represented by any one of Formulas XVI-XVIII: 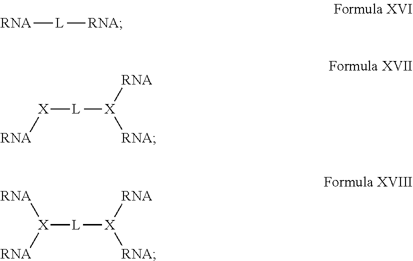
wherein each RNA is, independently, an siRNA molecule, L is a linker, and each X, independently, represents a branch point moiety.

67. The siRNA molecule of claim 65, wherein the tri-branched siRNA molecule is represented by any one of Formulas XIX-XXII: 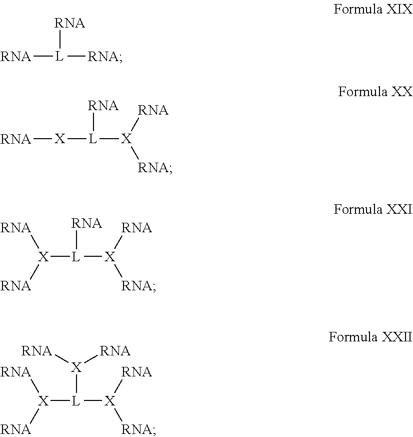
wherein each RNA is, independently, an siRNA molecule, L is a linker, and each X, independently, represents a branch point moiety.

68. The siRNA molecule of claim 65, wherein the tetra-branched siRNA molecule is represented by any one of Formulas XXIII-XXVII: 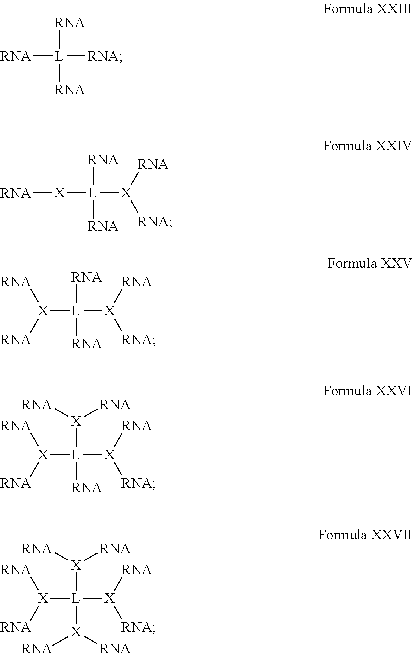
wherein each RNA is, independently, an siRNA molecule, L is a linker, and each X, independently, represents a branch point moiety.

69. The siRNA molecule of any one of claims 66-68, wherein the linker is selected from a group consisting of one or more contiguous subunits of an ethylene glycol, alkyl, carbohydrate, block copolymer, peptide, RNA, and DNA.

70. The siRNA molecule of claim 69, wherein the one or more contiguous subunits is 2 to 20 contiguous subunits.

71. The siRNA molecule of any one of claims 1-70, wherein the antisense strand has complementarity sufficient to hybridize a portion of a gene selected from the group consisting of ABCA7, ABI3, ADAM10, APOC1, APOE, AXL, BIN1, C1QA, C3, C9ORF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

72. The siRNA molecule of claim 71, wherein the gene is selected from the group consisting of HTT, MAPT, SNCA, C9ORF72, APOE, SCN9A, KCNT1, PRNP, and MSH3.

73. The siRNA molecule of claim 72, wherein the gene is HTT.

74. A pharmaceutical composition comprising the siRNA molecule of any one of claims 1-73, and a pharmaceutically acceptable excipient, carrier, or diluent.

75. A method of delivering an siRNA molecule to the central nervous system (CNS) of a subject, the method comprising administering the siRNA molecule of any one of claims 1-73 or the pharmaceutical composition of claim 74 to the CNS of the subject.

76. The method of claim 75, wherein the siRNA molecule or the pharmaceutical composition is administered to the subject by way of intrastriatal, intracerebroventricular, or intrathecal injection.

77. The method of claim 75 or claim 76, wherein the delivering of the siRNA molecule or the pharmaceutical composition to the CNS of the subject results in gene silencing of a target gene in the subject.

78. The method of claim 77, wherein the target gene is an overactive disease driver gene.

79. The method of claim 77, wherein the target gene is a negative regulator of a gene with reduced expression that is associated with a disease state in the subject.

80. The method of claim 77, wherein the target gene is a positive regulator of a gene with increased expression that is associated with a disease state in a subject.

81. The method of claim 77, wherein the target gene is a splice isoform of the target gene, wherein the splice isoform reduces expression of the target gene.

82. The method of any one of claims 77-81, wherein the gene silencing treats a disease state in the subject.

83. The method of any one of claims 75-82, wherein the subject is a human.

84. A kit comprising the siRNA molecule of any one of claims 1-73, or the pharmaceutical composition of claim 74, and a package insert, wherein the package insert instructs a user of the kit to perform the method of any one of claims 75-82.
